SEQUENCE LISTING (85 sequences)

SEQ ID NO: 1 - DNA, 1020 bp, Bacillus subtilis
  Inventor Chiba, Yasunori Inventor Amano, Kou Inventor Jigami, Yoshifumi Inventor Kobayashi, Kazuo
atggcaatacttgttactggcggtgccggttacattggcagccacacatgtgttgaactattgaacagcggctacgagattgttgttcttgataatctgtccaacagttcagctgaagcgctgaaccgtgtcaaggagattacaggaaaagatttaacgttctacgaagcggatttattggaccgggaagcggtagattccgtttttgctgaaaatgaaatcgaagctgtgattcattttgcagggttaaaagcagtcggcgaatctgtggcgattcccctcaaatattatcataacaatttgacaggaacgtttattttatgcgaggccatggagaaatacggcgtcaagaaaatcgtattcagttcatctgcgacagtatacggcgttccggaaacatcgccgattacggaagactttccattaggcgcgacaaatccttatgggcagacgaagctcatgcttgaacaaatattgcgtgatttgcatacagccgacaatgagtggagcgttgcgctgcttcgttactttaacccgttcggcgcgcatccaagcggacggatcggtgaagacccgaacggaatcccaaataaccttatgccgtatgtggcacaggtagcagtcgggaagctcgagcaattaagcgtattcggaaatgactatccgacaaaagacgggacaggcgtacgcgattatattcacgtcgttgatctcgcagaaggccacgtcaaggcgctggaaaaagtattgaactctacaggagccgatgcatacaaccttggaacaggcacaggctacagcgtgctggaaatggtcaaagcctttgaaaaagtgtcagggaaagaggttccataccgttttgcggaccgccgtccgggagacatcgccacatgctttgcagatcctgcgaaagccaagcgagaactaggctgggaagcgaaacgcggccttgaggaaatgtgtgctgattcctggagatggcagtcttctaatgtgaatgggtataagagtgcggaataa

SEQ ID NO: 2 - protein, 339 aa, Bacillus subtilis
MAILVTGGAGYIGSHTCVELLNSGYEIVVLDNLSNSSAEALNRVKEITGKDLTFYEADLLDREAVDSVFAENEIEAVIHFAGLKAVGESVAIPLKYYHNNLTGTFILCEAMEKYGVKKIVFSSSATVYGVPETSPITEDFPLGATNPYGQTKLMLEQILRDLHTADNEWSVALLRYFNPFGAHPSGRIGEDPNGIPNNLMPYVAQVAVGKLEQLSVFGNDYPTKDGTGVRDYIHVVDLAEGHVKALEKVLNSTGADAYNLGTGTGYSVLEMVKAFEKVSGKEVPYRFADRRPGDIATCFADPAKAKRELGWEAKRGLEEMCADSWRWQSSNVNGYKSAE

SEQ ID NO: 3 - DNA, 1191 bp, Homo sapiens
atggcagcggttggggctggtggttccaccgcggcgcccgggccaggggcggtttccgcgggtgcattggagccggggaccgccagtgcggctcacaggcgcctgaagtacatatccctagctgtgctggtggtccagaatgcctccctcatcctcagcatccgctacgcccgcacgttgccaggggaccgcttctttgccaccactgctgtggtcatggcggaagtgctcaaaggtctcacctgcctgctgctgctcttcgcacagaagaggggtaacgtgaagcacctggttctcttcctccatgaggctgtcctggtgcagtatgtggacacgctcaagctcgcagtgccctctctcatctacaccttgcagaataacctccagtatgttgccatctctaacctaccagctgccactttccaggtgacataccagctgaagatcctgaccacagcgctgttctccgtgctcatgctgaatcgcagcctttcccggctgcagtgggcctccctgctgctcctcttcactggcgtcgccattgtccaggcacagcaagccggtgggggaggcccacggccactggatcagaaccctggggcaggcctggcagccgtcgtggcctcctgtctctcctccggcttcgcaggtgtctactttgagaagatcctcaaaggcagctcaggctccgtgtggctgcgcaacctgcaactgggcctcttcggcacagcactgggcctggtggggctctggtgggctgagggtaccgccgtggccacccgtggtttcttttttgggtacacacctgctgtctggggcgtggtgctcaaccaggccttcggcgggctactggtggctgtggttgtcaagtacgctgacaatatcctcaagggctttgccacctccctgtccattgtgctgtccactgttgcctccattcgcctctttggcttccacgtggacccattatttgcccttggcgctggactcgtcattggtgctgtctacctctacagccttccccgaggtgcagccaaagccatagcctctgcctctgcctccgcctccgggccctgcgttcaccagcagcctcccgggcagccaccaccaccgcagctgtcttcccaccgtggagacctcatcacggagccctttctgccaaagttgctcaccaaggtgaagggttcctag

SEQ ID NO: 4 - protein, 396 aa, Homo sapiens
MAAVGAGGSTAAPGPGAVSAGALEPGTASAAHRRLKYISLAVLVVQNASLILSIRYARTLPGDRFFATTAVVMAEVLKGLTCLLLLFAQKRGNVKHLVLFLHEAVLVQYVDTLKLAVPSLIYTLQNNLQYVAISNLPAATFQVTYQLKILTTALFSVLMLNRSLSRLQWASLLLLFTGVAIVQAQQAGGGGPRPLDQNPGAGLAAVVASCLSSGFAGVYFEKILKGSSGSVWLRNLQLGLFGTALGLVGLWWAEGTAVATRGFFFGYTPAVWGVVLNQAFGGLLVAVVVKYADNILKGFATSLSIVLSTVASIRLFGFHVDPLFALGAGLVIGAVYLYSLPRGAAKAIASASASASGPCVHQQPPGQPPPPQLSSHRGDLITEPFLPKLLTKVKGS

SEQ ID NO: 5 - DNA, 1689 bp, Artificial Sequence
  Description of Artificial Sequence DNA encoding a fusion protein comprising a transmembrane region of Mnn9 and catalyst region of ppGalNAc T1
atgtcactttctcttgtatcgtaccgcctaagaaagaacccgtgggttaacatttttctacctgttttggccatatttctaatatatataatttttttccagagagatcaatctctgttgactagtagaggacttcctgctggagatgttctagagccagtacaaaagcctcatgaaggtcctggagaaatggggaaaccagtcgtcattcctaaagaggatcaagaaaagatgaaagagatgtttaaaatcaatcagttcaatttaatggcaagtgagatgattgcactcaacagatctttaccagatgttaggttagaagggtgtaaaacaaaggtgtatccagataatcttcctacaacaagtgtggtgattgttttccacaatgaggcttggagcacacttctgcgaactgtccatagtgtcattaatcgctcaccaagacacatgatagaagaaattgttctagtagatgatgccagtgaaagagactttttgaaaaggcctttagagagttatgtgaaaaaactaaaagtaccagttcatgtaattcgaatggaacaacgttctggattgatcagagctagattaaaaggagctgctgtgtctaaaggccaagtgatcaccttcctggatgcccattgtgagtgtacagtgggatggctggagcctctcttggccaggatcaaacatgacaggagaacagtggtgtgtcccatcatcgatgtgatcagtgatgatacttttgagtacatggcaggctctgatatgacctatggtgggttcaactggaagctcaattttcgctggtatcctgttccccaaagagaaatggacagaaggaaaggtgatcggactcttcctgtcaggacacctaccatggcaggaggccttttttcaatagacagagattactttcaggaaattggaacatatgatgctggaatggatatttggggaggagaaaacctagaaatttcctttaggatttggcagtgtggaggaactttggaaattgttacatgctcacatgttggacatgtgtttcggaaagctacaccttacacgtttccaggaggcacagggcagattatcaataaaaataacagacgacttgcagaagtgtggatggatgaattcaagaatttcttctatataatttctccaggtgttacaaaggtagattatggagatatatcgtcaagagttggtctaagacacaaactacaatgcaaacctttttcctggtacctagagaatatatatcctgattctcaaattccacgtcactatttctcattgggagagatacgaaatgtggaaacgaatcagtgtctagataacatggctagaaaagagaatgaaaaagttggaatttttaattgccatggtatggggggtaatcaggttttctcttatactgccaacaaagaaattagaacagatgacctttgcttggatgtttccaaacttaatggcccagttacaatgctcaaatgccaccacctaaaaggcaaccaactctgggagtatgacccagtgaaattaaccctgcagcatgtgaacagtaatcagtgcctggataaagccacagaagaggatagccaggtgcccagcattagagactgcaatggaagtcggtcccagcagtggcttcttcgaaacgtcaccctgccagaaatattctga

SEQ ID NO: 6 - protein, 562 aa, Artificial Sequence
  Description of Artificial Sequence a fusion protein comprising a transmembrane region of Mnn9 and catalyst region of ppGalNAc T1
MSLSLVSYRLRKNPWVNIFLPVLAIFLIYIIFFQRDQSLLTSRGLPAGDVLEPVQKPHEGPGEMGKPVVIPKEDQEKMKEMFKINQFNLMASEMIALNRSLPDVRLEGCKTKVYPDNLPTTSVVIVFHNEAWSTLLRTVHSVINRSPRHMIEEIVLVDDASERDFLKRPLESYVKKLKVPVHVIRMEQRSGLIRARLKGAAVSKGQVITFLDAHCECTVGWLEPLLARIKHDRRTVVCPIIDVISDDTFEYMAGSDMTYGGFNWKLNFRWYPVPQREMDRRKGDRTLPVRTPTMAGGLFSIDRDYFQEIGTYDAGMDIWGGENLEISFRIWQCGGTLEIVTCSHVGHVFRKATPYTFPGGTGQIINKNNRRLAEVWMDEFKNFFYIISPGVTKVDYGDISSRVGLRHKLQCKPFSWYLENIYPDSQIPRHYFSLGEIRNVETNQCLDNMARKENEKVGIFNCHGMGGNQVFSYTANKEIRTDDLCLDVSKLNGPVTMLKCHHLKGNQLWEYDPVKLTLQHVNSNQCLDKATEEDSQVPSIRDCNGSRSQQWLLRNVTLPEIF

SEQ ID NO: 7 - DNA, 309 bp, Saccharomyces cerevisiae
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctagataaaagagctcatggtgttacttctgctccagacactagacatcaccatcaccatcactaa

SEQ ID NO: 8 - protein, 102 aa, Saccharomyces cerevisiae
MRFPSIFTAVLFAASSALAAPVNTTTEDETAQIPAEAVIGYSDLEGDFDVAVLPFSNSTNNGLLFINTTIASIAAKEEGVSLDKRAHGVTSAPDTRHHHHHH

SEQ ID NO: 9 - DNA, 1170 bp, Artificial Sequence
  Description of Artificial Sequence DNA encoding a fusion protein comprising a transmembrane region of Mnn9 and catalyst region of Drosophila beta-1,3-Gal transferase
atgtcactttctcttgtatcgtaccgcctaagaaagaacccgtgggttaacatttttctacctgttttggccatatttctaatatatataatttttttccagagagatcaatctctgttgactagttccacgccggagcgaagtgaattcatgccatacgatggccatcggcacggcgacgtgaacgatgcacatcacagccacgacatgatggagatgtccggaccggaacaggacgtgggtggacacgagcacgtgcacgagaactcgaccattgcggagcgactgtacagcgaggtgcgggtgctctgctggatcatgaccaatccgagcaaccatcagaagaaggcgcgccacgtgaagcgcacctggggcaagcgttgcaacaagctgatctttatgagctccgccaaggacgacgagctggacgcagtggctctgcccgtaggcgagggtcgcaacaacctatggggcaagacgaaggaggcctacaaatacatctacgagcatcacatcaacgacgccgactggttcctgaaggctgacgatgacacatacacgatagtggagaacatgcgatacatgctgtatccgtacagtccggaaactccagtctacttcggctgcaagttcaagccgtacgtgaaacaaggctacatgtccggcggtgccggctacgttctcagccgggaggctgttcgtcgctttgtggtcgaagccctgcccaatccgaagctgtgcaagtcggataactcgggtgctgaggacgtggagattggcaaatgtctgcagaatgtaaacgtgctcgctggggactcgcgagactcaaacgggcggggtcgcttctttccatttgtgcccgagcaccatctgattccatcgcacacggacaagaagttctggtactggcagtacatcttctacaagacggatgagggacttgactgctgctcggacaacgccatatcgttccactacgtctcccccaatcaaatgtatgtgctggattatctgatctaccatctgagaccgtacgggatcataaacacacccgatgcgttgccgaataagctagccgtgggcgaactgatgccggagatcaaggagcaggcgacggaaagcacaagtgatggggtctccaagagatccgccgagacaaagacgcaataa

SEQ ID NO: 10 - protein, 389 aa, Artificial Sequence
  Description of Artificial Sequence a fusion protein comprising a transmembrane region of Mnn9 and catalyst region of Drosophila beta-1,3-Gal transferase
MSLSLVSYRLRKNPWVNIFLPVLAIFLIYIIFFQRDQSLLTSSTPERSEFMPYDGHRHGDVNDAHHSHDMMEMSGPEQDVGGHEHVHENSTIAERLYSEVRVLCWIMTNPSNHQKKARHVKRTWGKRCNKLIFMSSAKDDELDAVALPVGEGRNNLWGKTKEAYKYIYEHHINDADWFLKADDDTYTIVENMRYMLYPYSPETPVYFGCKFKPYVKQGYMSGGAGYVLSREAVRRFVVEALPNPKLCKSDNSGAEDVEIGKCLQNVNVLAGDSRDSNGRGRFFPFVPEHHLIPSHTDKKFWYWQYIFYKTDEGLDCCSDNAISFHYVSPNQMYVLDYLIYHLRPYGIINTPDALPNKLAVGELMPEIKEQATESTSDGVSKRSAETKTQ

SEQ ID NO: 11 - DNA, 34 bp, Artificial Sequence
  Description of Artificial Sequence primer
ggaattcatgttaattaacgcaatacttgttact

SEQ ID NO: 12 - DNA, 26 bp, Artificial Sequence
  Description of Artificial Sequence primer
gctctagattattccgcactcttata

SEQ ID NO: 13 - DNA, 63 bp, Artificial Sequence
  Description of Artificial Sequence primer
ccttaattaactctgaacaaaaactcatctcagaagaggatctgaatggatctgaacaaaaac

SEQ ID NO: 14 - DNA, 62 bp, Artificial Sequence
  Description of Artificial Sequence primer
ccttaattaacgatccattcagatcctcttctgagatgagtttttgttcagatccattcaga

SEQ ID NO: 15 - DNA, 24 bp, Artificial Sequence
  Description of Artificial Sequence primer
ggaattcatggcagcggttggggc

SEQ ID NO: 16 - DNA, 35 bp, Artificial Sequence
  Description of Artificial Sequence primer
gctctagactagttaattaaggaacccttcacctt

SEQ ID NO: 17 - DNA, 28 bp, Artificial Sequence
  Description of Artificial Sequence primer
ccttaattaactacccatacgatgttcc

SEQ ID NO: 18 - DNA, 30 bp, Artificial Sequence
  Description of Artificial Sequence primer
ccttaattaatcgtcgacaccaagccgaat

SEQ ID NO: 19 - DNA, 27 bp, Artificial Sequence
  Description of Artificial Sequence primer
cgagctcatgtcactttctcttgtatc

SEQ ID NO: 20 - DNA, 29 bp, Artificial Sequence
  Description of Artificial Sequence primer
gcgtgtcgactcagaatatttctggcagg

SEQ ID NO: 21 - DNA, 23 bp, Artificial Sequence
  Description of Artificial Sequence primer
gactagtgactacaaagaccatg

SEQ ID NO: 22 - DNA, 22 bp, Artificial Sequence
  Description of Artificial Sequence primer
gactagtcttgtcatcgtcatc

SEQ ID NO: 23 - DNA, 55 bp, Artificial Sequence
  Description of Artificial Sequence primer
cgggatccggtctagataaaagagctcatggtgttacttctgctccagacactag

SEQ ID NO: 24 - DNA, 56 bp, Artificial Sequence
  Description of Artificial Sequence primer
acttctgctccagacactagacatcaccatcaccatcactaatctagaggatcccg

SEQ ID NO: 25 - DNA, 35 bp, Artificial Sequence
  Description of Artificial Sequence primer
gcttcagctgctcgagatctttgcttcaattacgc

SEQ ID NO: 26 - DNA, 35 bp, Artificial Sequence
  Description of Artificial Sequence primer
ggagaccggcctcgaccgtgcttgtagctgttagc

SEQ ID NO: 27 - DNA, 35 bp, Artificial Sequence
  Description of Artificial Sequence primer
gagtcgacctgcaggaatttcccagtactctccac

SEQ ID NO: 28 - DNA, 35 bp, Artificial Sequence
  Description of Artificial Sequence primer
agcagctgaagcttgatgtactacgcttctgttcc

SEQ ID NO: 29 - DNA, 34 bp, Artificial Sequence
  Description of Artificial Sequence primer
gcttcagctgtcgaactgtacataccgaaacgcc

SEQ ID NO: 30 - DNA, 35 bp, Artificial Sequence
  Description of Artificial Sequence primer
ggagaccggcctcgaacatgattgctggaccacgg

SEQ ID NO: 31 - DNA, 35 bp, Artificial Sequence
  Description of Artificial Sequence primer
gagtcgacctgcagggccgacaagcaagaagcatg

SEQ ID NO: 32 - DNA, 35 bp, Artificial Sequence
  Description of Artificial Sequence primer
agcagctgaagcttgggtgggctaaagggttcaag

SEQ ID NO: 33 - DNA, 35 bp, Artificial Sequence
  Description of Artificial Sequence primer
gcttcagctgctcgaagctaaaaggaacctgtccc

SEQ ID NO: 34 - DNA, 35 bp, Artificial Sequence
  Description of Artificial Sequence primer
ggagaccggcctcgacgccactctgtacggcattg

SEQ ID NO: 35 - DNA, 35 bp, Artificial Sequence
  Description of Artificial Sequence primer
gagtcgacctgcaggacttggcatggctacctacg

SEQ ID NO: 36 - DNA, 35 bp, Artificial Sequence
  Description of Artificial Sequence primer
agcagctgaagcttgtctgatcaataagcaaaccc

SEQ ID NO: 37 - DNA, 35 bp, Artificial Sequence
  Description of Artificial Sequence primer
gcttcagctgctcgagcgatacacatcctctaacc

SEQ ID NO: 38 - DNA, 35 bp, Artificial Sequence
  Description of Artificial Sequence primer
ggagaccggcctcgaagacatctgtacttctgtgg

SEQ ID NO: 39 - DNA, 35 bp, Artificial Sequence
  Description of Artificial Sequence primer
gagtcgacctgcaggcggaagttgtttctagagag

SEQ ID NO: 40 - DNA, 35 bp, Artificial Sequence
  Description of Artificial Sequence primer
agcagctgaagcttgacatggctacatcactccag

SEQ ID NO: 41 - DNA, 35 bp, Artificial Sequence
  Description of Artificial Sequence primer
gcttcagctgctcgagactatattgtccatctgcg

SEQ ID NO: 42 - DNA, 35 bp, Artificial Sequence
  Description of Artificial Sequence primer
ggagaccggcctcgagatccaccttcagcaaatgc

SEQ ID NO: 43 - DNA, 35 bp, Artificial Sequence
  Description of Artificial Sequence primer
gagtcgacctgcaggaatcatgatccatccacccg

SEQ ID NO: 44 - DNA, 35 bp, Artificial Sequence
  Description of Artificial Sequence primer
agcagctgaagcttgtccttttaccggttcaatcc

SEQ ID NO: 45 - DNA, 35 bp, Artificial Sequence
  Description of Artificial Sequence primer
gcttcagctgctcgatgggcgcaagtatattctgg

SEQ ID NO: 46 - DNA, 35 bp, Artificial Sequence
  Description of Artificial Sequence primer
ggagaccggcctcgagaaaatcccgttcccttggc

SEQ ID NO: 47 - DNA, 35 bp, Artificial Sequence
  Description of Artificial Sequence primer
gagtcgacctgcaggtctacttgggatatcgcacc

SEQ ID NO: 48 - DNA, 35 bp, Artificial Sequence
  Description of Artificial Sequence primer
agcagctgaagcttgaataacgcagctaaacgagg

SEQ ID NO: 49 - DNA, 37 bp, Artificial Sequence
  Description of Artificial Sequence primer
gctctagataaaagagctaattacaagaagcccaaac

SEQ ID NO: 50 - DNA, 25 bp, Artificial Sequence
  Description of Artificial Sequence primer
gctctagatggatcggaattcttta

SEQ ID NO: 51 - DNA, 29 bp, Artificial Sequence
  Description of Artificial Sequence primer
gctctagattcttaatcagaagagactgg

SEQ ID NO: 52 - DNA, 28 bp, Artificial Sequence
  Description of Artificial Sequence primer
cccactagttccacgccggagcgaagtg

SEQ ID NO: 53 - DNA, 29 bp, Artificial Sequence
  Description of Artificial Sequence primer
cggggtaccttattgcgtctttgtctcgg

SEQ ID NO: 54 - DNA, 37 bp, Artificial Sequence
  Description of Artificial Sequence primer
acgcgtcgacatgttaattaactctgaacaaaaactc

SEQ ID NO: 55 - DNA, 30 bp, Artificial Sequence
  Description of Artificial Sequence primer
tcccgtcgacttattccgcactcttatacc

SEQ ID NO: 56 - DNA, 34 bp, Artificial Sequence
  Description of Artificial Sequence primer
aaaaagtcgacaaaatggcagcggttggggctgg

SEQ ID NO: 57 - DNA, 34 bp, Artificial Sequence
  Description of Artificial Sequence primer
gcgtgtcgacttaccaagccgaattaacagcagc

SEQ ID NO: 58 - DNA, 29 bp, Artificial Sequence
  Description of Artificial Sequence primer
acgatgcatatgtcactttctcttgtatc

SEQ ID NO: 59 - DNA, 29 bp, Artificial Sequence
  Description of Artificial Sequence primer
acgatgcattcagaatatttctggcaggg

SEQ ID NO: 60 - DNA, 30 bp, Artificial Sequence
  Description of Artificial Sequence primer
acgcgtcgacatgagatttccttcaatttt

SEQ ID NO: 61 - DNA, 31 bp, Artificial Sequence
  Description of Artificial Sequence primer
catcaccatcaccatcactaagtcgacgcga

SEQ ID NO: 62 - DNA, 29 bp, Artificial Sequence
  Description of Artificial Sequence primer
cccactagtgccggcaggaaggaggactg

SEQ ID NO: 63 - DNA, 31 bp, Artificial Sequence
  Description of Artificial Sequence primer
cggggtaccttactgctgcaggttgagcgtg

SEQ ID NO: 64 - DNA, 29 bp, Artificial Sequence
  Description of Artificial Sequence primer
ccggaattcatgtcactttctcttgtatc

SEQ ID NO: 65 - DNA, 30 bp, Artificial Sequence
  Description of Artificial Sequence primer
acgcgtcgacttactgctgcaggttgagcg

SEQ ID NO: 66 - DNA, 28 bp, Artificial Sequence
  Description of Artificial Sequence primer
cccactagtcaaagagaagtaagtgttc

SEQ ID NO: 67 - DNA, 31 bp, Artificial Sequence
  Description of Artificial Sequence primer
cggggtaccttaatcattttggctaagtatc

SEQ ID NO: 68 - DNA, 29 bp, Artificial Sequence
  Description of Artificial Sequence primer
ccgcaattgatgtcactttctcttgtatc

SEQ ID NO: 69 - DNA, 32 bp, Artificial Sequence
  Description of Artificial Sequence primer
acgcgtcgacttaatcattttggctaagtatc

SEQ ID NO: 70 - DNA, 32 bp, Artificial Sequence
  Description of Artificial Sequence primer
ctagagctcatgagttttgtattgattttgtc

SEQ ID NO: 71 - DNA, 34 bp, Artificial Sequence
  Description of Artificial Sequence primer
tcgcgtcgactcagcgaggcagtgcagttttgac

SEQ ID NO: 72 - DNA, 28 bp, Artificial Sequence
  Description of Artificial Sequence primer
cccactagtagacacttggagcttgctg

SEQ ID NO: 73 - DNA, 27 bp, Artificial Sequence
  Description of Artificial Sequence primer
cggggtacctcagtgttttaatgtctc

SEQ ID NO: 74 - DNA, 27 bp, Artificial Sequence
  Description of Artificial Sequence primer
gactagtcaggaggagacgccagaggg

SEQ ID NO: 75 - DNA, 29 bp, Artificial Sequence
  Description of Artificial Sequence primer
ggaattcttaggagacccggtgtccccgg

SEQ ID NO: 76 - DNA, 1728 bp, Artificial Sequence
  Description of Artificial Sequence DNA encoding a fusion protein comprising a transmembrane region of Mnn9 and catalyst region of ppGalNAc T1
atgtcactttctcttgtatcgtaccgcctaagaaagaacccgtgggttaacatttttctacctgttttggccatatttctaatatatataatttttttccagagagatcaatctctgttgactagtgccggcaggaaggaggactggaatgaaattgaccccattaaaaagaaagaccttcatcacagcaatggagaagagaaagcacaaagcatggagaccctccctccagggaaagtacggtggccagactttaaccaggaagcttatgttggagggacgatggtccgctccgggcaggacccttacgcccgcaacaagttcaaccaggtggagagtgataagcttcgaatggacagagccatccctgacacccggcatgaccagtgtcagcggaagcagtggcgggtggatctgccggccaccagcgtggtgatcacgtttcacaatgaagccaggtcggccctactcaggaccgtggtcagcgtgcttaagaaaagcccgccccatctcataaaagaaatcatcttggtggatgactacagcaatgatcctgaggacggggctctcttggggaaaattgagaaagtgcgagttcttagaaatgatcgacgagaaggcctcatgcgctcacgggttcggggggccgatgctgcccaagccaaggtcctgaccttcctggacagtcactgcgagtgtaatgagcactggctggagcccctcctggaaagggtggcggaggacaggactcgggttgtgtcacccatcatcgatgtcattaatatggacaactttcagtatgtgggggcatctgctgacttgaagggcggttttgattggaacttggtattcaagtgggattacatgacgcctgagcagagaaggtcccggcaggggaacccagtcgcccctataaaaacccccatgattgctggtgggctgtttgtgatggataagttctattttgaagaactggggaagtacgacatgatgatggatgtgtggggaggagagaacctagagatctcgttccgcgtgtggcagtgtggtggcagcctggagatcatcccgtgcagccgtgtgggacacgtgttccggaagcagcacccctacacgttcccgggtggcagtggcactgtctttgcccgaaacacccgccgggcagcagaggtctggatggatgaatacaaaaatttctattatgcagcagtgccttctgctagaaacgttccttatggaaatattcagagcagattggagcttaggaagaaactcagctgcaagcctttcaaatggtaccttgaaaatgtctatccagagttaagggttccagaccatcaggatatagcttttggggccttgcagcagggaactaactgcctcgacactttgggacactttgctgatggtgtggttggagtttatgaatgtcacaatgctgggggaaaccaggaatgggccttgacgaaggagaagtcggtgaagcacatggatttgtgccttactgtggtggaccgggcaccgggctctcttataaagctgcagggctgccgagaaaatgacagcagacagaaatgggaacagatcgagggcaactccaagctgaggcacgtgggcagcaacctgtgcctggacagtcgcacggccaagagcgggggcctaagcgtggaggtgtgtggcccggccctttcgcagcagtggaagttcacgctcaacctgcagcagtaa

SEQ ID NO: 77 - protein, 575 aa, Artificial Sequence
  Description of Artificial Sequence a fusion protein comprising a transmembrane region of Mnn9 and catalyst region of ppGalNAc T2
MSLSLVSYRLRKNPWVNIFLPVLAIFLIYIIFFQRDQSLLTSAGRKEDWNEIDPIKKKDLHHSNGEEKAQSMETLPPGKVRWPDFNQEAYVGGTMVRSGQDPYARNKFNQVESDKLRMDRAIPDTRHDQCQRKQWRVDLPATSVVITFHNEARSALLRTVVSVLKKSPPHLIKEIILVDDYSNDPEDGALLGKIEKVRVLRNDRREGLMRSRVRGADAAQAKVLTFLDSHCECNEHWLEPLLERVAEDRTRVVSPIIDVINMDNFQYVGASADLKGGFDWNLVFKWDYMTPEQRRSRQGNPVAPIKTPMIAGGLFVMDKFYFEELGKYDMMMDVWGGENLEISFRVWQCGGSLEIIPCSRVGHVFRKQHPYTFPGGSGTVFARNTRRAAEVWMDEYKNFYYAAVPSARNVPYGNIQSRLELRKKLSCKPFKWYLENVYPELRVPDHQDIAFGALQQGTNCLDTLGHFADGVVGVYECHNAGGNQEWALTKEKSVKHMDLCLTVVDRAPGSLIKLQGCRENDSRQKWEQIEGNSKLRHVGSNLCLDSRTAKSGGLSVEVCGPALSQQWKFTLNLQQ

SEQ ID NO: 78 - DNA, 1917 bp, Artificial Sequence
  Description of Artificial Sequence DNA encoding a fusion protein comprising a transmembrane region of Mnn9 and catalyst region of ppGalNAc T3
atgtcactttctcttgtatcgtaccgcctaagaaagaacccgtgggttaacatttttctacctgttttggccatatttctaatatatataatttttttccagagagatcaatctctgttgactagtcaaagagaagtaagtgttcaatattccaaagaggaatcaaggatggaaaggaacatgaaaaacaaaaacaagatgttggatttaatgctagaagctgtaaacaatattaaggatgccatgccaaaaatgcaaataggagcacctgtcaggcaaaacattgatgctggtgagagaccttgtttgcaaggatattatacagcagcagaattgaagcctgtccttgaccgtccacctcaggattcaaatgcacctggtgcttctggtaaagcattcaagacaaccaatttaagtgttgaagagcaaaaggaaaaggaacgtggggaagctaaacactgctttaatgctttcgcaagtgacaggatttctttgcaccgagatcttggaccagacactcgacctcctgaatgtattgaacaaaaatttaagcgctgccctcccctgcccaccaccagtgtcataatagtttttcataatgaagcgtggtccacgttgcttagaactgtccacagtgtgctctattcttcacctgcaatactgctgaaggaaatcattttggtggatgatgctagtgtagatgagtacttacatgataaactagatgaatatgtaaaacaattttctatagtaaaaatagtcagacaaagagaaagaaaaggtctgatcactgctcggttgctaggagcaacagtcgcaacagctgaaacgctcacatttttagatgctcactgtgagtgtttctatggttggctagaacctctgttggccagaatagctgagaactacacggctgtcgtaagtccagatattgcatccatagatctgaacacgtttgaattcaacaaaccttctccttatggaagtaaccataaccgtggaaattttgactggagtctttcatttggctgggagtcgcttcctgatcatgagaagcaaagaaggaaagatgaaacctacccaattaaaacacccacttttgcaggaggacttttttccatatcaaaagaatattttgagtatattggaagctatgatgaagaaatggaaatctggggaggtgaaaatatagaaatgtctttcagagtatggcaatgtggtgggcagttggagattatgccttgctctgttgttggacatgtttttcgcagcaaaagccctcatagctttccaaaaggcactcaggtgattgctagaaaccaagttcgccttgcagaagtctggatggatgaatacaaggaaatattttataggagaaatacagatgcagcaaaaattgttaaacaaaaagcatttggtgatctttcaaaaagatttgaaataaaacaccgtcttcggtgtaaaaattttacatggtatctgaacaacatttatccagaggtgtatgtgccagaccttaatcctgttatatctggatacattaaaagcgttggtcagcctctatgtctggatgttggagaaaacaatcaaggaggcaaaccattaattatgtatacatgtcatggacttgggggaaaccagtactttgaatactctgctcaacatgaaattcggcacaacatccagaaggaattatgtcttcatgctgctcaaggtctcgttcagctgaaggcatgtacctacaaaggtcacaagacagttgtcactggagagcagatatgggagatccagaaggatcaacttctatacaatccattcttaaaaatgtgcctttcagcaaatggagagcatccaagtttagtgtcatgcaacccatcagatccactccaaaaatggatacttagccaaaatgattaa

SEQ ID NO: 79 - protein, 638 aa, Artificial Sequence
  Description of Artificial Sequence a fusion protein comprising a transmembrane region of Mnn9 and catalyst region of ppGalNAc T3
MSLSLVSYRLRKNPWVNIFLPVLAIFLIYIIFFQRDQSLLTSQREVSVQYSKEESRMERNMKNKNKMLDLMLEAVNNIKDAMPKMQIGAPVRQNIDAGERPCLQGYYTAAELKPVLDRPPQDSNAPGASGKAFKTTNLSVEEQKEKERGEAKHCFNAFASDRISLHRDLGPDTRPPECIEQKFKRCPPLPTTSVIIVFHNEAWSTLLRTVHSVLYSSPAILLKEIILVDDASVDEYLHDKLDEYVKQFSIVKIVRQRERKGLITARLLGATVATAETLTFLDAHCECFYGWLEPLLARIAENYTAVVSPDIASIDLNTFEFNKPSPYGSNHNRGNFDWSLSFGWESLPDHEKQRRKDETYPIKTPTFAGGLFSISKEYFEYIGSYDEEMEIWGGENIEMSFRVWQCGGQLEIMPCSVVGHVFRSKSPHSFPKGTQVIARNQVRLAEVWMDEYKEIFYRRNTDAAKIVKQKAFGDLSKRFEIKHRLRCKNFTWYLNNIYPEVYVPDLNPVISGYIKSVGQPLCLDVGENNQGGKPLIMYTCHGLGGNQYFEYSAQHEIRHNIQKELCLHAAQGLVQLKACTYKGHKTVVTGEQIWEIQKDQLLYNPFLKMCLSANGEHPSLVSCNPSDPLQKWILSQND

SEQ ID NO: 80 - DNA, 1356 bp, Artificial Sequence
  Description of Artificial Sequence DNA encoding a fusion protein comprising a transmembrane region of Mnn9, FLAG antigen, and catalyst region of C2GnT1
atgtcactttctcttgtatcgtaccgcctaagaaagaacccgtgggttaacatttttctacctgttttggccatatttctaatatatataatttttttccagagagatcaatctctgttgactagtgactacaaagaccatgacggtgattataaagatcatgacatcgattacaaggctgacgatgacaagactagtagacacttggagcttgctggggagaatcctagtagtgatattaattgcaccaaagttttacagggtgatgtaaatgaaatccaaaaggtaaagcttgagatcctaacagtgaaatttaaaaagcgccctcggtggacacctgacgactatataaacatgaccagtgactgttcttctttcatcaagagacgcaaatatattgtagaaccccttagtaaagaagaggcggagtttccaatagcatattctatagtggttcatcacaagattgaaatgcttgacaggctgctgagggccatctatatgcctcagaatttctattgcgttcatgtggacacaaaatccgaggattcctatttagctgcagtgatgggcatcgcttcctgttttagtaatgtctttgtggccagccgattggagagtgtggtttatgcatcgtggagccgggttcaggctgacctcaactgcatgaaggatctctatgcaatgagtgcaaactggaagtacttgataaatctttgtggtatggattttcccattaaaaccaacctagaaattgtcaggaagctcaagttgttaatgggagaaaacaacctggaaacggagaggatgccatcccataaagaagaaaggtggaagaagcggtatgaggtcgttaatggaaagctgacaaacacagggactgtcaaaatgcttcctccactcgaaacacctctcttttctggcagtgcctacttcgtggtcagtagggagtatgtggggtatgtactacagaatgaaaaaatccaaaagttgatggagtgggcacaagacacatacagccctgatgagtatctctgggccaccatccaaaggattcctgaagtcccgggctcactccctgccagccataagtatgatctatctgacatgcaagcagttgccaggtttgtcaagtggcagtactttgagggtgatgtttccaagggtgctccctacccgccctgcgatggagtccatgtgcgctcagtgtgcattttcggagctggtgacttgaactggatgctgcgcaaacaccacttgtttgccaataagtttgacgtggatgttgacctctttgccatccagtgtttggatgagcatttgagacacaaagctttggagacattaaaacactga

SEQ ID NO: 81 - protein, 451 aa, Artificial Sequence
  Description of Artificial Sequence a fusion protein comprising a transmembrane region of Mnn9, FLAG antigen, and catalyst region of C2GnT1
MSLSLVSYRLRKNPWVNIFLPVLAIFLIYIIFFQRDQSLLTSDYKDHDGDYKDHDIDYKADDDKTSRHLELAGENPSSDINCTKVLQGDVNEIQKVKLEILTVKFKKRPRWTPDDYINMTSDCSSFIKRRKYIVEPLSKEEAEFPIAYSIVVHHKIEMLDRLLRAIYMPQNFYCVHVDTKSEDSYLAAVMGIASCFSNVFVASRLESVVYASWSRVQADLNCMKDLYAMSANWKYLINLCGMDFPIKTNLEIVRKLKLLMGENNLETERMPSHKEERWKKRYEVVNGKLTNTGTVKMLPPLETPLFSGSAYFVVSREYVGYVLQNEKIQKLMEWAQDTYSPDEYLWATIQRIPEVPGSLPASHKYDLSDMQAVARFVKWQYFEGDVSKGAPYPPCDGVHVRSVCIFGAGDLNWMLRKHHLFANKFDVDVDLFAIQCLDEHLRHKALETLKH

SEQ ID NO: 82 - DNA, 1152 bp, Artificial Sequence
  Description of Artificial Sequence DNA encoding a fusion protein comprising a transmembrane region of Mnn9 and catalyst region of  3GnT6
atgtcactttctcttgtatcgtaccgcctaagaaagaacccgtgggttaacatttttctacctgttttggccatatttctaatatatataatttttttccagagagatcaatctctgttgactagtcaggaggagacgccagagggtcccaccgacgctcccgcggctgacgagccgccctcggagctcgtccccgggcccccgtgcgtggcgaacgcctcggcgaacgccacggccgacttcgagcagctgcccgcgcgcatccaggacttcctgcggtaccgccactgccgccacttcccgctgctttgggacgcaccggccaagtgcgccggcggccgaggcgtgttcctgctcctggcggtgaagtcggcgcctgagcactacgagcgacgcgagctcatccggcgcacgtgggggcaagagcgcagctacggcgggcggccagtgcgccgcctctttctattgggcaccccgggccccgaggacgaggcgcgcgcggagcggctggcggagctggtggcgctggaggcgcgcgagcacggcgacgtgctgcagtgggccttcgcggacaccttcctcaacctcacgctcaagcacctgcacttgctcgactggctggctgcacgctgcccgcacgcgcgctttctgctcagcggcgacgacgacgtgttcgtgcacaccgccaacgtagtccgcttcctgcaggcgcagccacccggccgccacctgttctccggccagctcatggagggctccgtgcccatccgcgacagctggagcaagtacttcgtgccgccgcagctcttccccgggtccgcttacccggtgtactgcagcggcggcggcttcctcctgtccggccccacggcccgggccctgcgcgcggccgcccgccacaccccgctcttccccatcgacgacgcctacatgggcatgtgtctggagcgcgccggcctggcgcccagcggccacgagggcatccggcccttcggcgtgcagctgcctggcgcacagcagtcctccttcgacccctgcatgtaccgcgagttgctgctagtgcaccgcttcgcgccctacgagatgctgctcatgtggaaggcgctgcacagccccgcgctcagctgtgaccggggacaccgggtctcctaa

SEQ ID NO: 83 - protein, 383 aa, Artificial Sequence
  Description of Artificial Sequence a fusion protein comprising a transmembrane region of Mnn9 and catalyst region of  3GnT6
MSLSLVSYRLRKNPWVNIFLPVLAIFLIYIIFFQRDQSLLTSQEETPEGPTDAPAADEPPSELVPGPPCVANASANATADFEQLPARIQDFLRYRHCRHFPLLWDAPAKCAGGRGVFLLLAVKSAPEHYERRELIRRTWGQERSYGGRPVRRLFLLGTPGPEDEARAERLAELVALEAREHGDVLQWAFADTFLNLTLKHLHLLDWLAARCPHARFLLSGDDDVFVHTANVVRFLQAQPPGRHLFSGQLMEGSVPIRDSWSKYFVPPQLFPGSAYPVYCSGGGFLLSGPTARALRAAARHTPLFPIDDAYMGMCLERAGLAPSGHEGIRPFGVQLPGAQQSSFDPCMYRELLLVHRFAPYEMLLMWKALHSPALSCDRGHRVS

SEQ ID NO: 84 - DNA, 987 bp, Artificial Sequence
  Description of Artificial Sequence full-length cDNA of MNN2-2
atgagttttgtattgattttgtcgttagtgttcggaggatgttgttccaatgtgattagtttcgagcacatggtgcaaggcagcaatataaatttgggaaatattgttacattcactcaattcgtgtctgtgacgctaattcagttgcccaatgctttggacttctctcactttccgtttaggttgcgacctagacacattcctcttaagatccatatgttagctgtgtttttgttctttaccagttcagtcgccaataacagtgtgtttaaatttgacatttccgttccgattcatattatcattagatgttcaggtaccactttgacgatgataataggttgggctgtttgtaataagaggtactccaaacttcaggtgcaatctgccatcattatgacgcttggtgcgattgtcgcatcattataccgtgacaaagaattttcaatggacagtttaaagttgaatacggattcagtgggtatgacccaaaaatctatgtttggtatctttgttgtgctagtggccactgccttgatgtcattgttgtcgttgctcaacgaatggacgtataacaagtgcgggaaacattggaaagaaactttgttctattcgcatttcttggctctaccgttgtttatgttggggtacacaaggctcagagacgaattcagagacctcttaatttcctcagactcaatggatattcctattgttaaattaccaattgctacgaaacttttcatgctaatagctaataacgtgacccagttcatttgtatcaaaggtgttaacatgctagctagtaacacggatgctttgacactttctgtcgtgcttctagtgcgtaaatttgttagtcttttactcagtgtctacatctacaagaacgtcctatccgtgactgcatacctagggaccatcaccgtgttcctgggagctggtttgtattcatatggttcggtcaaaactgcactgcctcgctga

SEQ ID NO: 85 - protein, 328 aa, Artificial Sequence
  Description of Artificial Sequence Mnn2-2
MSFVLILSLVFGGCCSNVISFEHMVQGSNINLGNIVTFTQFVSVTLIQLPNALDFSHFPFRLRPRHIPLKIHMLAVFLFFTSSVANNSVFKFDISVPIHIIIRCSGTTLTMIIGWAVCNKRYSKLQVQSAIIMTLGAIVASLYRDKEFSMDSLKLNTDSVGMTQKSMFGIFVVLVATALMSLLSLLNEWTYNKCGKHWKETLFYSHFLALPLFMLGYTRLRDEFRDLLISSDSMDIPIVKLPIATKLFMLIANNVTQFICIKGVNMLASNTDALTLSVVLLVRKFVSLLLSVYIYKNVLSVTAYLGTITVFLGAGLYSYGSVKTALPR

(19) United States
(12) Patent Application Publication
(10) Pub. No.: US 2009/0068702 A1
(43) Pub. Date: Mar. 12, 2009

(54) METHOD FOR PRODUCING A MUCIN-TYPE GLYCOPROTEIN

(75) Inventors: Yasunori CHIBA, Ibaraki (JP); Kou Amano, Ibaraki (JP); Yoshifumi Jigami, Ibaraki (JP); Kazuo Kobayashi, Gunma (JP)

Correspondence Address: FOLEY AND LARDNER LLP;SUITE 500, 3000 K STREET NW, WASHINGTON, DC, 20007, US

(73) Assignees: NATIONAL INSTITUTE OF ADVANCED INDUSTRIAL SCIENCE AND TECHNOLOGY; KIRIN BEER KABUSHIKI KAISHA

(21) Appl. No.: 11/678,678

(22) Filed: Feb. 26, 2007

(30) Foreign Application Priority Data
Mar. 3, 2006 (JP) 2006-58411

(51) Int. Cl. C12P 21/04; C12N 15/66; C07K 14/00; C12N 1/19

(52) U.S. Cl. 435/69.1; 435/471; 435/254.2; 530/395

(57) ABSTRACT

The present invention relates to a means for generating a mucin-type glycopeptide or glycoprotein on a large scale in yeast. Specifically, the invention relates to a method which comprises introducing into a yeast at least one selected from the group consisting of a gene encoding UDP-GalNAc synthetase, a gene encoding UDP-GalNAc transporter, and a gene encoding polypeptide:O-GalNAc transferase, and, if desired, a gene encoding a mucin-type glycopeptide; and producing a mucin-type glycoprotein having O-GalNAc by use of the yeast.

### CLAIM OF PRIORITY

The present application claims priority from Japanese applications JP 2006-58411 filed on Mar. 3, 2006, the contents of which are hereby incorporated by reference into this application.

### FIELD OF THE INVENTION

The present invention relates to a yeast transformant into which a mucin-type sugar chain synthesis-associated gene is introduced and to a method for producing a mucin-type glycoprotein, using the transformant.

### BACKGROUND OF THE INVENTION

Numerous studies have previously shown that a sugar chain structure binding to a protein plays an important role in the function of the protein in biological activities. A sugar chain is also called “the face of the cell”, and it is well known that the sugar chain expressed on the cell surface is involved in cellular interaction, signal transduction, development/differentiation, fertilization, cancer metastasis, or the like. Well known sugar chain modifications in mammals mainly include Asn-linked, mucin-type and proteoglycan-type modifications, each of which produces a specific sugar chain structure via a different biosynthetic pathway. There are already many findings about these synthetic pathways, some of which have been analyzed in detail.

For the Asn-linked sugar chain, an intermediate common to species from yeast to mammals, composed of 3 glucose residues, 9 mannose residues, and 2 N-acetylglucosamine residues is first produced in the endoplasmic reticulum (ER) and transferred to a particular sequence (Asn-X-Ser or Thr) in a glycoprotein by oligosaccharyltransferase. The intermediate thus transferred then undergoes processing (cleavage of glucose residues and a specific mannose residue) to synthesize a M8 high-mannose type sugar chain (Man₈GlcNAc₂) comprising 8 mannose residues and 2 N-acetylglucosamine residues. The protein containing the high-mannose type sugar chain is transported to the Golgi apparatus and undergoes various modifications; however, these modifications in the Golgi apparatus are greatly different between yeast and mammals (Kukuruzinska et al., Annu. Rev. Biochem., 56: 915-944 (1987)).

A sugar chain added to Ser or Thr also greatly varies depending on species ranging from mammals to yeast. In yeast, protein O-mannosyltransferases (Pmt proteins) transfer a first mannose residue in ER using Dol-P-Man as a substrate. In Saccharomyces cerevisiae, for example, the Pmt proteins are encoded by at least PMT1 to PMT 6 genes and form active homo- or heterodimers (Strahl et al., J. Biol. Chem., 278: 12554-12562 (2003)). In addition, the modified protein is transported from the endoplasmic reticulum to the Golgi apparatus and there subjected to addition, by mannosyltransferases in the Golgi apparatus, of a sugar chain structure in which up to 5 mannose molecules are linearly linked. The second and third mannose residues are added via α-1,2-linkage, and these transferences take place by α-1,2-mannosyltransferases encoded by the KTR1, KTR3 and KRE2 genes and the like. The fourth and fifth mannose residues are reported to be added mainly by an α-1,3-mannosyltransferase encoded by the MNN1 gene (Lussier et al., Biochi., Biophys. Acta 1426: 323-334 (1999)).

By contrast, in mammals there occur various sugar chain modifications varying depending on the organs and intracellular organelles involved. By way of example, an O-GlcNAc type sugar chain is found in the nucleus; the O-GlcNAc modification is one of the protein function-controlling mechanisms by post-translational modifications and O-GlcNAc is also an important regulatory factor in signal transduction (Wells et al., Science, 291: 2376-2378 (2001)). In the brain and nervous system, for example, mannose is added as is the case with yeast; however, GlcNAc, Gal, sialic acid, or the like is subsequently added in a Golgi apparatus to make a unique sugar chain structure (Chiba et al., J. Biol. Chem. 272, 2156-2162 (1997)). For a proteoglycan or the like, xylose is first added to the side chain of the serine residue, to which two galactose residues and a glucuronic acid residue are then added to form a core structure composed of the four sugars. Subsequently, various glycosyltransferases act thereon to generate chondroitin sulfate, dermatan sulfate, heparan sulfate, heparin and the like (Bourdon, M A, Extracellular Matrix Genes (edu. Sandell, L J, Boyd, C D) pp 157-174, Academic Press (1990)).

However, the most popularly sugar chains found in mammals are O-GalNAc type (mucin type) sugar chains in which N-acetylgalactosamine is added to Ser or Thr in mucin or the like, on which various glycosyltransferases then act to form a complex variation. Mucin is a major glycoprotein in mucus with which lumens of trachea, digestive tracts such as stomach and intestines, and gonads are covered. The core region of the mucin type sugar chain consists of N-acetylgalactosamine and a sugar directly linked thereto. Galactose or N-acetylglucosamine is linked to the hydroxyl group at the C3 of the N-acetylgalactosamine via β1-3 linkage to form a core 1 or 3 structure, respectively. N-Acetylglucosamine is linked to the core 1 or 3 structure via β1-6 linkage to form core 2 or core 4, respectively. N-Acetylgalactosamine is linked to the N-acetylgalactosamine via β1-3 linkage to form core 5; N-acetylglucosamine is linked to the N-acetylgalactosamine via β1-6 linkage to form core 6. Various core structures are produced by these combinations (FIG. 1). A stem region extending in repeated lactosamine sequences from each core is composed of the disaccharides Gal(β1-3)GlcNAc (type 1) and Gal(β1-4)GlcNAc (type 2). The type 1 and 2 sugar chains branch by the linking of N-acetylglucosamine to the galactose via β1-6 and β1-3 linkages. The sugar chain of the stem region terminates with a peripheral sugar, typically alpha-linked galactose, N-acetylgalactosamine, fucose or sialic acid.

Among the sugar chain antigens found in glycolipids and mucins of cancer cell membranes and in mucins in the serum of cancer patients are sugar chain antigens having antigenicity at peripheral sugar chains thereof as typified by blood group antigens and sugar chain antigens having antigenicity at core structures thereof such as Tn, T, and sialyl Tn and sialyl T in which sialic acid is linked to Tn and T, respectively. The cancer-associated sugar chain antigens examined in detail both structurally and functionally are a sialyl Lewis-A antigen and its isomer, a sialyl Lewis-X antigen, which are also well known to be ligands for a selectin family which is considered to be involved in cancer cell metastasis.

Many clinical studies have shown a clear correlation between the presence of these sugar chain antigens and the long-term survival rate. The results of these studies strongly suggest that particular structures in mucin sugar chains can be cancer markers and are involved in the metastatic ability of cancer cells.

When cancer cells make hematogenous metastasis, it is considered, most of the cancer cells entering the bloodstream die rapidly and 0.01% or less thereof can get out of the blood vessels and again grow in secondary organs. In the hematogenous metastasis, cancer cells are believed to induce platelet aggregation to allow the platelets to form aggregates, thereby escaping the attack thereof. In fact, many cancer cells are known to induce platelet aggregation; cancer cells having a higher metastatic ability tend to have a higher ability to aggregate platelets. Platelet aggregation by cancer cells is thought to facilitate the trapping of cancer cells in the bloodstream by microvessels of secondary organs. Platelet aggregation by cancer cells is thus suggested to be important for metastasis formation, but the platelet-aggregating factor induced by cancer cells has not previously been identified. In recent years, a protein called aggrus/podoplanin expressed on the surface of cancer cells has been reported to be a platelet-aggregating factor (Kato et al., J. Biol. Chem. 278: 51599-51605 (2003)). In addition, the platelet-aggregating activity has been reported to disappear by the deletion of sialic acid in a core 1 structure added to aggrus (Kaneko et al., J. Biol. Chem., 279: 38838-38843 (2004)).

These findings suggest that a glycoprotein having a particular mucin-type sugar chain structure can be allowed to act competitively in the blood as one of methods for suppressing cancer metastasis and can be applied to “immunotherapy”, which enhances immunologic competence, by supplying the glycoprotein as an antigen. In addition, an antibody drug directed against cancer cells can be expected to be developed using a specific sugar chain antibody obtained employing as an antigen a glycoprotein containing a mucin-type sugar chain structure characteristic of cancer cells. For realizing these applications, it is required to produce mucin-type glycoproteins having uniform structures on a large scale.

A mucin-type sugar chain per se can be produced on a large scale by organochemical synthesis. An O-GalNAc-containing peptide can be also produced by a chemical synthesis method of peptide using GalNAc-Ser or GalNAc-Thr as a donor. However, a method for chemically synthesizing a high molecular protein has not been established. If O-GalNAc-containing peptides could be joined, it would be an extremely expensive production method and there would be also a low possibility that the higher-order structure of protein itself would be reproduced. In addition, the production of a mucin-type glycoprotein using animal cells or the like has many problems in view of the non-uniformity of production and product, the cost of culture, the contamination of virus, and the time taken for obtaining stable producing cells. Particularly, the mammalian cell originally has numerous glycosyltransferases for producing various sugar chain structures. Thus, for producing a uniform mucin-type sugar chain structure, it is required to obtain its variant having reduced glycosyltransferase activities or to control the glycosyltransferase activities using RNAi or the like.

The present inventors have previously reported methods for synthesizing, in yeast, Asn-linked high mannose-type sugar chains, acidic sugar chains, and O-fucose-containing sugar chains by use of gene introduction or various variants (JP Patent Publication (Kokai) No. 2005-328841A, WO03/091431, JP Patent Publication (Kokai) No. 2002-369692A, WO01/014522, JP Patent Publication (Kokai) No. 10-155495A (1998), JP Patent Publication (Kokai) No. 09-266792A (1997), JP Patent Publication (Kokai) No. 09-135689A (1997), JP Patent Publication (Kokai) No. 06-277086A (1994), WO02/031159, and JP Patent Publication (Kokai) No. 2005-328841A), but have not reported a method for expressing mucin-type sugar chains in yeast. Researchers in Japan and overseas other than the present inventors have also reported methods for synthesizing Asn-liked, hybrid- and complex-type sugar chains in yeast by use of gene introduction or various variants (WO02/000856, WO02/000879, WO03/056914, and WO04/003194), but again have not reported a method for expressing mucin-type sugar chains in yeast.

### SUMMARY OF THE INVENTION

With the foregoing current circumstances in view, an object of the present invention is to provide a means for producing a mucin-type glycopeptide or glycoprotein on a large scale in yeast.

As a result of intensive studies for solving the above-described problems, the present inventors have demonstrated that a mucin-type glycoprotein can be efficiently produced in yeast by newly introducing into the yeast a mucin-type glycoprotein synthesis-associated gene originally absent in yeast, thereby accomplishing the present invention.

Thus, the present invention provides a method for producing a transformed yeast for generating a mucin-type glycoprotein having O-GalNAc, comprising the step of introducing into a yeast a gene encoding UDP-GalNAc synthetase, a gene encoding a GalNAc transporter, or a gene encoding polypeptide O-GalNAc transferase.

The above method may comprise both steps of introducing the gene encoding UDP-GalNAc synthetase into the yeast and introducing the gene encoding a GalNAc transporter into the yeast.

Alternatively, the method may comprise both steps of introducing the gene encoding UDP-GalNAc synthetase into the yeast and introducing the gene encoding polypeptide:O-GalNAc transferase into the yeast.

Alternatively, the method may comprise both steps of introducing the gene encoding a GalNAc transporter and the gene encoding polypeptide:O-GalNAc transferase into the yeast.

The method may also comprise all steps of introducing the gene encoding UDP-GalNAc synthetase into the yeast, introducing the gene encoding a GalNAc transporter into the yeast, and the gene encoding polypeptide:O-GalNAc transferase into the yeast.

In addition, the method may comprise the step of further introducing a gene encoding the mucin-type glycoprotein into the yeast obtained.

According to the method of the present invention, the above polypeptide O-GalNAc transferase preferably has the transmembrane region of a protein localized in the Golgi apparatus of yeast; the above gene encoding the mucin-type glycoprotein preferably has a secretion signal sequence derived from yeast.

The present invention also provides a method for producing a transformed yeast for generating a mucin-type glycoprotein having a core 1 structure. This method comprises the step of further introducing a gene encoding core 1 β1-3 Gal transferase into the yeast for generating a mucin-type glycoprotein having O-GalNAc obtained by the above method.

The core 1 β1-3 Gal transferase preferably has the transmembrane region of a protein localized in the Golgi apparatus of yeast.

Further, the above method may comprise the step of introducing a gene encoding UDP-Gal synthetase into the yeast when the UDP-GalNAc synthetase has no UDP-Gal synthetic activity and may comprise the step of introducing a gene encoding UDP-Gal transporter into the yeast when the UDP-GalNAc transporter has no UDP-Gal transport activity.

The present invention also provides a method for producing a transformed yeast for generating a mucin-type glycoprotein having a core 2 structure. This method comprises the step of further introducing a gene encoding core 2 β-1,6-GlcNAc transferase into the yeast for generating a mucin-type glycoprotein having O-GalNAc obtained by the above method.

The present invention also provides a method for producing a transformed yeast for generating a mucin-type glycoprotein having a core 3 structure. This method comprises the step of further introducing a gene encoding core 3 β-1,3-GlcNAc transferase into the yeast for generating a mucin-type glycoprotein having O-GalNAc obtained by the above method.

The above method may comprise the step of further introducing a gene encoding the mucin-type glycoprotein into the yeast obtained.

The above gene encoding the mucin-type glycoprotein preferably has a secretion signal sequence derived from yeast.

The yeast usable in the present invention is preferably a methanol-utilizing yeast such as, for example, Ogataea minuta. Saccharomyces cerevisiae can be also preferably used.

The present invention also provides a transformed yeast obtained by any one of the above methods.

The present invention also provides a method for producing a mucin-type glycoprotein, comprising the steps of culturing the above yeast and collecting the mucin-type glycoprotein from the culture. This method may further comprise the step of exposure to a compound inhibiting the activity of an O-mannosylation enzyme.

The present invention also provides a mucin-type glycoprotein obtained by the above method.

In place of the exposure to the compound inhibiting the activity of an O-mannosylation enzyme, the method may comprise the step of disrupting the enzyme having O-mannosylation activity in the yeast during the production of the transformed yeast. The present invention also provides a yeast produced by such a method, a mucin-type glycoprotein prepared using the yeast, and a method for preparing the glycoprotein.

According to the present invention, a mucin-type glycoprotein can be efficiently and inexpensively produced on a large scale by use of yeast. The technique of the present invention can greatly contribute to the development of various therapies such as a cancer immunotherapy using a mucin-type glycopeptide as an antigen, and the drug development including the preparation of an antibody to a mucin-type glycoprotein-specific antigen present on the surface of cancer cells and the application of the antibody to an antibody drug.

### BRIEF DESCRIPTION OF THE DRAWINGS

FIG. 1 is a schematic diagram of the biosynthetic pathways of mucin-type sugar chains having various core structures;

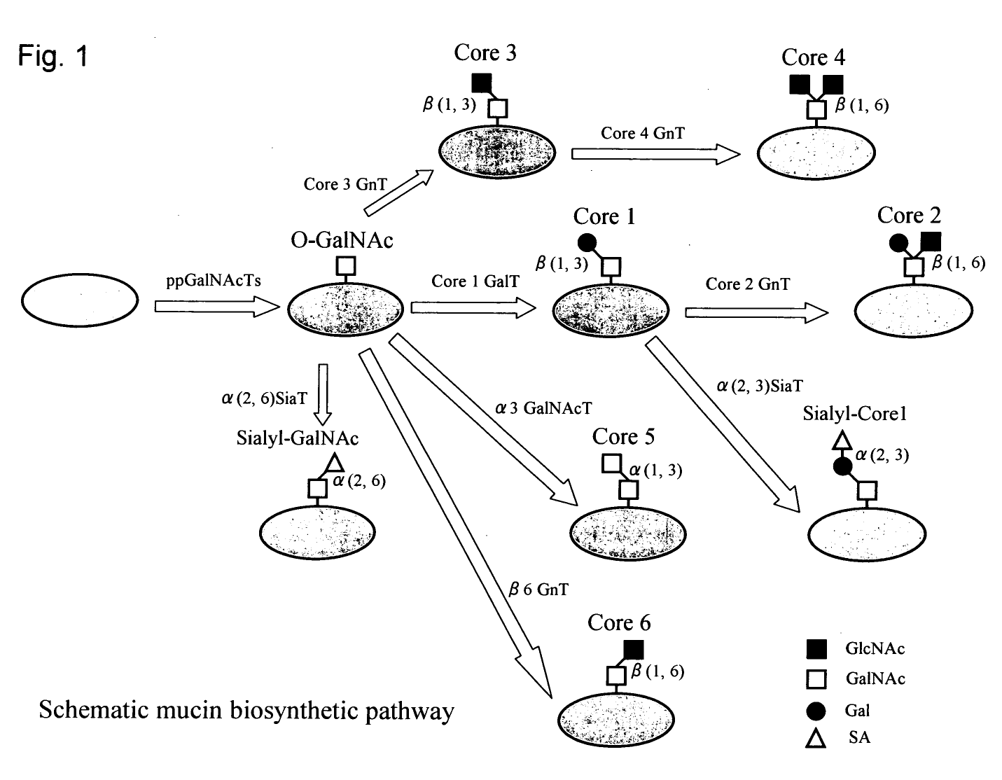

FIG. 2 is a schematic diagram of the biosynthetic pathway of a mucin-type sugar chain;

FIG. 3 is the results of checking the biosynthesis of UDP-Gal and UDP-GalNAc in the S. cerevisiae strains KAY-1 and W303-1A by use of reversed-phase HPLC;

FIG. 4 is the results of checking gene expression in the S. cerevisiae strains KAY-4 and W303-1A by use of Western blotting;

FIG. 5 is the reversed-phase HPLC analysis of purified products derived from the S. cerevisiae strains KAY-4 and KAY-5;

FIG. 6 is the mass spectrometric analysis of MUC1a peptides derived from the S. cerevisiae strains KAY-4 and KAY-5 by use of MALDI TOF-MS;

FIG. 7 is the analysis of MUC1a peptide derived from the S. cerevisiae strain KAY-4 by use of a lectin array;

FIG. 8 is the results of purity test of MUC1a peptide derived from a mucin-type glycopeptide-producing S. cerevisiae strain in the presence of an inhibitor (a rhodamine-3-acetic acid derivative);

FIG. 9 is the results of checking the addition of GalNac to Cm10 in the S. cerevisiae strains KAY-27 and KAY-29 by use of lectin blotting;

FIG. 10 is the results of checking the expression of Corel GalT derived from Drosophila in the S. cerevisiae strains KAY-30 and W303-1A by use of Western blotting;

FIG. 11 is the reversed-phase HPLC analysis of a purified product derived from the S. cerevisiae strain KAY-13;

FIG. 12 is the mass spectrometric analysis of MUC1a peptide derived from the S. cerevisiae strain KAY-13 by use of MALDI TOF-MS;

FIG. 13 is the sugar chain structure analysis of a purified preparation (mass spectrometric analysis: 24 min. peak) derived from the S. cerevisiae strain KAY-13 by use of treatment with β-galactosidase;

FIG. 14 is the analysis of MUC1a peptide derive from the S. cerevisiae strain KAY-13 by use of a lectin array;

FIG. 15 is the reversed-phase HPLC analysis of a purified product derived from the O. minuta strain KAM-3;

FIG. 16 is the mass spectrometric analysis of MUC1a peptide derived from the O. minuta strain KAM-3 by use of MALDI TOF-MS;

### DETAILED DESCRIPTION OF THE PREFERRED EMBODIMENTS

### 1. Definition

For the purpose of the present invention, “mucin-type sugar chain” refers to a sugar chain comprising an O-GalNAc structure in which GalNAc (N-acetylgalactosamine) is added to the hydroxyl group of Ser or Thr of a protein or peptide (a mucin-type sugar chain having O-GalNAc is sometimes generally referred to as Tn-antigen). Examples of the mucin-type sugar chain include the following. A sugar chain structure in which galactose or N-acetylglucosamine is β1-3 linked to the hydroxyl group at the C3 of N-acetylgalactosamine is called core 1 (Ser/Thr-O-α1GalNAc3-β1Gal) or core 3 (Ser/Thr-O-α1GalNAc3-β1GlcNAc), respectively. A structure in which glucosamine is further β1-6 linked to the core 1 or 3 is called core 2 or 4, respectively; a structure in which N-acetylgalactosamine is α1-3 linked to the hydroxyl group at the C3 of N-acetylgalactosamine is called core 5; and a structure in which N-acetylglucosamine is β1-6 linked to the hydroxyl group at the C3 of N-acetylgalactosamine is called core 6. On the other hand, a stem region extending in repeated lactosamine sequences from each core is composed of the disaccharide structures Gal(β1-3)GlcNAc (type 1) and Gal(β1-4)GlcNAc (type 2). The type 1 and 2 sugar chains branch by the further linking of N-acetylglucosamine to the galactose via β1-6 and β1-3 linkages, and terminate with typically α-linked galactose, N-acetylgalactosamine, fucose or sialic acid.

For the purpose of the present invention, “mucin-type glycoprotein” refers to a protein or peptide having the above mucin-type sugar chain. The mucin-type glycoprotein is also described as a mucin-type glycoprotein/glycopeptide or a mucin-type sugar chain-added protein/peptide. Examples of the mucin-type glycoprotein include MUC1a (SEQ ID NO: 7), MUC1 (Genbank: P15941), MUC3 (Genbank: AF016694), MUC4 (Genbank: AJ000281), MUC5B (Genbank: AF107890), MUC6 (Genbank: AK096772), MUC7 (Genbank: BC025688), MUC8 (Genbank: AF335495), MUC9 (Lapensee et al., Fertil. Steril. 68: 702-708 (1997)), MUC11 (Genbank: AF 147791) and functional fragments of these mucins, antibodies to the mucins and functional fragments thereof, and erythropoietin. The functional fragment of the mucin is a portion (partial fragment) of the mucin sequence and refers to the full length, or a partial sequence having antigenicity, of a repeated sequence (tandem repeats) which is a constituent of mucin. The functional fragment of the antibody refers to a portion (partial fragment) of the antibody, retaining one or more effects of the antibody on an antigen; specific examples thereof include F(ab′)2, Fab′, Fab, Fv, disulfide-bonded Fv, single-chain Fv (scFv), and their polymers (D. J. King., Applications and Engineering of Monoclonal Antibodies., 1998 T.J. International Ltd).

For the purpose of the present invention, “mucin-type glycoprotein synthesis-associated gene” is a generic term applied to a gene involved in the synthesis of a mucin-type glycoprotein in the yeast cell. By way of example, the gene is a gene required in the processes of: synthesizing a sugar donor necessary in producing a mucin-type sugar chain: UDP-N-acetylgalactosamine (UDP-GalNAc), UDP-galactose (UDP-Gal), UDP-N-acetylglucosamine (UDP-GlcNAc), or GDP-fucose (GDP-Fuc); transporting the donor to the place of transglycosylation; conducting transglycosylation to synthesize a mucin-type glycoprotein; and secreting the glycoprotein outside the cells (Hiroshi Nakada, Muchin oyobi Muchingata Tosa no Tayosei to Sonoigi (Diversity of Mucins and Mucin-type Sugar Chains and its Significance), “Posuto-Genomu Jidaino Tosa Seibutsugakuga Wakaru (Sugar Chain Biology in Post-genome Era Can be understood)”, (ed. Naoyuki Taniguchi), Yodosha Co., Ltd, pp. 37-44).

Examples of the mucin-type glycoprotein synthesis-associated gene include genes for UDP-GalNAc synthetase, UDP-GalNAc transporter, polypeptide:O-GalNAc transferase, core 1 β1-3 Gal transferase, core 2 β-1,6-GlcNAc transferase, and core 3 β-1,3-GlcNAc transferase.

For the purpose of the present invention, “UDP-GalNAc synthetase” refers to a protein having the activity of converting UDP-acetylglucosamine (UDP-GlcNAc) to UDP-acetylgalactosamine (UDP-GalNAc) (by “UDP-GalNAc synthetic activity” is sometimes herein meant the activity of converting UDP-acetylglucosamine (UDP-GlcNAc) to UDP-acetylgalactosamine (UDP-GalNAc)). Examples of the UDP-GalNAc synthetase include human GalE, porcine GalE, Bacillus subtilis GalE (Genbank: P55180), Yersinia enterocolitica GalE, Escherichia coli GNE, Streptococcus gordonii Ga/E2, and Pseudomonas aeruginosa GalE (Schulz et al., J. Biol. Chem., 279: 32796-32803 (2004)). In this respect, some UDP-GalNAc synthetases also have the activity of converting UDP-glucose (UDP-Glc) to UDP-galactose (UDP-Gal) (by “UDP-Gal synthetic activity” is sometimes herein meant the activity of converting UDP-glucose (UDP-Glc) to UDP-galactose (UDP-Gal)) (a protein having UDP-Gal synthetic activity is sometimes herein referred to as “UDP-Gal synthetase”, which is not limited to UDP-GalNAc synthetases). Examples of such a protein include human GalE, porcine GalE, Bacillus subtilis GalE (Genbank: P55180), Yersinia enterocolitica GalE, Escherichia coli GNE, and Streptococcus gordonii GalE2.

For the purpose of the present invention, “UDP-GalNAc transporter” refers to a protein having the ability to transport UDP-GalNAc into the lumen of the Golgi apparatus (by “UDP-GalNAc transport activity” is sometimes herein meant the ability to transport UDP-GalNAc into the lumen of the Golgi apparatus). Examples of the UDP-GalNAc transporter include human UDP-Gal transporter (UGT2) (Genbank: D87969) and human UDP-GlcA/UDP-GalNAc (UGTrel7) (FEBS Lett., 495: 87-93 (2001)). In this respect, some UDP-GalNAc transporters also have the ability to transport UDP-Gal into the lumen of the Golgi apparatus (by “UDP-Gal transport activity” is sometimes herein meant the ability to transport UDP-Gal into the lumen of the Golgi apparatus) (a protein having UDP-Gal transport activity is sometimes herein referred to as “UDP-Gal transporter”, which is not limited to UDP-GalNAc transporters). Examples of such a protein include human UDP-Gal transporter (UGT2) (Genbank: D87969) and yeast-derived UDP-Gal transporter (Glycobiology, 9: 133-141 (1999)).

For the purpose of the present invention, “polypeptide:O-GalNAc transferase” refers to a protein having the activity of transferring GalNAc to Ser or Thr of a protein or peptide by use of UDP-GalNAc as a sugar donor. Examples of the polypeptide:O-GalNAc transferase include human ppGalNAcT-1 (Genbank: X85018), human ppGalNAcT-2, human ppGalNAcT-3, human ppGalNAcT-4 to 13, mouse ppGalNAcT-1 (Ten Hagen et al., Glycobiology: 13, 1R-16R (2003)), and Drosophila ppGalNAcT (Tian & Ten Hagen, Glycobiology: 16, 83-95 (2006)).

For the purpose of the present invention, “core 1 β1-3 Gal transferase” refers to a protein having the activity of transferring Gal, via β1-3 linkage, to the GalNAc residue of a mucin-type glycoprotein having GalNAc added, by use of UDP-Gal as a sugar donor to form a mucin-type sugar chain of a core 1 structure (the mucin-type sugar chain of a core 1 structure is sometimes generally called T-antigen). Examples of the core 1 β1-3 Gal transferase include human β3-GalT-1 (Genbank: E07739, AF117222), human β3-GalT-2 (Genbank: CAA75245, CAA75344), and Drosophila β-1,3-GalT (Genbank: CG9520). The core 1 β1-3 Gal transferase is also called core 1 synthetase or core 1 GalT.

For the purpose of the present invention, “core 2 β-1,6-GlcNAc transferase” refers to a protein which transfers GlcNAc, via β-1,6 linkage, to the GalNAc residue of a mucin-type glycoprotein having a mucin-type sugar chain of a core 1 structure by use of UDP-GlcNAc as a sugar donor to form a mucin-type sugar chain of a core 2 structure. Examples of the core 2 β-1,6-GlcNAc transferase include human β6-GnT-1 (Genbank: M97347), human β6-GnT-2 (Genbank: AF03865, AF102542), and human P6-GnT-3 (Genbank: AF132035). The core 2 β-1,6-GlcNAc transferase is also called core 2 synthetase or core 2 GnT.

For the purpose of the present invention, “core 3 β-1,3-GlcNAc transferase” refers to a protein which transfers GlcNAc, via β1-3 linkage, to the GalNAc residue of a mucin-type glycoprotein having GalNAc added, by use of UDP-GlcNAc as a sugar donor to form a mucin-type sugar chain of a core 3 structure. Examples of the core 3 β-1,3-GlcNAc transferase include human β3-GnT-6 (Genbank: AW182889). The core 3 β-1,3-GlcNAc transferase is also called core 3 synthetase or core 3 GnT.

For the purpose of the present invention, “Protein-O-Mannosyltransferase (or PMT)” refers to a protein having the activity of transferring mannose (Man) to Ser of Thr of a protein or peptide in ER by use of Dol-P-Man as a sugar donor (by “O-mannosylation activity” is sometimes herein meant the activity of transferring mannose (Man) to Ser of Thr of a protein or peptide; a protein having O-mannosylation activity is sometimes herein called “O-mannosylation enzyme”). Examples of PMT include S. cerevisiae PMT1 (gene) (Genbank: L19169), PMT2 (gene) (Genbank: L05146), PMT3 (gene) (Genbank: X83797), PMT4 (gene) (Genbank: X83798), PMT5 (gene) (Genbank: X95644), and PMT6 (gene) (Genbank: Z72984), or O. minuta PMTs 1 to 5.

For the purpose of the present invention, “methanol-utilizing yeast” refers to a group of yeasts which are viable by use of methanol as the only carbon source; examples thereof include Pichia pastoris, Ogataea minuta, Pichia methanolica, and Hansenula polymorpha (Pichia angusta).

### 2. Mucin-Type Glycoprotein Synthesis-Associated Genes

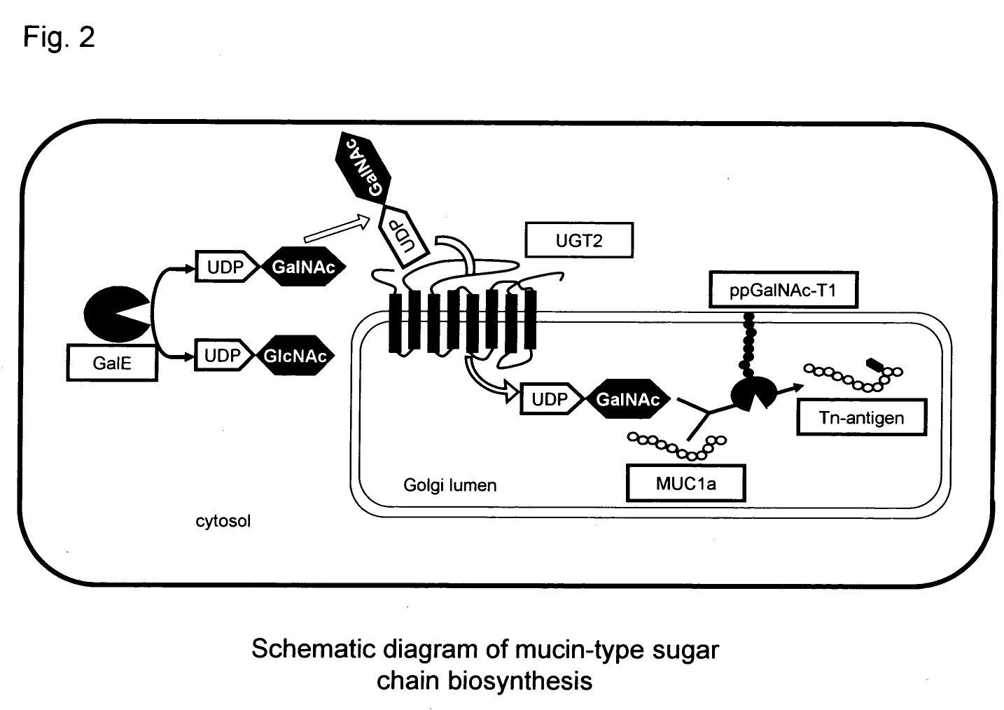

FIG. 2 shows the synthetic process of a mucin-type glycoprotein in yeast when mucin-type glycoprotein synthesis-associated genes of the present invention are introduced into the yeast. The Tn-antigen in the figure means a GalNAc structure added to Ser/Thr on the peptide.

### (1) A Gene Encoding UDP-GalNAc Synthetase

For producing a mucin-type glycoprotein in yeast, it is first necessary to synthesize UDP-N-acetylgalactosamine (UDP-GalNAc) in the yeast cytoplasm. For this purpose, a gene encoding UDP-GalNAc synthetase is introduced into the yeast. In this respect, some genes encoding UDP-GalNAc synthetase also have UDP-galactose (UDP-Gal) synthetic activity.

In Examples of the present invention, a gene for Bacillus subtilis GalE was introduced as the gene encoding UDP-GalNAc synthetase; the nucleotide sequence and the amino acid sequence are shown in SEQ ID NOS: 1 and 2, respectively, in Sequence Listing.

In this respect, galactose (Gal) and N-acetylgalactosamine (GalNAc) can be also incorporated into cells, followed by condensing these sugars with UDP to produce UDP-Gal and UDP-GalNAc without converting the UDP-Glc and UDP-GlcNAc generated by the yeast to UDP-Gal and UDP-GalNAc, respectively. Thus, genes encoding proteins having the condensation activities may be also introduced into the yeast in place of the gene encoding UDP-GalNAc synthetase and the gene encoding UDP-Gal synthetase. Examples of the enzymes condensing UDP with the sugars can include GAL7 (UTP: galactose-1-phosphate uridylyltransferase) (Genbank: NC_{—}001134). The method of the present invention does not preclude the use of such enzymes condensing UDP with the sugars.

In this respect, a gene for UDP-GlcNAc synthetase does not particularly need to be introduced because UDP-GlcNAc is synthesized in yeast for the synthesis of cell wall chitin, but can also be externally introduced as will be described in Examples of the present application. The expression of the gene is also known to be enhancable by introducing GFA1 (Genbank: Z28104), GNA1 (Genbank: AB017626), PCM1 (Genbank: X75816), QR11 (Genbank: X79380), or the like which are involved in the synthesis of UDP-GlcNAc in budding yeast.

### (2) A Gene Encoding UDP-GalNAc Transporter

It is then necessary to transport the UDP-GalNAc accumulated in the yeast cytoplasm to a suitable organelle serving as the place of transglycosylation. For this purpose, a gene encoding UDP-GalNAc transporter is introduced, into the yeast, as a gene imparting to the yeast the ability to transport these sugar nucleotides into the lumen of the Golgi apparatus serving as the place of glycoprotein synthesis. In this respect, some UDP-GalNAc transporters also have the ability to transport UDP-galactose (UDP-Gal).

By way of example, it has been reported from the results of in vitro activity determination that the product of UGT2 gene has the ability to transport not only UDP-Gal but also UDP-GalNAc (Segawa et al., Eur. J. Biochem., 269: 128-138 (2002)). Thus, the UGT2 gene is preferable in that it can be introduced to at once impart to the yeast the ability to transport both UDP-GalNAc and UDP-Gal to the lumen of a Golgi apparatus. The nucleotide sequence for and amino acid sequence of UGT2 are shown in SEQ ID NOS: 3 and 4, respectively, in Sequence Listing.

In addition, the transporter activity of transporting UDP-Gal has been found in yeast (Roy et al., J. Biol. Chem., 273: 2583-2590 (1998)); it is also possible to make effective use of the activity, but the transporter can be externally introduced as will be described in Examples of the present application.

Likewise, UDP-GlcNAc transporter is also required to be expressed in yeast; the expression can be conducted using a method as described in WO02/031159 or WO02/000879. Specifically, the expression can be achieved in yeast by introducing human UGTrel2 gene (Genbank: ABO21981), Kluyveromyces lactis MNN2 (Genbank: AF 106080) gene, or the like.

(3) A Gene Encoding Polypeptide:O-GalNAc Transferase (or ppGalNAcT)

In addition, to make use of UDP-GalNAc as a sugar donor, a gene encoding polypeptide:O-GalNAc transferase is introduced into the yeast.

A little less than 20 types of polypeptide:O-GalNAc transferases are currently known in mammals, but none of these transferases have been so far identified in yeast. For allowing polypeptide:O-GalNAc transferase to function successfully in the lumen of the yeast Golgi apparatus, it is preferable, for example, to make a gene design so that the gene contains a nucleotide sequence encoding a protein in which the transmembrane region of a protein localized in the yeast Golgi apparatus is fused to a region comprising the catalytic domain of the polypeptide:O-GalNAc transferase. In Examples of the present invention, human ppGalNAcT-1 was selected as the polypeptide:O-GalNAc transferase, and Mnn9 protein, as the protein localized in the Golgi apparatus. The nucleotide sequence and amino acid sequence of a gene encoding a protein in which the transmembrane region of Mnn9 is fused to the catalytic domain of ppGalNAc T1 are shown in SEQ ID NOS: 5 and 6, respectively, in Sequence Listing; the nucleotide sequence and amino acid sequence of a gene encoding a protein in which the transmembrane region of Mnn9 is fused to the catalytic domain of ppGalNAc T2 are shown in SEQ ID NOS: 76 and 77, respectively in Sequence Listing; and the nucleotide sequence and amino acid sequence of a gene encoding a protein in which the transmembrane region of Mnn9 is fused to the catalytic domain of ppGalNAc T3 are shown in SEQ ID NOS: 78 and 79, respectively in Sequence Listing.

### (4) a Gene Encoding a Mucin-Type Glycoprotein

In Examples of the present invention, the gene encoding MUC1a peptide as a mucin-type glycoprotein was introduced.

It is desirable to further add a yeast-derived secretion signal sequence to the gene encoding the mucin-type glycoprotein. This allows, for example, the mucin-type sugar-added protein synthesized on the ribosome to be carried from the lumens of the endoplasmic reticulum and Golgi apparatus through the secretion pathway into a medium, and results in enabling the mucin-type sugar-added protein to be produced with high efficiency.

As the yeast-derived secretion signal there may be used a conventional known or well-known one; examples thereof include, but not limited to, signal sequences derived from the α-factor precursor (Posuto Shikuensu Tanpakushitsu Jikkenho 2. Shiryo Choseiho (Post-sequence Protein Experimental Methods: 2. Sample-preparing Methods), ed. Tairo Ohshima, Tokyo Kagaku Dozin Co., Ltd., pp 45-55), invertase (SUC2) (GenBank Accession No. V01311), acid phosphatase (PHO5) (GenBank Accession No. Z35962), α-1,2mannosyltransferase (KRE2) (GenBank Accession No. X62647) and KAR2 (GenBank Accession No. M25064) gene products of budding yeast. When the α-factor precursor is used, the mucin-type glycoprotein is expressed in the form of a protein in which the mucin-type sugar chain-added protein is fused downstream of the yeast α-factor preprosequence; however, only the intended mucin-type glycoprotein is obtained because the preprosequence in the fusion is removed by signal peptidase or Kex2 protein when secreted outside the yeast cell. The nucleotide sequence of MUC1a peptide and corresponding amino acid sequence are shown in the region from nucleotide 253 to 288 in SEQ ID NO: 7 and in the region from amino acid 85 to 96 (downstream of the α-factor preprosequence and before the histidine tag) in SEQ ID NO: 8, respectively, in Sequence Listing.

(5) A gene Encoding Core 1 β1-3 Gal transferase

Core 1 β1-3 Gal transferase has not been identified so far in yeast.

The transfer reaction of a sugar chain occurs mainly in the lumen of the Golgi apparatus. For expressing the above glycosyltransferase in the yeast Golgi apparatus, it is more preferable to add thereto the transmembrane region of a protein localized in the yeast Golgi apparatus. As the protein localized in the yeast Golgi apparatus there may be used a conventional known or well-known one; examples thereof include, but not limited to, the budding yeast glycosyltransferases Och1 protein (GenBank Accession No. D11095), Mnn9 protein (GenBank Accession No. L23752) and Kre2 protein (GenBank Accession No. X62647). For allowing core 1 β1-3 Gal transferase to function successfully in the lumen of the yeast Golgi apparatus, a gene design is made, for example, so that the gene therefor comprise a nucleotide sequence encoding a protein in which the transmembrane region of a protein localized in the yeast Golgi apparatus is fused to a region comprising the catalytic domain of the core 1 β1-3 Gal transferase. In Examples of the present invention, Drosophila β-1,3-Gal transferase was selected as the core 1 β1-3 Gal transferase, and Mnn9 protein, as the protein localized in the yeast Golgi apparatus. The nucleotide sequence and amino acid sequence of a gene encoding a protein in which the transmembrane region of Mnn9 is fused to the catalytic domain of Drosophila β-1,3-Gal transferase are shown in SEQ ID NOS: 9 and 10, respectively, in Sequence Listing.

### (6) A Gene Encoding Core 2 β-1,6-GlcNAc Transferase

Core 2 β-1,6-GlcNAc transferase has not been identified so far in yeast.

The transfer reaction of a sugar chain occurs mainly in the lumen of the Golgi apparatus. For expressing the above glycosyltransferase in the yeast Golgi apparatus, it is more preferable to add thereto the transmembrane region of a protein localized in the yeast Golgi apparatus. As the protein localized in the yeast Golgi apparatus there may be used a conventional known or well-known one; examples thereof include, but not limited to, the budding yeast glycosyltransferases Och1 protein (GenBank Accession No. D11095), Mnn9 protein (GenBank Accession No. L23752) and Kre2 protein (GenBank Accession No. X62647). For allowing core 2 β-1,6-GlcNAc transferase to function successfully in the lumen of the yeast Golgi apparatus, a gene design is made, for example, so that the gene therefor comprises a nucleotide sequence encoding a protein in which the transmembrane region of a protein localized in the yeast Golgi apparatus is fused to a region comprising the catalytic domain of the core 2 β-1,6-GlcNAc transferase. By way of example, human β6-GnT-2 may be selected as the core 2 β-1,6-GlcNAc transferase, and Mnn9 protein, as the protein localized in the Golgi apparatus. In Examples of the present invention, core 2 GnT1(C2GnT1) transferase was selected as the core 2 β-1,6-GlcNAc transferase, and Mnn9 protein, as the protein localized in the yeast Golgi apparatus. The nucleotide sequence and amino acid sequence of a gene encoding a protein in which the transmembrane region of Mnn9 is fused to FLAG antigen and the catalytic domain of C2GnT1 are shown in SEQ ID NOS: 80 and 81, respectively, in Sequence Listing.

### (7) A Gene Encoding Core 3 β-1,3-GlcNAc Transferase

Core 3 β-1,3-GlcNAc transferase has not been identified so far in yeast.

The transfer reaction of a sugar chain occurs mainly in the lumen of the Golgi apparatus. For expressing the above glycosyltransferase in the yeast Golgi apparatus, it is more preferable to add thereto the transmembrane region of a protein localized in the yeast Golgi apparatus. As the protein localized in the yeast Golgi apparatus there may be used a conventional known of well-known one; examples thereof include, but not limited to, the budding yeast glycosyltransferases Och1 protein (GenBank Accession No. D11095), Mnn9 protein (GenBank Accession No. L23752) and Kre2 protein (GenBank Accession No. X62647).

For allowing core 3 β-1,3-GlcNAc transferase to function successfully in the lumen of the yeast Golgi apparatus, a gene design is made, for example, so that the gene therefor comprises a nucleotide sequence encoding a protein in which the transmembrane region of a protein localized in the yeast Golgi apparatus is fused to a region comprising the catalytic domain of the core 3 β-1,3-GlcNAc transferase. By way of example, human β3-GnT-6 may be selected as the core 3 β-1,3-GlcNAc transferase, and Mnn9 protein, as the protein localized in the Golgi apparatus. In Examples of the present invention, human β3-GnT-6 (β3GnT6) was selected as the core 3 β-1,3-GlcNAc transferase, and Mnn9 protein, as the protein localized in the yeast Golgi apparatus. The nucleotide sequence and amino acid sequence of a gene encoding a protein in which the transmembrane region of Mnn9 is fused to β3GnT6 are shown in SEQ ID NOS: 80 and 81, respectively, in Sequence Listing.

### 3. Yeast

As the yeast employed as a host there may be used a conventional known or well-known one. Examples of the yeast include the budding yeast Saccharomyces cerevisiae and a methanol-utilizing yeast. Particularly, the methanol-utilizing yeast is preferable in that the use thereof enables: 1) the enhanced production of the mucin-type glycoprotein because the yeast has a strong promoter induced by methanol; 2) the selective inducible production of the mucin-type glycoprotein when the glycoprotein is harmful to the yeast cell; and 3) the suppression of addition of an O-type sugar chain characteristic of yeast to protein when a system is used which can inhibit the formation of the O-type sugar chain during addition of methanol. Examples of the methanol-utilizing yeast include Pichia methanolica and Hansenula polymorpha (Pichia angusta) in addition to Pichia pastoris and Ogataea minuta. However, yeasts other than the methanol-utilizing yeast can be also used; examples of such yeasts include Shizosaccharomyces pombe, Kluyveromyces lactis, and Yarrowia lipolytica in addition to Saccharomyces cerevisiae.

When a foreign protein is produced by a genetic recombination method, the desired product may be degraded by a host-derived protease to decrease the production thereof, to generate heterogenous proteins, or to make difficult the purification of the desired protein because of the contamination of protein degradation products. To avoid such problems, there are discussed culture methods as comprising the inhibition of protease activity degrading a desired protein, such as the inhibition of protease action by properly adjusting the pH of a medium for culturing a recombinant. However, these methods affect the growth of host yeast.

In contrast, protease-deficient yeast strains are excellent in that they can efficiently produce foreign proteins highly sensitive to proteases such as an antibody by suppressing the degradation thereof.

By way of example, it has been described that protease-deficient strains of Saccharomyces cerevisiae, Pichia pastoris and Candida boidinii, in which proteinases A and B were inactivated, were used to increase the intra- and extracellular protein production (JP Patent Publication (Kohyo) No. 6-506117 (1994), Weis, H. m. et al., FEBS Lett., 377: 451 (1995); Inoue, K. et al., Plant Cell Physiol., 38(3): 366 (1997); and JP Patent Publication (Kokai) No. 2000-078978). It has been also described that a Saccharomyces cerevisiae strain in which Yapsin 1 was inactivated was used as a protease-deficient strain to increase the intra- and extracellular protein production (M. Egel-Mitani et al., Enzyme and Microbial Technology, 26: 671 (2000); Bourbonniais, Y. et al., Protein Expr. Purif., 20: 485 (2000)).

A disruptant yeast strain in which sugar chain synthesis-associated genes and the like specific to yeast as a host are disrupted is also preferable in that the strain has enhanced expression of an introduced gene and efficiently synthesizes a mucin-type sugar chain. Examples of the disruptant strain can include a pmt disruptant (Gentzsch and Tanner, EMBO J., 15: 5752-5759 (1996)), a kre 2 disruptant (GenBank Accession No. X62647), and a ktr disruptant (Lussier et al., Biochim. Biophys. Acta, 1426: 323-334 (1999)).

The disruption of a target gene can be carried out according to a method as disclosed, for example, in Rothstein, Methods Enzymol., 101: 202-211 (1983). Specifically, a target gene obtained by the above method may be split or partially deleted, into which a suitable selectable marker gene is then inserted to prepare a DNA structure in which the selectable marker is sandwiched between the upstream and downstream portions of the target gene, followed by introducing the structure into yeast cells. By the above operation, two recombinations occur between both ends of the introduced fragment (a DNA structure having the selectable marker sandwiched) and homologous portions thereto in a target gene on the chromosome to replace the target gene on the chromosome with the introduced fragment. As the selectable marker here used for gene disruption there is employed an auxotrophic or drug-resistance marker as described below. In this case, the disruption of one gene typically requires one selectable marker; however, when URA3 gene is used, ura3 trait can be efficiently reproduced and so it is often used for this purpose.

The disruption method is specifically as follows. A plasmid is constructed which has a URA3 gene having iterative structures across the structural gene, and the gene cassette is excised with a restriction enzyme and inserted into a target gene on a plasmid to construct a disrupted allele. Using this plasmid, a target gene on the chromosome is replaced with the allele to provide a gene-disrupted strain. Because the URA3 gene inserted into the chromosome has iterative structures thereacross, the gene is deleted from the chromosome due to homologous recombination between the repetitive sequences. The deletion strain can be selected using 5-fluoroorotic acid (5-FOA). The Ura3 variant is resistant to 5-FOA (Boeke et al., mol. Gen. Genet., 197: 345-346 (1984); Boeke et al., Methods Enzymol., 154: 165-174 (1987)), and the cell strain having Ura⁺ phenotype cannot grow in a 5-FOA medium. Consequently, separating a strain having the resistant trait using the 5-FOA-added medium makes again possible the operation employing the URA3 gene marker. Thus, in the “artificial disruptant” obtained by artificial gene disruption using the procedure, the auxotrophic mutation trait of the original yeast strain is not impaired by the gene disruption process. In this respect, there may be also used a “spontaneous variant (mutant)” in which the gene disruption has been spontaneously generated in the absence of artificial disruption, in which case the number of auxotrophic mutation traits is maintained.

In addition to the use of the foregoing disruptant, the synthesis of a mucin-type sugar chain specific to yeast may be also suppressed by culturing the yeast under suitable conditions of suppressing sugar chain synthesis-associated genes and the like specific to the yeast. Methods for the synthesis suppression include, for example, culture in the presence of a compound inhibiting the activity of an O-mannosylation enzyme. Examples of the O-mannosylation enzyme include PMT. Examples of a compound inhibiting the PMT activity include a rhodamine-3-acetic acid derivative (Bioorganic & Medicinal Chemistry Letters, 14: 3975-3978 (2004)).

A methanol-utilizing yeast such as Ogataea minuta, which can grow by use of methanol as a carbon source, has a strong promoter for a metabolism-associated gene used during methanol metabolism, and is excellent in that the use of the promoter enables the abundant expression of protein. Because the yeast is viable even under culture conditions of relatively low temperature, it can be expected that the expression of protein at low temperature leads to no action of protease and thereby the suppression of degradation thereof.

### 4. A Transformed Yeast

### Yeast Transformant

The above-described mucin-type glycoprotein synthesis-associated gene is introduced into a yeast as a host by use of a suitable vector to prepare a yeast transformant of the present invention.

The vector for introducing the mucin-type glycoprotein synthesis-associated gene to express the gene can be obtained by linking (inserting) the gene to a known vector such as plasmid. The vector used is not particularly limited provided that it can be replicated in yeast; examples thereof include plasmid DNA and phage DNA.

Examples of the plasmid DNA include E. coli-derived plasmids (e.g., pBR322, pBR325, pUC18, pUC19, pTrcHis, and pBlueBacHis), bacillus subtilis-derived plasmids (e.g., pUB110 and pTP5), and yeast-derived plasmids (e.g., YEp13, YEp24, YCp50, and pYE52); examples of the phage DNA include λ-phage.

The insertion of the mucin-type glycoprotein synthesis-associated gene into the above vector uses a method involving first cleaving the purified DNA with a suitable restriction enzyme and then inserting the cleaved DNA into an appropriate restriction enzyme site or multicloning site of vector DNA for linkage to the vector. The gene introduced can be prepared, for example, by using as a template a genome gene or cDNA library containing the gene region to design such sense and antisense primers as to provide a desired gene fragment, followed by gene amplification such as PCR. The amplified gene fragments may be, if necessary, ligated with a DNA ligase or the like to prepare a desired gene fragment. When restriction enzyme sites are properly added to both ends of the prepared gene, an appropriate restriction enzyme and DNA ligase can be used to construct a desired plasmid.

For expressing a foreign gene in a host, it is necessary to place a suitable promoter before the structural gene. The promoter used is not particularly limited and may be any promoter known to function in yeast as a host. Examples thereof include the GAL1 promoter, GAL10 promoter, heat-shock protein promoter, MFα1 promoter, PHO5 promoter, and AOX promoter. Cis elements such as an enhancer, a splicing signal, a poly(A) addition signal, a ribosome binding sequence (SD sequences), a terminator sequence, and the like may be also placed in the vector, if necessary.

Methods for introducing the vector into yeast include an electroporation method (Becker, D. M. et al., Methods. Enzymol., 194: 180 (1990)), a spheroplast method (Hinnen, A. et al., Proc Natl. Acad. Sci. USA, 75: 1929 (1978)), and a lithium acetate method (Itoh, H., J. Bacteriol., 153: 163 (1983)).

The DNA once introduced into a plasmid can be easily amplified, isolated, and purified by use of E. coli. The DNA sequencing or the like of the gene can be performed, for example, by a dideoxy method, or can be conveniently carried out using a commercial sequencing kit or the like.

### 5. A Method for Producing a Mucin-Type Glycoprotein

The present invention also provides a method for producing a mucin-type glycoprotein, using the yeast transformant of the invention. Specifically, the yeast transformant of the present invention can be cultured in a suitable medium, followed by collecting a mucin-type glycoprotein from the culture. Here, “culture” refers to any of a culture supernatant, cultured cells or cell bodies, and a crushed product of the cells or cell bodies. The method for culturing the transformant of the present invention in a medium is performed according to a conventional method used for yeast culture.

The medium used may be any of a natural medium and a synthetic medium provided that it is a medium containing carbon and nitrogen sources, inorganic salts, and the like utilizable by yeast, the use of which enables the yeast transformant to be efficiently cultured.

The carbon source used is a carbohydrate such as glucose, fructose, sucrose, and starch, an organic acid such as acetic acid and propionic acid, or an alcohol such as ethanol and propanol. Examples of the nitrogen source used include peptone, meat extract, and corn steep liquor as well as ammonia, inorganic or organic acid ammonium salts such as ammonium chloride, ammonium sulfate, ammonium acetate, and ammonium phosphate and other nitrogen-containing compounds.

Examples of the inorganic salt used include potassium dihydrogen phosphate, di-potassium monohydrogen phosphate, magnesium phosphate, magnesium sulfate, sodium chloride, ferrous sulfate, manganese sulfate, copper sulfate, and calcium carbonate. The culture is typically carried out at about 30 to 37° C. for the order of 6 hours to 3 days under aerobic conditions such as shake culture and aerated and agitated culture. The pH is kept at a value of the order of 4.5 to 6.0 during the period of culture. The pH is adjusted using an inorganic or organic acid, an alkali solution, or the like.

An antibiotic such as ampicillin and tetracycline may be added as needed to the medium during the culture. When a yeast transformed with an expression vector using an inducible promoter as a promoter is cultured, an inducer may be added to the medium, if necessary. By way of example, galactose or the like may be added to the medium when a yeast transformed with an expression vector using the GAL promoter is cultured; copper sulfate or the like, when a yeast transformed with an expression vector using the CUP1 promoter is cultured; and methanol, when a yeast transformed with an expression vector using a methanol-inducible promoter (AOX, MOX, DAS, or the like) is cultured.

The culture is typically performed at about 20 to 30° C. for the order of 1 to 20 days. When the mucin-type glycoprotein is produced in cell bodies, the protein is extracted by crushing the cell bodies after culture. When the protein of the present invention is secreted outside cell bodies, the culture solution is directly used or the cell bodies are removed by centrifugation. Subsequently, the mucin-type glycoprotein can be isolated and purified from the culture by using a common biochemical method such as ammonium sulfate precipitation, gel chromatography, ion exchange chromatography, and affinity chromatography, alone or in a proper combination thereof.

### 6. Use of the Yeast-Produced Mucin-Type Glycoprotein

Mucin-type glycoproteins generated in mammals including human can be inexpensively and efficiently produced on a large scale when genes encoding various useful glycoproteins are each introduced into the yeast transformant of the present invention. The resultant human mucin-type glycoprotein is used as a useful protein e.g., in the form of a human antigen employed for cancer immunotherapy or an antibody drug having a human sugar chain.

Examples of the gene introduced include genes encoding useful proteins having mucin-type sugar chains, such as CD34, CD43, and various antigens and antibodies. Particularly, for proteins for medical use such as an antigen and an antibody, the technique of the present invention has extremely high usefulness as will be described below. In this respect, for the purpose of the present invention, the antigen and antibody include not only intact antigen and antibody molecules but also functional fragments thereof.

Useful expression systems for producing foreign proteins as described above can be prepared by various methods. The protein expression vector comprises at least a promoter region, a DNA encoding a protein, and a transcription terminator region which are oriented in the direction of the reading frame for transcription. These DNAs are arranged so that a DNA encoding a desired glycoprotein is transcribed to RNA. The vector used is exemplified by a vector for budding yeast such as YPp, YIp and YCp vectors, having a promoter such as TDH3, GAL1, and CUP1, or an E. coli-methanol yeast shuttle vector containing an auxotrophic marker gene, an AOX promoter, and the like.

In the vector, a DNA encoding a secretion signal sequence functioning in the yeast cell may be added to the foreign structural gene. This enables secretory production and permits a desired glycoprotein to be easily isolated and purified. Examples of the secretion signal sequence include the secretion signal sequences of Saccharomyces cerevisiae-derived α-mating factor (αMF), Saccharomyces cerevisiae-derived invertase (SUC2), human α-galactosidase and a human antibody light chain. A molecular chaperonin or the like may be further introduced to enhance secretion efficiency.

The expression vector is a chromosome incorporation-type vector, and the incorporation thereof into the chromosome results in the introduction of an intended gene. For an auxotrophic marker-type vector, the vector is cleaved in the region of the marker gene by use of a restriction enzyme and made into a single strand. Then, the transformation is performed therewith to typically introduce the marker into the region of the allele on the chromosome. For a drug-resistance marker-type vector, because the allele of the marker is absent, the vector is cleaved in the portion of the expression promoter or terminator by use of a restriction enzyme and made into a single strand. Then, the transformation is performed therewith to typically introduce the marker into the portion on the chromosome. The gene once introduced is stably maintained because it is present on the chromosome.

### EXAMPLES

### Example 1

### Production of Mucin-Type Glycopeptide-Producing Yeast Strains Using S. cerevisiae

GalE gene derived from Bacillus subtilis is present on the genome of Bacillus subtilis, and its cDNA sequence has been deposited in the public database GenBank under the Accession No. P55180. First, the full-length cDNA of the GalE gene was amplified by PCR using the genome DNA of the Bacillus subtilis strain 168 as a template and primer A (SEQ ID NO: 11) and primer B (SEQ ID NO: 12) as primers.

| | |
|---|---|
| SEQ ID NO: 11: | GGAATTCATGTTAATTAACGCAATACTTGTTACT |
| SEQ ID NO: 12: | GCTCTAGATTATTCCGCACTCTTATA |

The resultant PCR product was cleaved at the EcoRI and XbaI sites and incorporated into the EcoRI/XbaI site of plasmid pBluescript II(SK−) (from Stratagene), a plasmid for cloning in E. coli, to construct plasmid pBluescript II(SK−)-GalE. Then, a PaCI site-containing DNA sequence for expressing the myc antigen was prepared by PCR using primer C (SEQ ID NO: 13) and primer D (SEQ ID NO: 14) and cleaved at the PacI site. Subsequently, pBluescript II(SK−)-GalE was cleaved at the PacI site, and the DNA sequence for expressing the myc antigen was inserted into the 5′ region of the GalE to construct pBluescriptII (SK−)-myc/GalE.

| | |
|---|---|
| SEQ ID NO: 13: | CCTTAATTAActctgaacaaaaactcatctcagaagaggatctgaatggatctgaacaaaaac |
| SEQ ID NO: 14: | CCTTAATTAAcgatccattcagatcctcttctgagatgagtttttgttcagatccattcaga |

The GalE gene containing the myc antigen gene was excised from the plasmid at the EcoRI and XbaI sites and inserted into plasmid YEp352GAPII (Abe et al, Glycobiology, 13: 87-95 (2003)), a plasmid for expression in yeast, at the EcoRI and XbaI sites to construct YEp352GAPII-myc/GalE. A region containing the yeast GAPDH promoter and terminator was further excised with BamHI from the plasmid and inserted into the BamHI site of plasmid pUC19, a plasmid for cloning in E. coli, to construct pUC19-GAPDH/myc/GalE. A region containing the GAPDH promoter and terminator was excised from the constructed plasmid at the HindIII and SacI sites and inserted into the HindIII and SacI sites of the integration vector pRS306 (from Clontech) to prepare pRS306-myc/GalE. The plasmid pRS306-myc/GalE was cleaved with EcoRV, followed by transforming the S. cerevisiae strain W303-1A (Kainuma et al., Glycobiology, 9: 133-141 (1999)) therewith. The transformation was carried out using a lithium acetate method. The transformed strain was inoculated into an SD-Ura (2% glucose, 0.17% Yeast Nitrogen Base w/o amino acids (from Difco), nucleobases other than uracil, and an amino acid mixture (20 to 400 mg/L)) medium, and cultured at 30° C. for 2 days to provide a transformant. The transformant was scraped from the plate, confirmed to have been incorporated onto the chromosome by a simplified PCR method involving the suspension thereof in a PCR reaction solution, and called strain KAY-1.

The UGT2 gene is located in the human chromosome 10, and its cDNA sequence has been deposited in the public database GenBank under the Accession No. D87969. First, the full-length UGT2 gene was amplified by PCR using primer E (SEQ ID NO: 15) and primer F (SEQ ID NO: 16).

| | |
|---|---|
| SEQ ID NO: 15: | GGAATTCATGGCAGCGGTTGGGGC |
| SEQ ID NO: 16: | GCTCTAGACTAGTTAATTAAGGAACCCTTCACCTT |

A gene providing a template was a gift from Dr. Nobuhiro Ishida, Tokyo Metropolitan Institute of Medical Science. The PCR product was cleaved at the EcoRI and XbaI sites and incorporated into the EcoRI/XbaI site of plasmid pBluescript II(SK−), a plasmid for cloning in E. coli, to construct plasmid pBluescript II(SK−)-UGT2. Then, a PacI site-containing DNA sequence for expressing the HA antigen was prepared by PCR using primer G (SEQ ID NO: 17) and primer H (SEQ ID NO: 18) and cleaved at the PacI site. Subsequently, pBluescript II(SK−)-UGT2 was cleaved at the PacI site, and the DNA sequence for expressing the HA antigen was inserted into the 3′ region of UGT2 to construct pBluescriptII (SK−)-HA/UGT2.

| | |
|---|---|
| SEQ ID NO: 17: | CCTTAATTAActacccatacgatgttcc |
| SEQ ID NO: 18: | CCTTAATTAATCgtcgacaccaagccgaat |

The UGT2 gene containing the HA antigen gene was excised from the plasmid at the EcoRI and XbaI sites and inserted into plasmid YEp352GAPII, a plasmid for expression in yeast, at the EcoRI and XbaI sites to construct YEp352GAPII-HAUGT2. A region containing the yeast GAPDH promoter and terminator was further excised with BamHI from the plasmid and inserted into the BamHI site of plasmid pUC19, a plasmid for cloning in E. coli, to construct pUC19-GAPDH/HA/UGT2. A region containing the GAPDH promoter and terminator was excised from the constructed plasmid at the HindIII and SacI sites and inserted into the HindIII and SacI sites of the integration vector pRS305 to prepare pRS305-HA/UGT2. The plasmid pRS305-HA/UGT2 was cleaved with EcoRV, followed by transforming the strain KAY-1 therewith. The transformation was carried out using a lithium acetate method. The transformed strain was inoculated into an SD-Leu (2% glucose, 0.17% Yeast Nitrogen Base w/o amino acids (from Difco), nucleobases other than leucine, and an amino acid mixture (20 to 400 mg/L)) medium, and cultured at 30° C. for 2 days to provide a transformant. The transformant was scraped from the plate, confirmed to have been incorporated onto the chromosome by a simplified PCR method involving the suspension thereof in a PCR reaction solution, and called strain KAY-2.

The ppGalNAc-T1 gene is located in the human chromosome 18, and its cDNA sequence has been deposited in the public database GenBank under the Accession No. X85018. First, the catalytic domain of the ppGalNAc-T1 gene was amplified by PCR using primer I (SEQ ID NO: 19) and primer J (SEQ ID NO: 20).

| | |
|---|---|
| SEQ ID NO: 19: | CGAGCTCATGTCACTTTCTCTTGTATC |
| SEQ ID NO: 20: | GCGTGTCGACTCAGAATATTTCTGGCAGG |

As a template was used a plasmid into which there was incorporated the ppGalNAc-T1 gene, which had already been cloned in the NEDO “Tosa Idenshi Raiburarino Kochiku (Construction of a glycogene library)” project (Industrial and scientific technology development project for FY 2003: Research Report on Construction of Libraries of Human Genes Participating in Glycosylation Project (Biotechnology Basic Research Program for Health Maintenance and Improvement) subcontracted by New Energy and Industrial Technology Development Organization, from National Institute of Advanced Industrial Science and Technology and Research Association for Biotechnology (2004)) (Shimma and Jigami, Glycoconj. J., 21: 75-78 (2004)). The PCR product was cleaved at the SpeI and BamHI sites and incorporated into the SpeI/BamHI site of plasmid pUC19 (from Takara Bio Inc.) for cloning in E. coli, into which there had already been inserted the gene sequence of the transmembrane domain of MNN9, a subunit of mannosyltransferase in the Golgi apparatus of S. cerevisiae, to construct plasmid pUC19-MNN9/ppGalNAc-T1. Then, a SpeI site-containing DNA sequence for expressing the FLAG antigen was prepared by PCR using primer K (SEQ ID NO: 21) and primer L (SEQ ID NO: 22) and cleaved at the SpeI site. Subsequently, pUC19-MNN9/ppGalNAc-T1 was cleaved at the SpeI site, and the DNA sequence for expressing the FLAG antigen was inserted into the 5′ region of catalytic domain of ppGalNAc-T1 to construct pUC19-MNN9/FLAG/ppGalNAc-T1.

| | |
|---|---|
| SEQ ID NO: 21: | GACTAGTGACTACAAAGACCATG |
| SEQ ID NO: 22: | GACTAGTCTTGTCATCGTCATC |

The ppGalNAc-T1 gene containing the FLAG antigen gene at the BamHI site was cleaved at the SalI and SacI sites from the plasmid and inserted into plasmid YEp352GAPII, a plasmid for expression in yeast, at the SalI/SacI site to construct YEp352GAPII-MNN9/FLAG/ppGalNAc-T1. A region containing the yeast GAPDH promoter and terminator was further excised with BamHI from the plasmid, blunted at both ends, and inserted into the SmaI/HincII site of the integration vector pRS304 to prepare pRS304-MNN9/FLAG/ppGalNAc-T1. The plasmid pRS304-MNN9/FLAG/ppGalNAc-T1 was cleaved with EcoRV, followed by transforming the strain KAY-2 therewith. The transformation was carried out using a lithium acetate method. The transformed strain was inoculated into an SD-Trp (2% glucose, 0.17% Yeast Nitrogen Base w/o amino acids (from Difco), nucleobases other than tryptophan, and an amino acid mixture (20 to 400 mg/L)) medium, and cultured at 30° C. for 2 days to provide a transformant. The transformant was scraped from the plate, confirmed to have been incorporated onto the chromosome by a simplified PCR method involving the suspension thereof in a PCR reaction solution, and called strain KAY-3.

### Example 2

### Introduction of the MUC1a Peptide Gene into Mucin-Type Glycopeptide-Producing S. cerevisiae Strains and the Production of the Peptide

The MUC1 gene is located in the human chromosome 1, and the MUC1a gene has a nucleotide sequence (nucleotide 253 to 288 in SEQ ID NO: 7) derived from the internal repeat sequence of the MUC1 gene. The MUC1a gene was synthesized by PCR using primer M (SEQ ID NO: 23) and primer N (SEQ ID NO: 24).

SEQ ID NO: 23:
CGGGATCCGGTCTAGATAAAAGAGCTCATGGTGTTACTTCTGCT
CCAGACACTAG
SEQ ID NO: 24:
ACTTCTGCTCCAGACACTAGACATCACCATCACCATCACTAATC
TAGAGGATCCCG

Here, an amino acid sequence (SEQ ID NO: 8) was designed so that a His6 tag for purification was fused to the 3′ region of the MUC1a gene. The PCR product was cleaved with XbaI and incorporated into the XbaI site of plasmid YEp352GAPII-α-factor (Abe et al, Glycobiology, 13: 87-95 (2003)), a plasmid for expression in yeast, containing the α-factor sequence as a signal sequence for external secretion, to prepare YEp352GAPII-alfaMUC1aHis. A region containing the GAPDH promoter and terminator was excised from the plasmid at the BamHI site and inserted into the integration vector pRS303 at the BamHI site to construct pRS303-alfaMUC1aHis. The plasmid pRS303-alfaMUC1aHis was cleaved with NheI, followed by transforming the strain KAY-3 therewith. The transformation was carried out using a lithium acetate method. The transformed strain was inoculated into an SD-His (2% glucose, 0.17% Yeast Nitrogen Base w/o amino acids (from Difco), nucleobases other than histidine, and an amino acid mixture (20 to 400 mg/L)) medium, and cultured at 30° C. for 2 days to provide a transformant. The transformant was scraped from the plate, confirmed to have been incorporated onto the chromosome by a simplified PCR method involving the suspension thereof in a PCR reaction solution, and called strain KAY-4.

Then, strains KAY-5 to KAY-9 were prepared as strains used as a control or in Examples to be described, by the following procedure.

The strain KAY-5 was prepared by cleaving the above pRS303-alfaMUC1aHis with NheI and then transforming the strain KAY-2 therewith. The transformation was carried out using a lithium acetate method. The transformed strain was inoculated into an SD-His (2% glucose, 0.17% Yeast Nitrogen Base w/o amino acids (from Difco), nucleobases other than histidine, and an amino acid mixture (20 to 400 mg/L)) medium, and cultured at 30° C. for 2 days to provide a transformant. The transformant was scraped from the plate and confirmed to have been incorporated onto the chromosome by a simplified PCR method involving the suspension thereof in a PCR reaction solution to make strain KAY-5.

The strain KAY-6 was prepared by cleaving the above pRS304-MNN9/FLAG/ppGalNAc-T1 with EcoRV and then transforming the strain KAY-1 therewith. The transformation was carried out using a lithium acetate method. The transformed strain was inoculated into an SD-Trp (2% glucose, 0.17% Yeast Nitrogen Base w/o amino acids (from Difco), nucleobases other than tryptophan, and an amino acid mixture (20 to 400 mg/L)) medium, and cultured at 30° C. for 2 days to provide a transformant. The transformant was scraped from the plate and confirmed to have been incorporated onto the chromosome by a simplified PCR method involving the suspension thereof in a PCR reaction solution to make strain KAY-6.

The strain KAY-7 was prepared by cleaving the above pRS303-alfaMUC1aHis with NheI and then transforming the strain KAY-6 therewith. The transformation was carried out using a lithium acetate method. The transformed strain was inoculated into an SD-His (2% glucose, 0.17% Yeast Nitrogen Base w/o amino acids (from Difco), nucleobases other than histidine, and an amino acid mixture (20 to 400 mg/L)) medium, and cultured at 30° C. for 2 days to provide a transformant. The transformant was scraped from the plate and confirmed to have been incorporated onto the chromosome by a simplified PCR method involving the suspension thereof in a PCR reaction solution to make strain KAY-7.

The strain KAY-8 was prepared by cleaving the above pRS304-MNN9/FLAG/ppGalNAc-T1 with EcoRV and then transforming the strain W303-1A therewith. The transformation was carried out using a lithium acetate method. The transformed strain was inoculated into an SD-Trp (2% glucose, 0.17% Yeast Nitrogen Base w/o amino acids (from Difco), nucleobases other than tryptophan, and an amino acid mixture (20 to 400 mg/L)) medium, and cultured at 30° C. for 2 days to provide a transformant. The transformant was scraped from the plate and confirmed to have been incorporated onto the chromosome by a simplified PCR method involving the suspension thereof in a PCR reaction solution to make strain KAY-8.

The strain KAY-9 was prepared by cleaving the above pRS303-alfaMUC1aHis with NheI and then transforming the strain KAY-8 therewith. The transformation was carried out using a lithium acetate method. The transformed strain was inoculated into an SD-His (2% glucose, 0.17% Yeast Nitrogen Base w/o amino acids (from Difco), nucleobases other than histidine, and an amino acid mixture (20 to 400 mg/L)) medium, and cultured at 30° C. for 2 days to provide a transformant. The transformant was scraped from the plate and confirmed to have been incorporated onto the chromosome by a simplified PCR method involving the suspension thereof in a PCR reaction solution to make strain KAY-9.

### Example 3

### Confirmation of Generation of UDP-Gal and UDP-GalNAc in a Mucin-Type Glycopeptide-Producing S. cerevisiae Strain

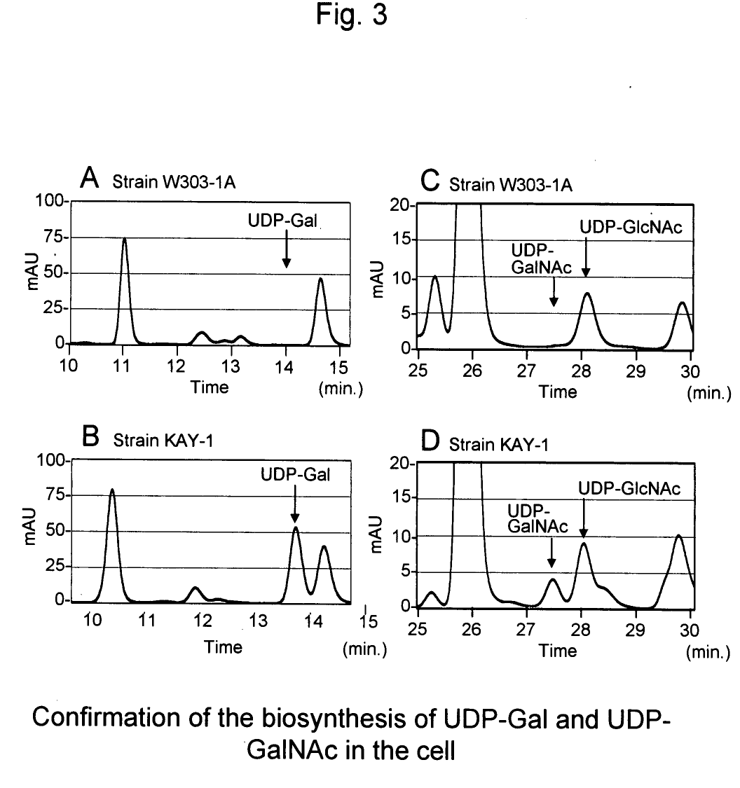

To confirm the generation of UDP-Gal and UDP-GalNAc in the S. cerevisiae strains KAY-1 and W303-1A, the analysis of the UDP-sugars was carried out by reversed-phase HPLC. The column used was Cosmosil C18 (4.6×250 mm: from Nacalai Tesque), and the solvent used was a 100 mM potassium dihydrogen phosphate buffer (pH 6.2) containing 2 mM tetrabutylammonium phosphate. The column was equilibrated by running the solvent in advance at a flow rate of 0.6 ml/min., into which a sample was then injected for analysis. The detection was carried out using a UV detector (detection wavelength: 262 nm). The results are shown in FIG. 3. A peak not observed for the strain W303-1A as a control, i.e. a peak corresponding to UDP-GalNAc around 27.5 minutes was seen for the strain KAY-1. A peak corresponding to UDP-Gal was also seen around 14 minutes for the strain KAY-1 while a peak corresponding thereto was not observed for the strain W303-1A as control. This suggests that the introduced Bacillus subtilis-derived GalE gene functioned normally in the yeast cell to convert UDP-GlcNAc to UDP-GalNAc as well as UDP-Glc to UDP-Gal. These results confirmed that UDP-GalNAc and UDP-Gal were generated in the yeast cell.

### Example 4

### Confirmation of Expression of Each Gene in a Mucin-Type Glycopeptide-Producing S. cerevisiae Strain

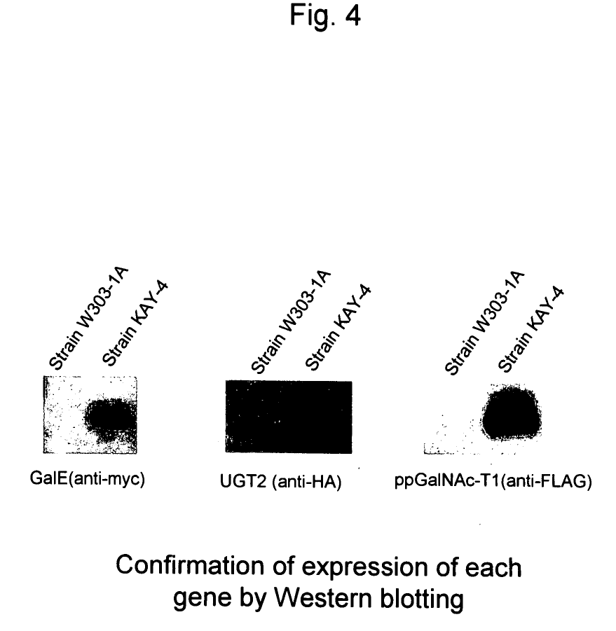

The S. cerevisiae strains KAY-4 and W303-1A were each cultured at 30° C. for 24 hours in 5 ml of a YPAD medium (2% polypeptone, 1% yeast extract, 2% glucose, adenine (40 mg/L)), followed by recovering the cell bodies by centrifugation. The cell bodies were crushed with glass beads, and a surfactant was added to the crush solution to solubilize insoluble protein, followed by centrifugation to provide a supernatant. The resultant supernatant was used as a solution of a crude enzyme, which was then denatured with SDS sample buffer, followed by western blot analysis using an ordinary method. The western blot analysis was performed using the mouse anti-HA antibody 16B12, mouse anti-FLAG antibody M2 or mouse anti-myc antibody 4A6 as a primary antibody and an anti-mouse Ig antibody-horseradish peroxidase complex as a secondary antibody; the detection was carried out on a chemiluminescence detector (Fujifilm Corporation) using a ECL-plus system (from Amersham). The results are shown in FIG. 4.

According to FIG. 4, no signal was observed for the strain W303-1A as a control strain, while for the transformant strain the respective signals were confirmed at the positions of molecular weights corresponding to a UGT2 gene product with the anti-HA antibody, a ppGalNAc-T1 gene product with the anti-FLAG antibody and a GalE gene product with the anti-myc antibody.

### Example 5

### Expression and Analysis of the MUC1a Peptide Gene in a Mucin-Type Glycopeptide-Producing S. cerevisiae Strain

The S. cerevisiae strain KAY-4 was cultured at 30° C. for 48 hours in 100 ml of a YPAD medium (2% polypeptone, 1% yeast extract, 2% glucose, adenine (40 mg/L)), followed by centrifugation to provide a culture supernatant. For a control, the strain KAY-5 was cultured in the same manner as the S. cerevisiae strain KAY-4 to provide a culture supernatant. The culture supernatant was adjusted to a pH of 7.0, and TALON metal affinity resins (from Clontech) were added thereto, which was then washed with a phosphate buffer (pH 7.4) containing 0.5 M NaCl, followed by elution using a phosphate buffer (pH 7.4) containing 0.5 M NaCl and 0.5 M imidazole. The resultant eluate was used as a purified MUC1a preparation.

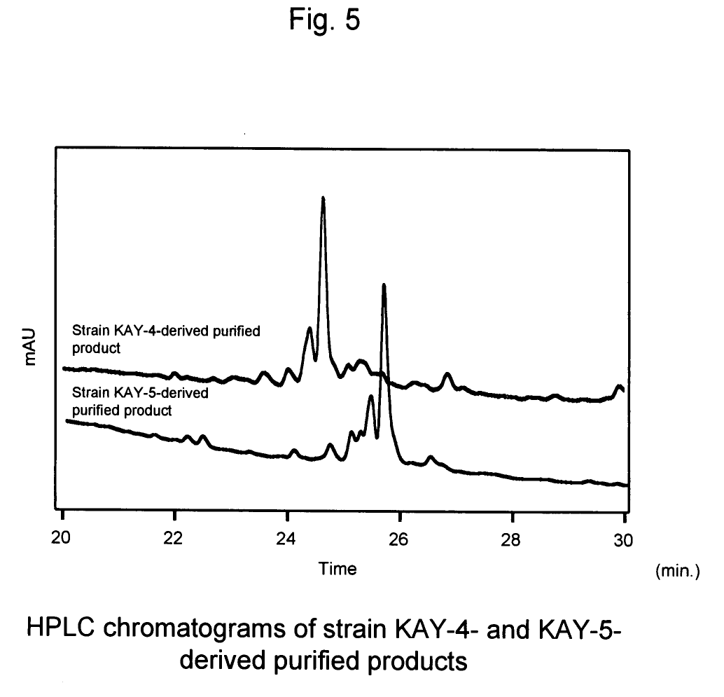

To quantitate the linking amount of GalNAc for the resultant MUC1a preparation, a purity test was performed by HPLC analysis using a reversed-phase column. The column used was Cosmosil 5C18-ARII (4.6×250 mm: from Nacalai Tesque), and the solvents used were 0.05% trifluoroacetic acid (solution A) and 0.05% trifluoroacetic acid-containing acetonitrile (solution B). The column was equilibrated by running the solvent A in advance at a concentration of 0.05% and a flow rate of 0.1 ml/min., and MUC1a was eluted by running the solvent A at a concentration of 0.05% and a flow rate of 0.1 ml/min. for 10 minutes after the injection of the sample and then linearly increasing the proportion of the solvent B to 20% over a period of 20 minutes. The detection was carried out using a UV detector (detection wavelength: 210 nm). The results are shown in FIG. 5. To test peaks on chromatogram, each peak was recovered.

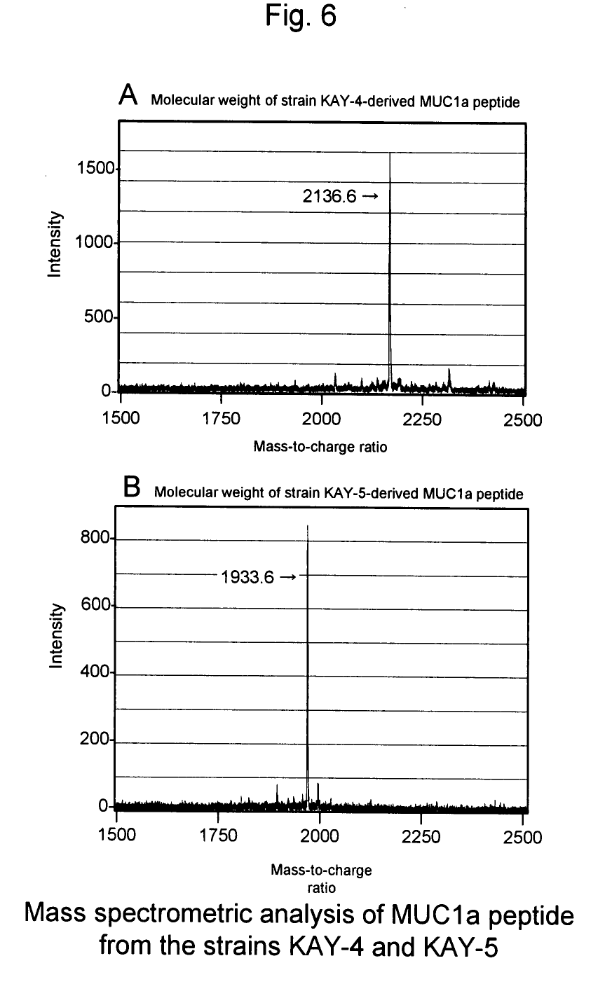

Each peak recovered from HPLC was made into a freeze-dried product and then subjected to molecular weight measurement using MALDI TOF-MS. The results are shown in FIG. 6. Peaks at elution times of about 25.5 min. and about 24.5 min. on HPLC were detected at positions of about 1934 and about 2137, respectively, corresponding to the molecular weights thereof. The molecular weight of the peak at about 25.5 min. agreed with the theoretical value of the MUC1a designed. In contrast, the molecular weight of the peak at about 24.5 min. was in agreement with the molecular weight of MUC1a to which the molecular weight of the N-acetylhexosamine moiety was added. These results demonstrated that one N-acetylhexosamine molecule was linked to MUC1a.

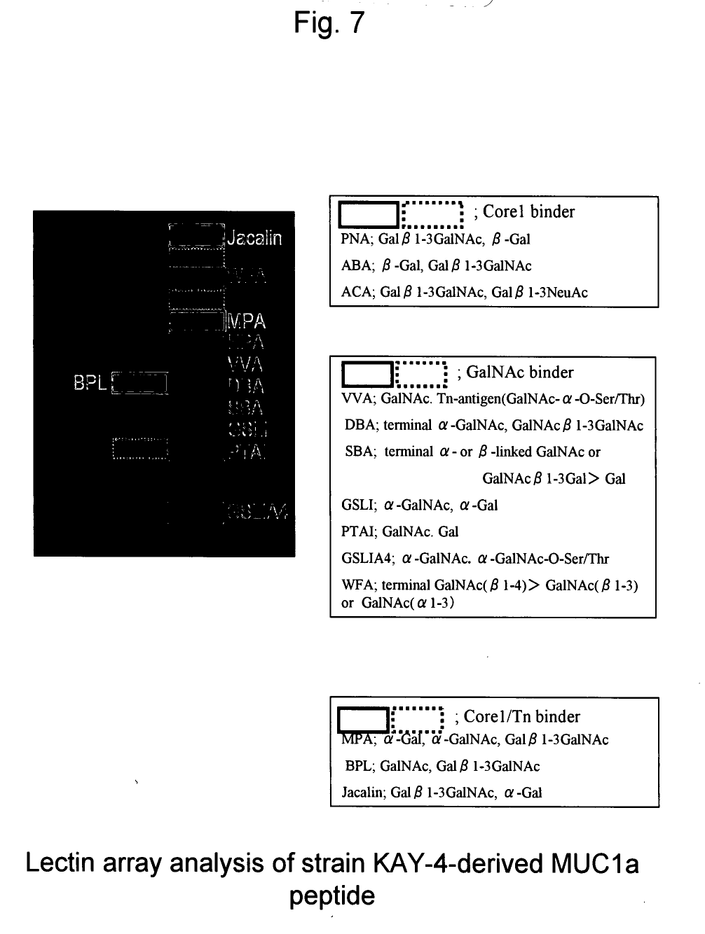

In order to examine whether the addition to MUC1a was GalNAc or not, analysis was performed using a lectin array (Kuno et al., Nat. Methods, 2: 851-856 (2005)). To 1 μg of the MUC1a peptide purified by HPLC were added 100 μg of Cy3-SE and 0.05% Tween-20-containing PBS, which was then reacted at room temperature for one hour to label the MUC1a with Cy3. The Cy3-labeled MUC1a peptide was subjected to lectin array analysis at a concentration of 10 μg/ml. The results are shown in FIG. 7. As a result of the lectin array analysis, the signals were observed on spots of the GalANc-specific lectins BPL (orchid tree-derived lectin), Jacalin (jackfruit-derived lectin), WFA (wisteria-derived lectin), MPA (osage orange-derived lectin), HPA (escargot-derived lectin), and VVA (hairy vetch-derived lectin). These results showed that GalNAc was added to the peptide. On the other hand, no signals were observed on these lectin spots for the KAY-5-derived peptide prepared as a control.

### Example 6

### Production of a Mucin-Type Glycopeptide-Producing S. Cerevisiae Strain Capable of Being Subjected to Gene Disruption

To perform gene disruption in mucin-type glycopeptide-producing strains by use of a hisG-URA3-hisG cassette, strains for gene disruption were prepared.

pRS305-HA/UGT2 was cleaved with EcoRV, followed by transforming the strain KAY-8 therewith. The transformation was carried out using a lithium acetate method. The transformed strain was inoculated into an SD-Leu (2% glucose, 0.17% Yeast Nitrogen Base w/o amino acids (from Difco), nucleobases other than leucine, and an amino acid mixture (20 to 400 mg/L)) medium, and cultured at 30° C. for 2 days to provide a transformant. The transformant was scraped from the plate, confirmed to have been incorporated onto the chromosome by a simplified PCR method involving the suspension thereof in a PCR reaction solution, and called strain KAY-10.

A region containing the yeast GAPDH promoter and terminator was then excised with BamHI from YEp352GAPII-myc/GalE and inserted into the integration vector pRS303 at the BamHI site to construct pRS303-myc/GalE. The plasmid pRS303-myc/GalE was cleaved with NheI, followed by transforming the strain KAY-10 therewith. The transformation was carried out using a lithium acetate method. The transformed strain was inoculated into an SD-His (2% glucose, 0.17% Yeast Nitrogen Base w/o amino acids (from Difco), nucleobases other than histidine, and an amino acid mixture (20 to 400 mg/L)) medium, and cultured at 30° C. for 2 days to provide a transformant. The transformant was scraped from the plate, confirmed to have been incorporated onto the chromosome by a simplified PCR method involving the suspension thereof in a PCR reaction solution, and called strain KAY-11.

### Example 7

### Introduction of the MUC1a Peptide Gene into a Mucin-Type Glycopeptide-Producing S. cerevisiae Strain Capable of being Subjected to Gene Disruption

A region containing the GAPDH promoter and terminator was excised from the YEp352GAPII-alfaMUC1aHis prepared in Example 2, at the HindIII and SacI sites and inserted into the integration vector pRS306 (from Clontech) at the HindIII and SacI sites to provide pRS306-alfaMUC1aHis. pRS306-alfaMUC1aHis was cleaved with EcoRV, followed by transforming the strain KAY-11 therewith. The transformation was carried out using a lithium acetate method. The transformed strain was inoculated into an SD-Ura (2% glucose, 0.17% Yeast Nitrogen Base w/o amino acids (from Difco), nucleobases other than uracil, and an amino acid mixture (20 to 400 mg/L)) medium, and cultured at 30° C. for 2 days to provide a transformant. The transformant was scraped from the plate, confirmed to have been incorporated onto the chromosome by a simplified PCR method involving the suspension thereof in a PCR reaction solution, and called strain KAY-12.

### Example 8

### Expression and Analysis of the MUC1a Peptide Gene in a Mucin-Type Glycopeptide-Producing S. cerevisiae Strain Capable of being Subjected to Gene Disruption

The S. cerevisiae strain KAY-11 was cultured at 30° C. for 48 hours in 100 ml of a YPAD medium (2% polypeptone, 1% yeast extract, 2% glucose, adenine (40 mg/L)), followed by centrifugation to provide a culture supernatant. For a control, the strain KAY-5 was cultured in the same manner as the S. cerevisiae strain KAY-12 to provide a culture supernatant. The culture supernatant was adjusted to a pH of 7.0, and TALON metal affinity resins (from Clontech) were added thereto, which was then washed with a phosphate buffer (pH 7.4) containing 0.5 M NaCl, followed by elution using a phosphate buffer (pH 7.4) containing 0.5 M NaCl and 0.5 M imidazole. The resultant eluate was used as a purified MUC1a preparation.

To quantitate the linking amount of GalNAc for the resultant MUC1a preparation, a purity test was performed by HPLC analysis using a reversed-phase column. The column used was Cosmosil 5C18-ARII (4.6×250 mm: from Nacalai Tesque), and the solvents used were 0.05% trifluoroacetic acid (solution A) and 0.05% trifluoroacetic acid-containing acetonitrile (solution B). The column was equilibrated by running the solvent A in advance at a concentration of 0.05% and a flow rate of 0.1 ml/min., and MUC1a was eluted by running the solvent A at a concentration of 0.05% and a flow rate of 0.1 ml/min. for 10 minutes after the injection of the sample and then linearly increasing the proportion of the solvent B to 20% over a period of 20 minutes. The detection was carried out using a UV detector (detection wavelength: 210 nm). Similar to the results in FIG. 5, a peak was eluted at the elution position of MUC1a to which N-acetylhexosamine was added, demonstrating that the mucin-type glycoprotein was produced even when the mucin-type glycoprotein synthesis-associated gene was introduced by exchanging the auxotrophic marker.

### Example 9

### Disruption of the PMT Gene in a Mucin-Type Glycopeptide-Producing S. cerevisiae Strain

Known O-mannosyltransferase (PMT) genes of the budding yeast S. cerevisiae strain are those of PMT1 to PMT6 (the function of PMT7 is not known). Accordingly, the strain KAY-11 is used as a parent strain to perform the disruption of the PMT1 to PMT6 genes.

A cassette (HUH) in which the Salmonella hisG gene is linked to both ends of the URA3 gene in direct repeat is cleaved with BglII and BamHI from pNK51, which has been already reported (Alani et al., Genetics, 116: 541-545 (1987)), and is inserted into the BamHI site of E. coli plasmid pSP73 (from Promega). This plasmid is designated as pSP73-HUH.

The cDNA sequences of PMT1 to PMT6 genes of the S. cerevisiae strain have been deposited in the public database GenBank under the Accession Nos. L19169, L05146, X83797, X83798, X95644, and Z72984, respectively. By an ordinary method, a genome DNA extracted from the S. cerevisiae strain W303-1A is used as a template to amplify the 5′ and 3′ ends of the genes using the following primers (SEQ ID NOS: 25 to 48).

| | | |
|---|---|---|
| SEQ ID NO: 25: | pmt1 5′ amplification primer 1-5S: | GCTTCAGCTGCTCGAGATCTT |
| | | TGCTTCAATTACGC |
| SEQ ID NO: 26: | pmt1 5′ amplification primer 1-5A: | GGAGACCGGCCTCGACCGTG |
| | | CTTGTAGCTGTTAGC |
| SEQ ID NO: 27: | pmt1 3′ amplification primer 1-3S: | GAGTCGACCTGCAGGAATTTC |
| | | CCAGTACTCTCCAC |
| SEQ ID NO: 28: | pmt1 3′ amplification primer 1-3A: | AGCAGCTGAAGCTTGATGTAC |
| | | TACGCTTCTGTTCC |
| SEQ ID NO: 29: | pmt2 5′ amplification primer 2-5S: | GCTTCAGCTGTCGAACTGTAC |
| | | ATACCGAAACGCC |
| SEQ ID NO: 30: | pmt2 5′ amplification primer 2-5A: | GGAGACCGGCCTCGAACATG |
| | | ATTGCTGGACCACGG |
| SEQ ID NO: 31: | pmt2 3′ amplification primer 2-3S: | GAGTCGACCTGCAGGGCCGAC |
| | | AAGCAAGAAGCATG |
| SEQ ID NO: 32: | pmt2 3′ amplification primer 2-3A: | AGCAGCTGAAGCTTGGGTGG |
| | | GCTAAAGGGTTCAAG |
| SEQ ID NO: 33: | pmt3 5′ amplification primer 3-5S: | GCTTCAGCTGCTCGAAGCTAA |
| | | AAGGAACCTGTCCC |
| SEQ ID NO: 34: | pmt3 5′ amplification primer 3-5A: | GGAGACCGGCCTCGACGCCA |
| | | CTCTGTACGGCATTG |
| SEQ ID NO: 35: | pmt3 3′ amplification primer 3-3S: | GAGTCGACCTGCAGGACTTGG |
| | | CATGGCTACCTACG |
| SEQ ID NO: 36: | pmt3 3′ amplification primer 3-3A: | AGCAGCTGAAGCTTGTCTGAT |
| | | CAATAAGCAAACCC |
| SEQ ID NO: 37: | pmt4 5′ amplification primer 4-5S: | GCTTCAGCTGCTCGAGCGATA |
| | | CACATCCTCTAACC |
| SEQ ID NO: 38: | pmt4 5′ amplification primer 4-5A: | GGAGACCGGCCTCGAAGACA |
| | | TCTGTACTTCTGTGG |
| SEQ ID NO: 39: | pmt4 3′ amplification primer 4-3S: | GAGTCGACCTGCAGGCGGAA |
| | | GTTGTTTCTAGAGAG |
| SEQ ID NO: 40: | pmt4 3′ amplification primer 4-3A: | AGCAGCTGAAGCTTGACATG |
| | | GCTACATCACTCCAG |
| SEQ ID NO: 41: | pmt5 5′ amplification primer 5-5S: | GCTTCAGCTGCTCGAGACTAT |
| | | ATTGTCCATCTGCG |
| SEQ ID NO: 42: | pmt5 5′ amplification primer 5-5A: | GGAGACCGGCCTCGAGATCC |
| | | ACCTTCAGCAAATGC |
| SEQ ID NO: 43: | pmt5 3′ amplification primer 5-3S: | GAGTCGACCTGCAGGAATCAT |
| | | GATCCATCCACCCG |
| SEQ ID NO: 44: | pmt5 3′ amplification primer 5-3A: | AGCAGCTGAAGCTTGTCCTTT |
| | | TACCGGTTCAATCC |
| SEQ ID NO: 45: | pmt6 5′ amplification primer 6-5S: | GCTTCAGCTGCTCGATGGGCG |
| | | CAAGTATATTCTGG |
| SEQ ID NO: 46: | pmt6 5′ amplification primer 6-5A: | GGAGACCGGCCTCGAGAAAA |
| | | TCCCGTTCCCTTGGC |
| SEQ ID NO: 47: | pmt6 3′ amplification primer 6-3S: | GAGTCGACCTGCAGGTCTACT |
| | | TGGGATATCGCACC |
| SEQ ID NO: 48: | pmt6 3′ amplification primer 6-3A: | AGCAGCTGAAGCTTGAATAA |
| | | CGCAGCTAAACGAGG |

Then, pSP73-HUH is cleaved with SphI, followed by inserting the 3′ amplified fragment of each gene into the SphI site by use of BD In-fusion Dry-Down PCR Cloning Kit (from Takara Bio Inc.). After transforming E. coli therewith, plasmids are extracted and the nucleotide sequence inserted is identified to provide a plasmid in which the sequence is linked exactly in the intended direction. The resultant plasmid is then cleaved with XhoI, followed by similarly inserting the 5′ amplified fragment of each gene into the XhoI site. After transforming E. coli therewith, plasmids are extracted and the nucleotide sequence inserted is identified to provide a plasmid in which the sequence is linked exactly in the intended direction (each of pSP73-HUH-Dpmt1 to pSP73-HUH-Dpmt6). The resultant pSP73-HUH-Dpmt1 to pSP73-HUH-Dpmt6 are cleaved with PvuII and each used to transform the strain KAY-11. The transformed strain is inoculated into an SD-Ura (2% glucose, 0.67% Yeast Nitrogen Base w/o amino acids (from Difco), nucleobases other than uracil, and an amino acid mixture (20 to 400 mg/L)) medium plate, and cultured at 30° C. for 2 days to provide a transformant.

The resultant transformant is subjected to selection in a YSD medium (1% yeast extract, 2% glucose, adenine (40 mg/L), uracil (20 mg/L)) containing 5-FOA to provide a URA3 gene-deleted strain. The disruption strain in which the URA3 gene is deleted is identified using a PCR method. The resultant strains containing Dpmt1::hisG, Dpmt2::hisG, Dpmt3::hisG, Dpmt4::hisG, Dpmt5::hisG, and Dpmt6::hisG are called strains KAY-13, KAY-14, KAY-15, KAY-16, KAY-17 and KAY-18, respectively.

### Example 10

### Introduction and Expression of the MUC1a Peptide Gene in PMT Gene-Disrupted Mucin-Type Glycopeptide-Producing S. cerevisiae Strains

The MUC1a peptide gene is introduced into the strains KAY-13 to KAY-18 obtained in Example 9 as described in Example 7. Transformants obtained from the strains KAY-13 to KAY-18 are called strains KAY-19 to KAY-24, respectively.

The strains KAY-19 to KAY-24 are each cultured at 30° C. for 48 hours in a YPAD medium (2% polypeptone, 1% yeast extract, 2% glucose, adenine (40 mg/L), followed by centrifugation to provide a culture supernatant. The culture supernatant is adjusted to a pH of 7.0, and TALON metal affinity resins (from Clontech) are then added thereto, which is subsequently washed with a phosphate buffer (pH 7.4) containing 0.5 M NaCl, followed by elution using a phosphate buffer (pH 7.4) containing 0.5 M NaCl and 0.5 M imidazole. The resultant eluate is used as a purified MUC1a preparation.

The purified preparation is subjected to a purity test by HPLC analysis using a reversed-phase column. The column used is Cosmosil 5C18-ARII (4.6×250 mm: from Nacalai Tesque), and the solvents used are 0.05% trifluoroacetic acid (solution A) and 0.05% trifluoroacetic acid-containing acetonitrile (solution B). The column is equilibrated by running the solvent A in advance at a concentration of 0.05% and a flow rate of 0.1 ml/min., and MUC1a is eluted by running the solvent A at a concentration of 0.05% and a flow rate of 0.1 ml/min. for 10 minutes after the injection of the sample and then linearly increasing the proportion of the solvent B to 20% over a period of 20 minutes. The detection is carried out using a UV detector (detection wavelength: 210 nm).

The yeast PMTs have been reported to be active in the form of homo/heterodimers (Girrbach and Strahl, J. Biol. Chem., 278: 12554-12562 (2003)). Thus, it was probable that a PMT double-disrupted or triple-disrupted strain or the like could be properly prepared by a technique similar to that described above to suppress the addition of mannose.

### Example 11

### Expression and Analysis of the MUC1a Peptide Gene in a Mucin-Type Glycopeptide-Producing S. cerevisiae Strain in the Presence of a Compound Inhibiting O-Mannosylation Activity

### A Rhodamine-3-Acetic Acid Derivative

The strain KAY-4 was cultured at 30° C. for 48 hours in a YPAD medium (2% polypeptone, 1% yeast extract, 2% glucose, adenine (40 mg/L) to which rhodamine-3-acetic acid derivative 1c (Orchard et al., Bioorg. Med. Chem. Lett., 14: 3975-3978 (2004)) was added so as to provide a final concentration of 10 μM, followed by centrifugation to give a culture supernatant. The culture supernatant was adjusted to a pH of 7.0, and TALON metal affinity resins (from Clontech) were added thereto, which was then washed with a phosphate buffer (pH 7.4) containing 0.5 M NaCl, followed by elution using a phosphate buffer (pH 7.4) containing 0.5 M NaCl and 0.5 M imidazole. The resultant eluate was used as a purified MUC1a preparation.

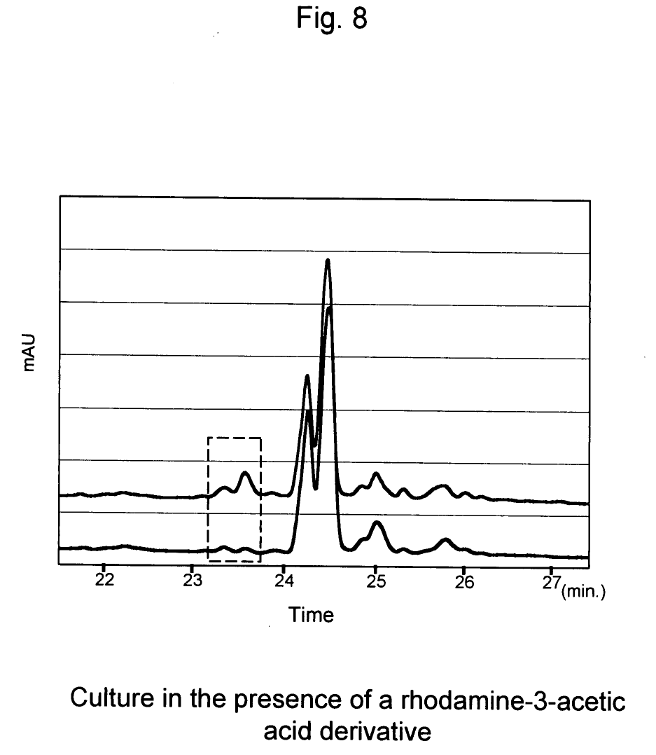

The purified preparation was subjected to a purity test by HPLC analysis using a reversed-phase column. The column used was Cosmosil S5C8-ARII (4.6×250 mm: from Nacalai Tesque), and the solvents used were 0.05% trifluoroacetic acid (solution A) and 0.05% trifluoroacetic acid-containing acetonitrile (solution B). The column was equilibrated by running the solvent A in advance at a concentration of 0.05% and a flow rate of 0.1 ml/min., and MUC1a was eluted by running the solvent A at a concentration of 0.05% and a flow rate of 0.1 ml/min. for 10 minutes after the injection of the sample and then linearly increasing the proportion of the solvent B to 20% over a period of 20 minutes. The detection was carried out using a UV detector (detection wavelength: 210 nm). The results are shown in FIG. 8. A peptide in which mannose as well as GalNAc are added to MUC1a peptide has been found to be eluted at an elution time of 23.5 minutes. A peak at an elution time of 23.5 minutes is found in 6.7% of the total in the absence of the inhibitor, but the percentage is reduced to 1.7% by culture in the presence of the inhibitor. This showed that the addition of rhodamine-3-acetic acid derivative Ic for culture suppressed O-mannosylation unique to yeast in the expression of MUC1a.

### Example 12

### Expression of an FGF Protein in a Mucin-Type Sugar Chain-Adding Strain

Using as a template a plasmid (Yoneda et al., Glycoconj. J. 18, 291-299 (2001)) having a gene encoding a protein in which a pentapeptide glycosylation unit composed of 5 amino acid molecules called ATPAP (hereinafter referred to as a mucin box) is added to the carboxyl terminal of fibroblast growth factor (FGF), an FGF gene containing one mucin box (Cm1) and an FGF gene containing ten mucin box (Cm10) were amplified by PCR employing primer Y (SEQ ID NO: 49) and primer AA (SEQ ID NO: 50). The FGF gene itself for using as a control was also amplified employing primer Y and primer Z (SEQ ID NO: 51).

SEQ ID NO: 49:
GCTCTAGATAAAAGAgctaattacaagaagcccaaac
SEQ ID NO: 50:
GCTCTAGAtggatcggaattcttta
SEQ ID NO: 51:
GCTCTAGAttcttaatcagaagagactgg

These PCR products were cleaved with XbaI, and the FGF gene fragments were incorporated into the XbaI site of pRS303-GAPDH/alfa downstream of the α-factor to prepare plasmids pRS303/GAPDH-alfa-FGF, pRS303/GAPDH-alfa-Cm1 and pRS303/GAPDH-alfa-Cm10. The plasmids were each cleaved with NheI, followed by transforming the strain KAY-3 therewith. The transformation was carried out using a lithium acetate method. The transformed strain was inoculated into an SD-His (2% glucose, 0.17% Yeast Nitrogen Base w/o amino acids (from Difco), nucleobases other than histidine, and an amino acid mixture (20 to 400 mg/L)) medium, and cultured at 30° C. for 2 days to provide a transformant. The transformant was scraped from the plates and confirmed to have been incorporated onto the chromosome by a simplified PCR method involving the suspension thereof in a PCR reaction solution. The strain into which the FGF gene was introduced was called strain KAY-25; the strain into which the Cm1 gene was introduced, strain KAY-26; and the strain into which the Cm10 gene was introduced, strain KAY-27.

A strain used as a control was then prepared by the following procedure. Plasmids pRS303/GAPDH-alfa-FGF, pRS303/GAPDH-alfa-Cm1 and pRS303/GAPDH-alfa-Cm10 were prepared. The plasmids were each cleaved with NheI, followed by transforming the strain KAY-2 therewith. The transformation was carried out using a lithium acetate method. The transformed strain was inoculated into an SD-His (2% glucose, 0.17% Yeast Nitrogen Base w/o amino acids (from Difco), nucleobases other than histidine, and an amino acid mixture (20 to 400 mg/L)) medium, and cultured at 30° C. for 2 days to provide transformants. The transformant was scraped from the plates and confirmed to have been incorporated onto the chromosome by a simplified PCR method involving the suspension thereof in a PCR reaction solution. The strain into which the FGF gene was introduced was called strain KAY-30; the strain into which the Cm1 gene was introduced, strain KAY-28; and the strain into which the Cm10 gene was introduced, strain KAY-29.

The expression and purification of FGF were according to the method of Takamatsu et al. (Takamatsu et al., Glycoconj. J., 20: 385-397 (2004)). The sugar chain was detected by transferring FGF to a PVDF membrane after SDS-PAGE and performing a lectin blotting method. In this respect, the lectin was Jacalin (jackfruit-derived lectin) biotinylated employing a commercial kit (sulfo-NHS-Biotinylation Kit, EZ-link; from Pierce), and a streptavidin-peroxidase complex was reacted for the secondary reaction. The detection was carried out using Immobilon Western peroxidase chemiluminescence Kit from Millipore.

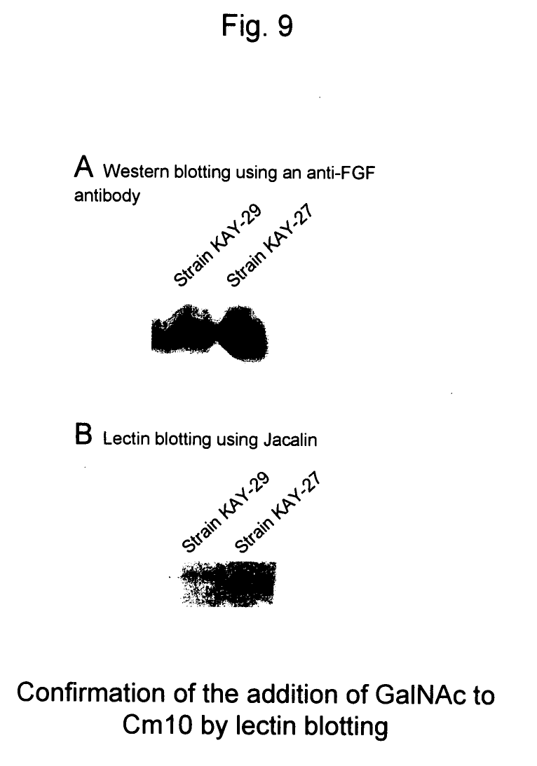

The results are shown in FIG. 9. When the protein amount was determined using a anti-FGF mouse antibody and equal amounts of the samples were analyzed by lectin blotting, the Cm10 derived from strain KAY-27 could be detected with Jacalin, while the Cm 10 derived from strain KAY-29 could not be detected with Jacalin. This may be due to that the addition of GalNAc is made, in the cell, to the Cm10 derived from strain KAY-27, while the addition of GalNAc does not occur, in the cell, to the Cm10 derived from strain KAY-29. These results confirmed that the expression of the protein Cm10 in the strain KAY-27 resulted in the addition of GalNAc to the protein in the cell.

### Example 13

### Production of a S. cerevisiae Strain Producing a Peptide Containing a Mucin-Type Sugar Chain Having a Core 1 Structure

The Drosophila melanogaster core 1 β1-3 GalT (DmGalT) gene is cloned from Drosophila melanogaster mRNA, and the cDNA sequence thereof has been deposited in the public database GenBank under the Accession No. CG9520. First, the catalytic domain of the core 1 β1-3 GalT gene, which had already been cloned, was amplified by PCR using primer W (SEQ ID NO: 52) and primer X (SEQ ID NO: 53).

| | |
|---|---|
| SEQ ID NO: 52: | CCCACTAGTTCCACGCCGGAGCGAAGTG |
| SEQ ID NO: 53: | CGGGGTACCTTATTGCGTCTTTGTCTCGG |

The PCR product was cleaved at the KpnI and SpeI sites and incorporated into the SpeI/KpnI site of plasmid pUC19 (shown above), a plasmid for cloning in E. coli, into which there had already been inserted the gene sequence of the transmembrane domain of MNN9, a subunit of mannosyltransferase in the Golgi apparatus of S. cerevisiae, to construct plasmid pUC19-MNN9/DmGalT. Then, a SpeI site-containing DNA sequence for expressing the FLAG antigen was prepared by PCR using primer K (SEQ ID NO: 21) and primer L (SEQ ID NO: 22) and cleaved at the SpeI site. Subsequently, pUC19-MNN9/DmGalT was cleaved at the SpeI site, and the DNA sequence for expressing the FLAG antigen was inserted into the 5′ region of catalytic domain of DmGalT to prepare pUC19-MNN9/FLAG/DmGalT. A ppGalNAc-T1 gene containing the FLAG antigen gene was cleaved at the BamHI and KpnI sites from the plasmid, blunted at the ends, and inserted into similarly blunted plasmid YEp352GAPII, a plasmid for expression in yeast, at the EcoRI/SalI site to construct YEp352GAPII-MNN9/FLAG/DmGalT. A region containing the yeast GAPDH promoter and terminator was further excised with BamHI from the plasmid, blunted at both ends, and inserted into the ApaI site of similarly blunted pRS304-MNN9/FLAG/ppGalNAc-T1. The plasmid pRS304-MNN9/FLAG/ppGalNAc-T1, MNN9/FLAG/DmGalT was cleaved with EcoRV, followed by transforming the strain KAY-2 therewith. The transformation was carried out using a lithium acetate method. The transformed strain was inoculated into an SD-Trp (2% glucose, 0.17% Yeast Nitrogen Base w/o amino acids (from Difco), nucleobases other than tryptophan, and an amino acid mixture (20 to 400 mg/L)) medium, and cultured at 30° C. for 2 days to provide a transformant. The transformant was scraped from the plate, confirmed to have been incorporated onto the chromosome by a simplified PCR method involving the suspension thereof in a PCR reaction solution, and called strain KAY-31.

### Example 14

### Confirmation of DmGalT Gene Expression in the S. cerevisiae Strain Producing a Peptide Containing a Mucin-Type Sugar Chain Having a Core 1 Structure

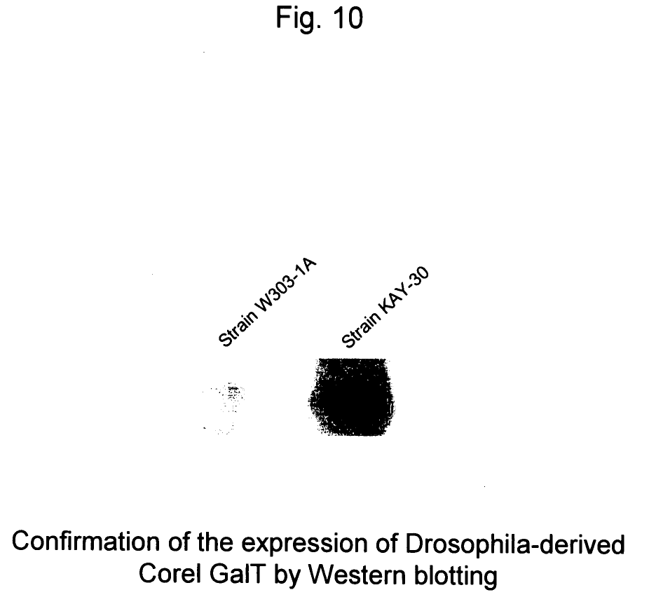

The S. cerevisiae strains KAY-31 and W303-1A were each cultured at 30° C. for 24 hours in 5 ml of a YPAD medium (2% polypeptone, 1% yeast extract, 2% glucose, adenine (40 mg/L)), followed by recovering the cell bodies by centrifugation. The cell bodies were crushed with glass beads, and a surfactant was added to the crush solution to solubilize insoluble protein, followed by centrifugation to provide a supernatant. The resultant supernatant was used as a solution of a crude enzyme, which was then denatured with SDS sample buffer, followed by western blot analysis using an ordinary method. The western blot analysis was performed using the mouse anti-FLAG antibody M2 as a primary antibody and an anti-mouse Ig antibody-horseradish peroxidase complex as a secondary antibody; the detection was carried out on a chemiluminescence detector (Fujifilm Corporation) using a ECL-plus system (from Amersham). The results are shown in FIG. 10.

As shown in FIG. 10, no signal was observed for the strain W303-1A as a control strain, while for the transformant strain the signal was identified at the position of a molecular weight corresponding to a DmGalT gene product with the anti-FLAG antibody.

### Example 15

### Introduction of the MUC1a Gene into the S. cerevisiae Strain Producing a Peptide Containing a Mucin-Type Sugar Chain Having a Core 1 Structure

pRS304-alfaMUC1aHis was prepared and cleaved with NheI, followed by transforming the strain KAY-31 therewith. The transformation was carried out using a lithium acetate method. The transformed strain was inoculated into an SD-His (2% glucose, 0.17% Yeast Nitrogen Base w/o amino acids (from Difco), nucleobases other than histidine, and an amino acid mixture (20 to 400 mg/L)) medium, and cultured at 30° C. for 2 days to provide a transformant. The transformant was scraped from the plate, confirmed to have been incorporated onto the chromosome by a simplified PCR method involving the suspension thereof in a PCR reaction solution, and called strain KAY-32.

### Example 16

### Expression and Analysis of the MUC1a Gene in the S. cerevisiae Strain Producing a Peptide Containing a Mucin-Type Sugar Chain Having a Core 1 Structure

The S. cerevisiae strain KAY-32 was cultured at 30° C. for 72 hours in 100 ml of a YPAD medium (2% polypeptone, 1% yeast extract, 2% glucose, adenine (40 mg/L)), followed by centrifugation to provide a culture supernatant. For a control, the strain KAY-5 was cultured in the same manner as the S. cerevisiae strain KAY-4 to provide a culture supernatant. The culture supernatant was adjusted to a pH of 7.0, and TALON metal affinity resins (from Clontech) were added thereto, which was then washed with a phosphate buffer (pH 7.4) containing 0.5 M NaCl, followed by elution using a phosphate buffer (pH 7.4) containing 0.5 M NaCl and 0.5 M imidazole. The resultant eluate was used as a purified MUC1a preparation.

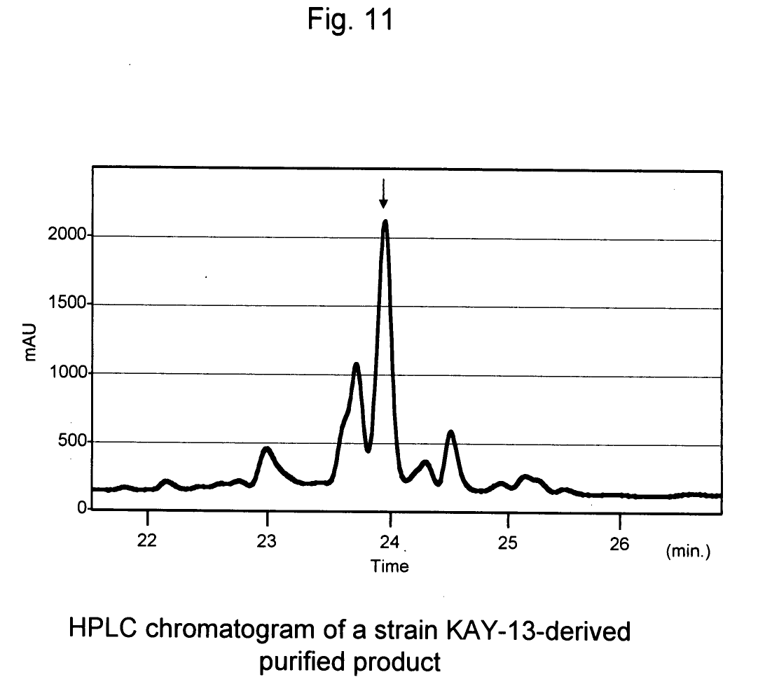

To quantitate the linking amount of GalNAc and Gal for the resultant MUC1a preparation, a purity test was performed by HPLC analysis using a reversed-phase column. The column used was Cosmosil 5C18-ARII (4.6×250 mm: from Nacalai Tesque), and the solvents used were 0.05% trifluoroacetic acid (solution A) and 0.05% trifluoroacetic acid-containing acetonitrile (solution B). The column was equilibrated by running the solvent A in advance at a concentration of 0.05% and a flow rate of 0.1 ml/min., and MUC1a was eluted by running the solvent A at a concentration of 0.05% and a flow rate of 0.1 ml/min. for 10 minutes after the injection of the sample and then linearly increasing the proportion of the solvent B to 20% over a period of 20 minutes. The detection was carried out using a UV detector (detection wavelength: 210 nm). The results are shown in FIG. 11. To test peaks on chromatogram, each peak was recovered.

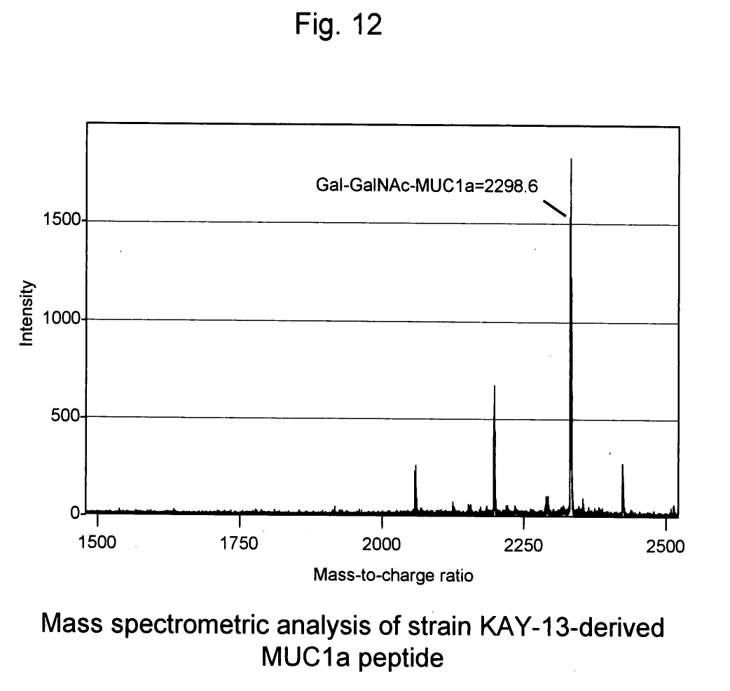

Each peak recovered from HPLC was made into a freeze-dried product and then subjected to molecular weight measurement using MALDI TOF-MS. The results are shown in FIG. 12. Peaks at elution times of about 25.5 min., about 24.5 min., and about 24.0 min. on HPLC were detected at positions of about 1934, about 2137, and 2298, respectively, corresponding to the molecular weights thereof. The molecular weight of the peak at about 25.5 min. agrees with the theoretical value of the MUC1a designed. In contrast, the molecular weight of the peak at about 24.5 min. is in agreement with the molecular weight of MUC1a to which the molecular weight of the GalNAc moiety is added. The peak at about 24.0 min. corresponds to the increased molecular weight of MUC1a due to the addition of GalNAc and Gal thereto. These results demonstrated that the sugar chain Gal-GalNAc was added to MUC1a.

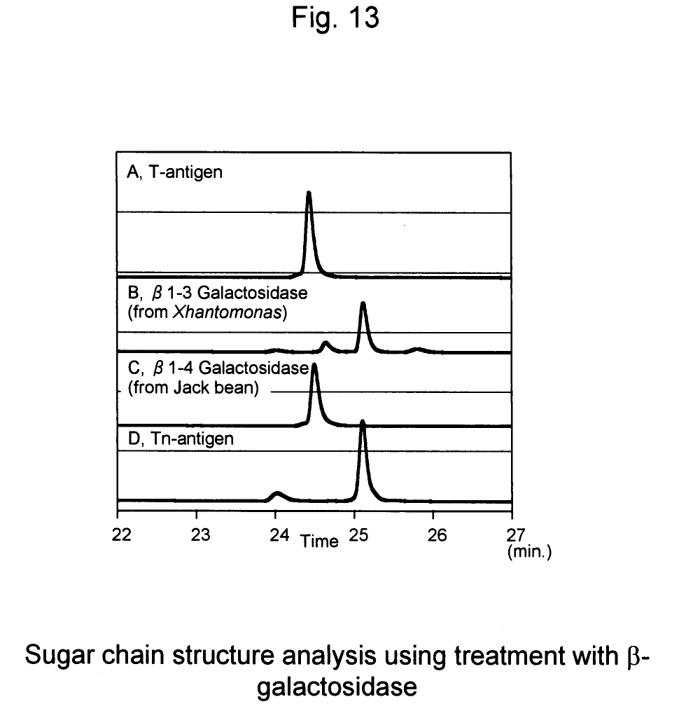

The peak at an elution time of about 24 min. on HPLC was recovered and used as a purified preparation. The purified preparation was treated with a β-galactosidase to analyze the sugar chain structure thereof (FIG. 13). As a result, treatment with β-1,3-galactosidase derived from Xhantomonas (Wong-Madden, S. T. and Landry, D. Glycobiology 5: 19-28 (1995)) produced sugar chain cleavage and caused the treated product to elute at the position of GalNAc-MUC1a, while treatment with β-1,4-galactosidase derived from Jack Bean did not change the elution position thereof.

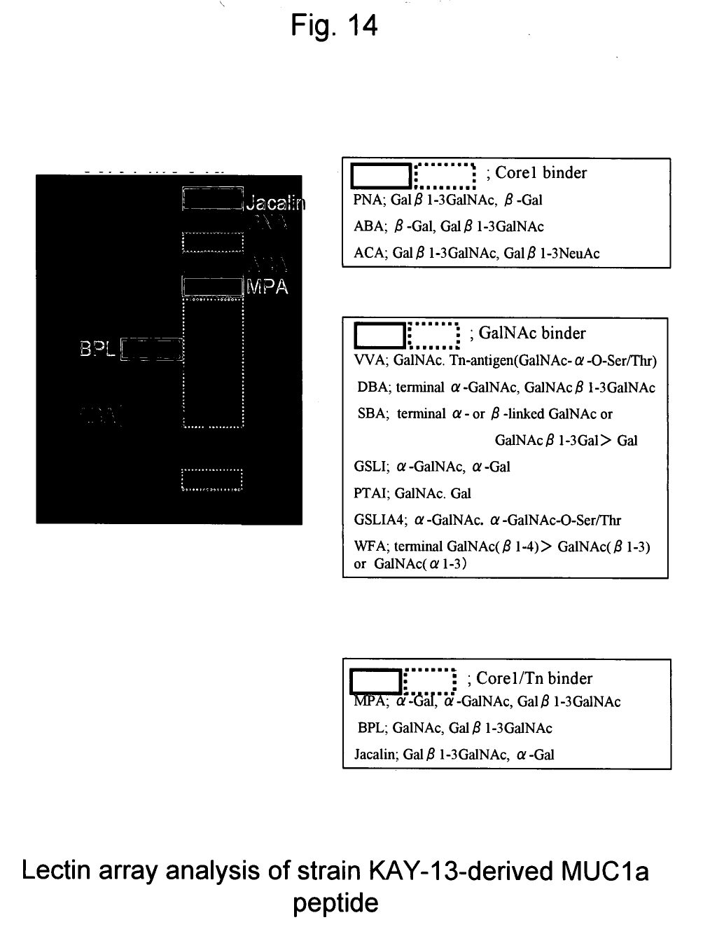

The sugar chain structure of the preparation was also analyzed using a lectin array (Kuno et al., Nat. Methods, 2: 851-856 (2005)). As described in Example 5, the MUC1a was labeled with Cy₃, followed by analyzing the preparation at a concentration of 10 μg/ml using the lectin array. The results are shown in FIG. 14. As a result of the lectin array analysis, the signals were observed on spots of the core 1-specific lectins PNA (peanut-derived lectin), ABA (agaricus-derived lectin), and ACA (amaranthus caudatus-derived lectin). The signals were also observed on spots of the core 1/Tn-specific lectins Jacalin (jackfruit-derived lectin), MPA (osage orange-derived lectin), and BPL (orchid tree-derived lectin). These results showed that the core 1 structure (Galβ1,3GalNAc) was added to the peptide. On the other hand, for the KAY-4-derived peptide prepared as a control, the signals were seen with the Tn-specific lectins, but no signals were observed with the core 1-specific lectins.

### Example 17

### Production of a Mucin-Type Glycopeptide-Producing Strain Using the Methanol-Utilizing Yeast O. minuta

A mucin-type glycopeptide-producing strain was prepared using the methanol-utilizing yeast O. minuta as described below.

First, amplification was performed by PCR technique using pBluescriptII (SK−)-myc/GalE prepared in Example 1 as a template and primer O (SEQ ID NO: 54) and primer P (SEQ ID NO: 55) as primers.

SEQ ID NO: 54:
ACGCGTCGACATGTTAATTAACTCTGAACAAAAACTC
SEQ ID NO: 55:
TCCCGTCGACTTATTCCGCACTCTTATACC

The PCR product was cleaved at the SalI site and inserted into the SalI site of pOMEGPUI to prepare pOMEGPUI-myc/GalE. Similarly, amplification was also carried out by PCR technique using pBluescriptII (SK−)-HA/UGT2 as a template and primer Q (SEQ ID NO: 56) and primer R (SEQ ID NO: 57) as primers.

| | |
|---|---|
| SEQ ID NO: 56: | AAAAAGTCGACAAAATGGCAGCGGTTGGGGCTGG |
| SEQ ID NO: 57: | GCGTGTCGACTTAccaagccgaattaacagcagc |

The PCR product was cleaved at the SalI site and inserted into the SalI site of pOMEGPUI to prepare pOMEGPUI-HA/UGT2. Then, pOMEGPUI-HA/UGT2 was cleaved at the HindIII site and blunted at both ends. Similarly, pOMEGPUI-myc/GalE was cleaved at the HindIII and KpnI sites, and a region comprising O. minuta GAPDH promoter and terminator was further excised, blunted at both ends, and then inserted into the blunted pOMEGPUI-HA/UGT2 at the HindIII site to prepare pOMEGPUI-myc/GalE, HA/UGT2. The plasmid pOMEGPUI-myc/GalE, HA/UGT2 was cleaved with NotI, followed by transforming therewith the O. minuta strain TK5-3 (Δoch1Δura3Δade1) as described in International Publication WO03/091431. The transformation was carried out using a lithium acetate method. The transformed strain was inoculated into an SD-Ura (2% glucose, 0.17% Yeast Nitrogen Base w/o amino acids (from Difco), nucleobases other than uracil, and an amino acid mixture (20 to 400 mg/L)) medium, and cultured at 30° C. for 2 days to provide a transformant. The transformant was scraped from the plate, confirmed to have been incorporated onto the chromosome by a simplified PCR method involving the suspension thereof in a PCR reaction solution, and called strain KAM-1.

To express ppGalNAc-T1 in O. minuta, the amplification thereof was performed by PCR technique using pUC19-MNN9/FLAG/ppGalNAc-T1 as a template and primer S (SEQ ID NO: 58) and primer T (SEQ ID NO: 59) as primers.

| | |
|---|---|
| SEQ ID NO: 58: | ACGATGCATATGTCACTTTCTCTTGTATC |
| SEQ ID NO: 59: | ACGATGCATTCAGAATATTTCTGGCAGGG |

The PCR product was cleaved with EcoT22I and inserted into the EcoT22I of pOMEGPA1 to prepare pOMEGPA1-MNN9/FLAG/ppGalNAc-T1. The plasmid pOMEGPA1-MNN9/FLAG/ppGalNAc-T1 was cleaved with NotI, followed by transforming the strain KAM-1 therewith. The transformation was carried out using a lithium acetate method. The transformed strain was inoculated into an SD-Ade (2% glucose, 0.17% Yeast Nitrogen Base w/o amino acids (from Difco), nucleobases other than adenine, and an amino acid mixture (20 to 400 mg/L)) medium, and cultured at 30° C. for 2 days to provide a transformant. The transformant was scraped from the plate, confirmed to have been incorporated onto the chromosome by a simplified PCR method involving the suspension thereof in a PCR reaction solution, and called strain KAM-2.

### Example 18

### Introduction of the MUC1a Peptide Gene into a Mucin-Type Glycopeptide-Producing Strain Using the Methanol-Utilizing Yeast O. minuta

First, the gene of an MUC1a containing α-factor was amplified by PCR technique using as a template YEp352GAPII-alfaMUC1aHis as prepared in Example I and as primers primer U (SEQ ID NO: 60) and primer V (SEQ ID NO: 61).

| | |
|---|---|
| SEQ ID NO: 60: | ACGCGTCGACATGAGATTTCCTTCAATTTT |
| SEQ ID NO: 61: | CATCACCATCACCATCACTAAGTCGACGCGA |

The PCR product was cleaved at the SalI site and inserted into the SalI site of pOMZeo to prepare pOMZeo-alfaMUC1aHis. The plasmid was cleaved with NotI, followed by transforming the strain KAM-2. The transformed strain was inoculated into a YPAD-Zeocine (2% polypeptone, 1% yeast extract, 2% glucose, adenine (40 mg/L), 100 μg/ml Zeocine) medium and cultured at 30° C. for 2 days to provide a transformant. The transformant was scraped from the plate, confirmed to have been incorporated onto the chromosome by a simplified PCR method involving the suspension thereof in a PCR reaction solution, and called strain KAM-3.

### Example 19

### Production and Analysis of the MUC1a Peptide Gene in the Mucin-Type Glycopeptide-Producing Strain Using the Methanol-Utilizing Yeast O. minuta

The O. minuta strain KAM-3 was cultured at 30° C. for 72 hours in 100 ml of a YPAD medium (2% polypeptone, 1% yeast extract, 2% glucose, adenine (40 mg/L), protease inhibitor cocktail (from Roche)), followed by centrifugation to provide a culture supernatant. The culture supernatant was adjusted to a pH of 7.0, and TALON metal affinity resins (from Clontech) were added thereto, which was then washed with a phosphate buffer (pH 7.4) containing 0.5 M NaCl, followed by elution using a phosphate buffer (pH 7.4) containing 0.5 M NaCl and 0.5 M imidazole. The resultant eluate was used as a purified MUC1a preparation.

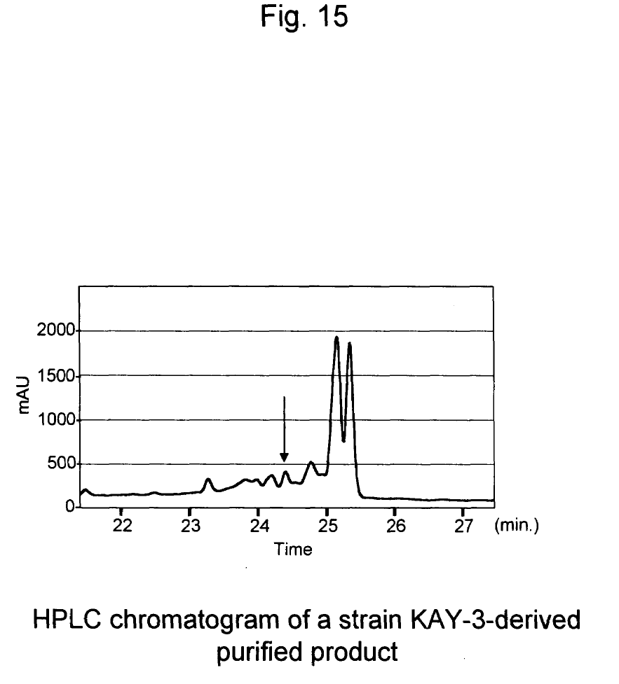

To quantitate the linking amount of GalNAc for the resultant MUC1a preparation, a purity test was performed by HPLC analysis using a reversed-phase column. The column used was Cosmosil C18 (4.6×250 mm: from Nacalai Tesque), and the solvents used were 0.05% trifluoroacetic acid (solution A) and 0.05% trifluoroacetic acid-containing acetonitrile (solution B). The column was equilibrated by running the solvent A in advance at a concentration of 0.05% and a flow rate of 0.1 ml/min., and MUC1a was eluted by running the solvent A at a concentration of 0.05% and a flow rate of 0.1 ml/min. for 10 minutes after the injection of the sample and then linearly increasing the proportion of the solvent B to 20% over a period of 20 minutes. The detection was carried out using a UV detector (detection wavelength: 210 nm). The results are shown in FIG. 15. First, for the strain KAM-3-derived preparation, peaks at positions corresponding to elution times of 24.5 min. and 25.5 min. on HPLC were recovered, and the molecular weight measurement thereof was performed using MALDI TOF-MS.

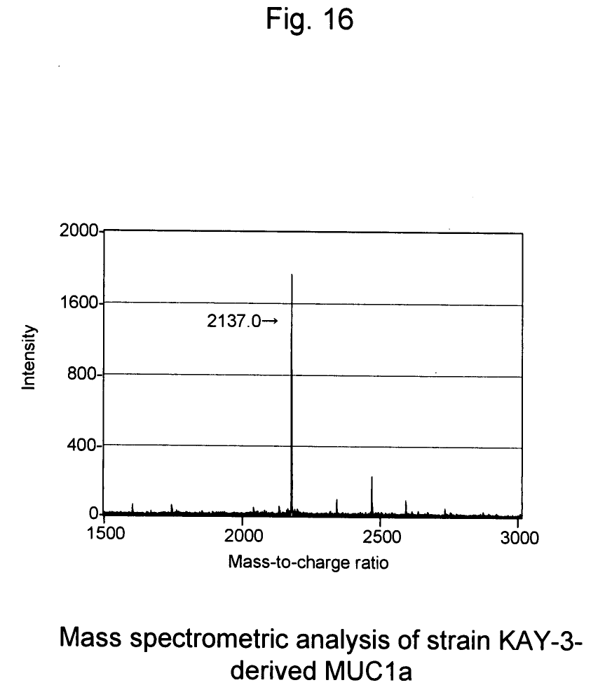

The MUC1a preparations recovered from HPLC were made into freeze-dried products and then subjected to molecular weight measurement using MALDI TOF-MS. The results are shown in FIG. 16. The peak at an elution time of 25.5 min. on HPLC was observed at a position corresponding to a molecular weight of about 1,934, which agreed with the theoretical value of MUC1a. The peak at 24.5 min. was observed at a position corresponding to that of about 2,137, which agreed with a position corresponding to the increased molecular weight of MUC1a due to the addition of GalNAc thereto. These results demonstrated that one GalNAc molecule was linked to MUC1a.

### Example 20

### Production of ppGalNAc-T2-Introduced Mucin-Type Sugar Chain-Producing Yeast Strains

ppGalNAc-T2 gene is located in the human chromosome 1, and the cDNA sequence thereof has been deposited in the public database GenBank under the Accession No. X85019. First, the catalytic domain of the gene was amplified using primer W (SEQ ID NO: 62) and primer X (SEQ ID NO: 63). As a template was used a plasmid (pENTR/D-TOPO-ppGalNAc-T2) into which there was incorporated the ppGalNAc-T2 gene, which had already been cloned in the NEDO “Tosa Idenshi Raiburarino Kochiku (Construction of a glycogene library)” project (Industrial and scientific technology development project for FY 2003: Research Report on Construction of Libraries of Human Genes Participating in Glycosylation Project (Biotechnology Basic Research Program for Health Maintenance and Improvement) subcontracted by New Energy and Industrial Technology Development Organization, from National Institute of Advanced Industrial Science and Technology and Research Association for Biotechnology (2004)) (Shinma and Jigami, Glycoconj. J., Vol. 21: 75-78 (2004)). The PCR product was cleaved at the SpeI and KpnI sites and incorporated into the SpeI/KpnI site of plasmid pUC19, a plasmid for cloning in E. coli, into which there was inserted the gene sequence of the transmembrane domain of MAW9, a subunit of mannosyltransferase in the Golgi apparatus of S. cerevisiae, to prepare plasmid pUC19-MNN9/ppGalNAc-T2.

| | |
|---|---|
| SEQ ID NO: 62: | CCCACTAGTgccggcaggaaggaggactg |
| SEQ ID NO: 63: | CGGGGTACCttactgctgcaggttgagcgtg |

A ppGalNAc-T2 gene containing the MNN9 transmembrane domain gene was amplified using the plasmid as a template and primer Y (SEQ ID NO: 64) and primer Z (SEQ ID NO: 65) as primers, and the EcoRI and SalI sites are further added 5′ and 3′, respectively, thereto. The amplified MNN9-ppGalNAc-T2 fragment was cleaved with EcoRI and SalI and further inserted into the EcoRI and SalI sites of pRS304-MNN9/FLAG/ppGalNAc-T1 cleaved with these two enzymes to prepare pRS304-MNN9/ppGalNAc-T2. The plasmid pRS304-MNN9/ppGalNAc-T2 was cleaved with EcoRV, followed by transforming the strain KAY-2 therewith. The transformation was carried out using a lithium acetate method. The transformed strain was inoculated into an SD-Trp (2% glucose, 0.17% Yeast Nitrogen Base w/o amino acids (from Difco), nucleobases other than tryptophan, and an amino acid mixture (20 to 400 mg/L)) medium, and cultured at 30° C. for 2 days to provide a transformant. The transformant was scraped from the plate, confirmed to have been incorporated onto the chromosome by a simplified PCR method involving the suspension thereof in a PCR reaction solution, and called strain KAY-33.

| | |
|---|---|
| SEQ ID NO: 64: | ccggaattcatgtcactttctcttgtatc |
| SEQ ID NO: 65: | acgcgtcgacttactgctgcaggttgagcg |

In addition, blunted at both ends, a MNN9/FLAG/DmGalT gene fragment containing the GAPDH promoter and terminator sequences used in Example 13 was inserted into a plasmid obtained by cleaving pRS304-MNN9/ppGalNAc-T2 at the ApaI site and blunting at both ends. The resultant plasmid was called pRS304-MNN9/ppGlaNAc-T2, MNN9/FLAG/DmGalT. The plasmid pRS304-MNN9/ppGlaNAc-T2, MNN9/FLAG/DmGalT was cleaved with EcoRV, followed by transforming the strain KAY-2 therewith. The transformation was carried out using a lithium acetate method. The transformed strain was inoculated into an SD-Trp (2% glucose, 0.17% Yeast Nitrogen Base w/o amino acids (from Difco), nucleobases other than tryptophan, and an amino acid mixture (20 to 400 mg/L)) medium, and cultured at 30° C. for 2 days to provide a transformant. The transformant was scraped from the plate, confirmed to have been incorporated onto the chromosome by a simplified PCR method involving the suspension thereof in a PCR reaction solution, and called strain KAY-34.

### Example 21

### Production of ppGalNAc-T3-Introduced Mucin-Type Sugar Chain-Producing Yeast Strains

ppGalNAc-T3 gene is located in the human chromosome 2, and the cDNA sequence thereof has been deposited in the public database GenBank under the Accession No. X92689. First, the catalytic domain of the gene was amplified using primer AA (SEQ ID NO: 66) and primer AB (SEQ ID NO: 67). As a template was used a plasmid (pENTR/D-TOPO-ppGalNAc-T3) into which there was incorporated the ppGalNAc-T3 gene, which had already been cloned in the NEDO “Tosa Idenshi Raiburarino Kochiku (Construction of a glycogene library)” project (Industrial and scientific technology development project for FY 2003: Research Report on Construction of Libraries of Human Genes Participating in Glycosylation Project (Biotechnology Basic Research Program for Health Maintenance and Improvement) subcontracted by New Energy and Industrial Technology Development Organization, from National Institute of Advanced Industrial Science and Technology and Research Association for Biotechnology (2004)). The PCR product was cleaved at the SpeI and KpnI sites and incorporated into the SpeI/KpnI site of plasmid pUC19, a plasmid for cloning in E. coli, into which there was inserted the gene sequence of the transmembrane domain of MNN9, a subunit of mannosyltransferase in the Golgi apparatus of S. cerevisiae, to prepare plasmid pUC19-MNN9/ppGalNAc-T3.

| | |
|---|---|
| SEQ ID NO: 66: | CCCACTAGTcaaagagaagtaagtgttc |
| SEQ ID NO: 67: | CGGGGTACCttaatcattttggctaagtatc |

A ppGalNAc-T3 gene containing the MNN9 transmembrane domain gene was amplified using the plasmid as a template and primer AC (SEQ ID NO: 68) and primer AD (SEQ ID NO: 69) as primers, and the MfeI and SalI sites are further added 5′ and 3′, respectively, thereto. The amplified MNN9-ppGalNAc-T3 fragment was cleaved with MfeI and SalI and further inserted into the EcoRI and SalI sites of pRS304-MNN9/FLAG/ppGalNAc-T1 cleaved with these two enzymes to prepare pRS304-MNN9/ppGalNAc-T3. The plasmid pRS304-MNN9/ppGalNAc-T3 was cleaved with EcoRV, followed by transforming the strain KAY-2 therewith. The transformation was carried out using a lithium acetate method. The transformed strain was inoculated into an SD-Trp (2% glucose, 0.17% Yeast Nitrogen Base w/o amino acids (from Difco), nucleobases other than tryptophan, and an amino acid mixture (20 to 400 mg/L)) medium, and cultured at 30° C. for 2 days to provide a transformant. The transformant was scraped from the plate, confirmed to have been incorporated onto the chromosome by a simplified PCR method involving the suspension thereof in a PCR reaction solution, and called strain KAY-35.

| | |
|---|---|
| SEQ ID NO: 68: | ccgcaattgatgtcactttctcttgtatc |
| SEQ ID NO: 69: | acgcgtcgacttaatcattttggctaagtatc |

In addition, blunted at both ends, a MNN9/FLAG/DmGalT gene fragment containing the GAPDH promoter and terminator sequences used in Example 13 was inserted into a plasmid obtained by cleaving pRS304-MNN9/ppGalNAc-T3 at the SmaI site. The resultant plasmid was called pRS304-MNN9/ppGlaNAc-T3, MNN9/FLAG/DmGalT. The plasmid pRS304-MNN9/ppGlaNAc-T3, MNN9/FLAG/DmGalT was cleaved with EcoRV, followed by transforming the strain KAY-2 therewith. The transformation was carried out using a lithium acetate method. The transformed strain was inoculated into an SD-Trp (2% glucose, 0.17% Yeast Nitrogen Base w/o amino acids (from Difco), nucleobases other than tryptophan, and an amino acid mixture (20 to 400 mg/L)) medium, and cultured at 30° C. for 2 days to provide a transformant. The transformant was scraped from the plate, confirmed to have been incorporated onto the chromosome by a simplified PCR method involving the suspension thereof in a PCR reaction solution, and called strain KAY-36.

### Example 22

### Production of a Core 2 Structure-Producing Yeast Strain

The MNN2-2 gene derived from Kluyveromyces lactis is located on the Kluyveromyces lactis genome, and the cDNA sequence thereof has been deposited in the public database GenBank under the Accession No. U48413. The full-length cDNA (SEQ ID NO: 84) of the MNN2-2 gene was amplified by PCR technique using the genome of Kluyveromyces lactis strain as a template and primer AG (SEQ ID NO: 70) and primer AH (SEQ ID NO: 71).

| | |
|---|---|
| SEQ ID NO: 70: | CTAGAGCTCATGAGTTTTGTATTGATTTTGTC |
| SEQ ID NO: 71: | TCGCGTCGACTCAGCGAGGCAGTGCAGTTTTGAC |

The resultant PCR product was cleaved at the SacI and SalI sites and inserted into the SacI and SalI sites of plasmid YEp352GAPII, a plasmid for expression in yeast, to provide YEp352GAPII-MNN2-2. In addition, a region containing the yeast GAPDH promoter and terminator was excised with BamHI from the plasmid and blunted at both ends. Further, the pRS305-HA/IUGT2 prepared in Example 1 was cleaved at the SacI site and blunted at both ends to prepare pRS305-HA/UGT2/MNN2-2. The plasmid pRS305-HA/UGT2/MNN2-2 was cleaved with EcoRV, followed by transforming the S. cerevisiae strain W303-1A therewith. The transformation was carried out using a lithium acetate method. The transformed strain was inoculated into an SD-Leu (2% glucose, 0.17% Yeast Nitrogen Base w/o amino acids (from Difco), nucleobases other than leucine, and an amino acid mixture (20 to 400 mg/L)) medium, and cultured at 30° C. for 2 days to provide a transformant. The transformant was scraped from the plate, confirmed to have been incorporated onto the chromosome by a simplified PCR method involving the suspension thereof in a PCR reaction solution, and called strain KAY-37.

The pRS304-MNN9/ppGalNAc-T1,MNN9/FLAG/DmGalT prepared in Example 13 was cleaved with EcoRV, followed by transforming the strain KAY-37 therewith. The transformation was carried out using a lithium acetate method. The transformed strain was inoculated into an SD-Trp (2% glucose, 0.17% Yeast Nitrogen Base w/o amino acids (from Difco), nucleobases other than tryptophan, and an amino acid mixture (20 to 400 mg/L)) medium, and cultured at 30° C. for 2 days to provide a transformant. The transformant was scraped from the plate, confirmed to have been incorporated onto the chromosome by a simplified PCR method involving the suspension thereof in a PCR reaction solution, and called strain KAY-38.

The core 2 GnT-1 (C2GnT1) gene is a core 2 structure-synthesizing enzyme gene, and the cDNA sequence thereof has been deposited in the public database GenBank under the Accession No. BC074885. Using as a template a plasmid (pENTR/D-TOPO-C2GnT1) into which there was incorporated the C2GnT1 gene, which had already been cloned in the NEDO “Tosa Idenshi Raiburarino Kochiku (Construction of a glycogene library)” project (Industrial and scientific technology development project for FY 2003: Research Report on Construction of Libraries of Human Genes Participating in Glycosylation Project (Biotechnology Basic Research Program for Health Maintenance and Improvement) subcontracted by New Energy and Industrial Technology Development Organization, from National Institute of Advanced Industrial Science and Technology and Research Association for Biotechnology (2004)), the catalytic domain of the C2GnT1 gene was amplified by PCR technique employing primer AI (SEQ ID NO: 72) and primer AJ (SEQ ID NO: 73).

| | |
|---|---|
| SEQ ID NO: 72: | CCCACTAGTagacacttggagcttgctg |
| SEQ ID NO: 73: | CGGGGTACCtcagtgttttaatgtctc |

The PCR product was cleaved at the SpeI and KpnI sites and incorporated into the SpeI/KpnI site of plasmid pUC19, a plasmid for cloning in E. coli, into which there is inserted the gene sequence of the transmembrane domain of MNN9, a subunit of mannosyltransferase in the Golgi apparatus of S. cerevisiae, to prepare plasmid pUC19-MNN9/C2GnT1. Then, a SpeI site-containing DNA sequence for expressing the FLAG antigen was prepared by PCR using primer K (SEQ ID NO: 21) and primer L (SEQ ID NO: 22) and cleaved at the SpeI site. Subsequently, the pUC19-MNN9/C2GnT1 was cleaved at the SpeI site, and the DNA sequence for expressing the FLAG antigen was inserted into the 5′ region of catalytic domain of C2GnT1 to prepare pUC19-MNN9/FLAG/C2GnT1. The C2GnT1 gene containing the FLAG antigen gene was cleaved at the BamHI and KpnI sites, from the plasmid, blunted at the ends, and inserted into similarly blunted plasmid YEp352GAPII, a plasmid for expression in yeast, at the EcoRI/SalI site to construct YEp352GAPII-MNN9/FLAG/C2GnT1. A region containing the yeast GAPDH promoter and terminator was further excised with BamHI from YEp352GAPII-MNN9/FLAG/C2GnT1, blunted at both ends, and inserted into a plasmid obtained by cleaving the pRS306-myc/GalE prepared in Example 1 at the SacI site and blunting at both ends to prepare pRS306-myc/GalE, MNN9/FLAG/C2GnT1. The plasmid pRS306-myc/GalE, MNN9/FLAG/C2GnT1 was cleaved with EcoRV, followed by transforming the strain KAY-38 therewith. The transformation was carried out using a lithium acetate method. The transformed strain was inoculated into an SD-Ura (2% glucose, 0.17% Yeast Nitrogen Base w/o amino acids (from Difco), nucleobases other than uracil, and an amino acid mixture (20 to 400 mg/L)) medium, and cultured at 30° C. for 2 days to provide a transformant. The transformant was scraped from the plate, confirmed to have been incorporated onto the chromosome by a simplified PCR method involving the suspension thereof in a PCR reaction solution, and called strain KAY-39.

The plasmid pRS303-alfaMUC1aHis prepared in Example 2 was cleaved with NheI, followed by transforming the strain KAY-39 therewith. The transformation was carried out using a lithium acetate method. The transformed strain was inoculated into an SD-His (2% glucose, 0.17% Yeast Nitrogen Base w/o amino acids (from Difco), nucleobases other than histidine, and an amino acid mixture (20 to 400 mg/L)) medium, and cultured at 30° C. for 2 days to provide a transformant. The transformant was scraped from the plate, confirmed to have been incorporated onto the chromosome by a simplified PCR method involving the suspension thereof in a PCR reaction solution, and called strain KAY-40.

### Example 23

### Production of a Core 3 Structure-Producing Yeast Strain

β3GnT6 is a core 3 structure-synthesizing enzyme gene, and the cDNA sequence thereof has been deposited in the public database GenBank under the Accession No. AB073740. Using as a template a plasmid (pENTR/D-TOPO-β3GnT6) into which there was incorporated the β3GnT6 gene, which had already been cloned in the NEDO “Tosa Idenshi Raiburarino Kochiku (Construction of a glycogene library)” project (Industrial and scientific technology development project for FY 2003: Research Report on Construction of Libraries of Human Genes Participating in Glycosylation Project (Biotechnology Basic Research Program for Health Maintenance and Improvement) subcontracted by New Energy and Industrial Technology Development Organization, from National Institute of Advanced Industrial Science and Technology and Research Association for Biotechnology (2004)), the catalytic domain of the β3GnT6 gene was amplified by PCR technique employing primer AK (SEQ ID NO: 74) and primer AL (SEQ ID NO: 75).

| | |
|---|---|
| SEQ ID NO: 74: | GACTAGTcaggaggagacgccagaggg |
| SEQ ID NO: 75: | GGAATTCttaggagacccggtgtccccgg |

The PCR product was cleaved at the SpeI and EcoRI sites and incorporated into the SpeI/EcoRI site of plasmid pUC19, a plasmid for cloning in E. coli, into which there is inserted the gene sequence of the transmembrane domain of MNN9, a subunit of mannosyltransferase in the Golgi apparatus of S. cerevisiae, to prepare plasmid pUC19-MNN9/β3GnT6. The β3GnT6 gene was cleaved at the BamHI and EcoRI sites from the plasmid, blunted at the ends, and then inserted into similarly blunted plasmid YEp352GAPII, a plasmid for expression in yeast, at the EcoRI/SalI site to construct YEp352GAPII-MNN9/β3GnT6. In addition, a region containing the yeast GAPDH promoter and terminator was excised with BamHI from the plasmid, blunted at both ends, and inserted into a plasmid obtained by cleaving pRS304-MNN9/FLAG/ppGalNAc-T1 prepared in Example 1 at the ApaI site and blunting at both ends. The resultant plasmid pRS304-MNN9/FLAG/ppGalNAc-T1, MNN9/β3GnT6 was cleaved with EcoRV, followed by transforming the strain KAY-37 therewith. The transformation was carried out using a lithium acetate method. The transformed strain was inoculated into an SD-Trp (2% glucose, 0.17% Yeast Nitrogen Base w/o amino acids (from Difco), nucleobases other than tryptophan, and an amino acid mixture (20 to 400 mg/L)) medium, and cultured at 30° C. for 2 days to provide a transformant. The transformant was scraped from the plate, confirmed to have been incorporated onto the chromosome by a simplified PCR method involving the suspension thereof in a PCR reaction solution, and called strain KAY-41.

The pRS306-myc/GalE prepared in Example 1 was cleaved with EcoRV, followed by transforming the strain KAY-41 therewith. The transformation was carried out using a lithium acetate method. The transformed strain was inoculated into an SD-Ura (2% glucose, 0.17% Yeast Nitrogen Base w/o amino acids (from Difco), nucleobases other than uracil, and an amino acid mixture (20 to 400 mg/L)) medium, and cultured at 30° C. for 2 days to provide a transformant. The transformant was scraped from the plate, confirmed to have been incorporated onto the chromosome by a simplified PCR method involving the suspension thereof in a PCR reaction solution, and called strain KAY-42.

The plasmid pRS303-alfaMUC1aHis prepared in Example 2 was cleaved with NheI, followed by transforming the strain KAY-42 therewith. The transformation was carried out using a lithium acetate method. The transformed strain was inoculated into an SD-His (2% glucose, 0.17% Yeast Nitrogen Base w/o amino acids (from Difco), nucleobases other than histidine, and an amino acid mixture (20 to 400 mg/L)) medium, and cultured at 30° C. for 2 days to provide a transformant. The transformant was scraped from the plate, confirmed to have been incorporated onto the chromosome by a simplified PCR method involving the suspension thereof in a PCR reaction solution, and called strain KAY-43.

All publications, patents and patent applications cited herein are incorporated herein by reference in their entirety.

### INDUSTRIAL APPLICABILITY

According to the present invention, a glycoprotein having a mucin-type sugar chain can be inexpensively produced on a large scale. The present invention can be used for the development of various therapies such as a cancer immunotherapy using a mucin-type glycopeptide as an antigen, and for the drug development including the preparation of an antibody to a mucin-type glycoprotein-specific antigen present on the surface of cancer cells and the application of the antibody to an antibody drug.

### FREE TEXT OF SEQUENCE LISTING

SEQ ID NO: 5: Description of Artificial Sequence: Gene encoding a protein in which the transmembrane region of Mnn9 protein is fused to the catalytic region of ppGalNAc T1
SEQ ID NO: 6: Description of Artificial Sequence: Protein in which the transmembrane region of Mnn9 protein is fused to the catalytic region of ppGalNAc T1
SEQ ID NO: 9: Description of Artificial Sequence: Gene encoding a protein in which the transmembrane region of Mnn9 protein is fused to the catalytic region of Drosophila β-1,3-Gal transferase
SEQ ID NO: 10: Description of Artificial Sequence: Protein in which the transmembrane region of Mnn9 protein is fused to the catalytic region of Drosophila β-1,3-Gal transferase

### SEQ ID NOS: 11 to 75: Description of Artificial Sequence: Primers

SEQ ID NO: 76: Description of Artificial Sequence: Gene encoding a protein in which the transmembrane region of Mnn9 protein is fused to the catalytic region of ppGalNAc T2
SEQ ID NO: 77: Description of Artificial Sequence: Protein in which the transmembrane region of Mnn9 protein is fused to the catalytic region of ppGalNAc T2
SEQ ID NO: 78: Description of Artificial Sequence: Gene encoding a protein in which the transmembrane region of Mnn9 protein is fused to the catalytic region of ppGalNAc T3
SEQ ID NO: 79: Description of Artificial Sequence: Protein in which the transmembrane region of Mnn9 protein is fused to the catalytic region of ppGalNAc T3
SEQ ID NO: 80: Description of Artificial Sequence: Gene encoding a protein in which the transmembrane region of Mnn9 protein is fused to FLAG antigen and the catalytic region of C2GnT1
SEQ ID NO: 81: Description of Artificial Sequence: Protein in which the transmembrane region of Mnn9 protein is fused to FLAG antigen and the catalytic region of C2GnT1
SEQ ID NO: 82: Description of Artificial Sequence: Gene encoding a protein in which the transmembrane region of Mnn9 protein is fused to β3GnT6
SEQ ID NO: 83: Description of Artificial Sequence: Protein in which the transmembrane region of Mnn9 protein is fused to β3GnT6
SEQ ID NO: 84: Full-length cDNA of MNN2-2 gene
SEQ ID NO: 85: Mnn2-2

What is claimed is:

1. A method for producing a transformed yeast for generating a mucin-type glycoprotein having O-GalNAc, comprising the step of introducing into a yeast at least one selected from the group consisting of a gene encoding UDP-GalNAc synthetase, a gene encoding UDP-GalNAc transporter, and a gene encoding polypeptide:O-GalNAc transferase.

2. The method according to claim 1, wherein the method comprises the steps of:
(1) introducing the gene encoding UDP-GalNAc synthetase; and
(2) introducing the gene encoding UDP-GalNAc transporter into the yeast.

3. The method according to claim 1, wherein the method comprises the steps of:
(1) introducing the gene encoding UDP-GalNAc synthetase; and
(2) introducing the gene encoding polypeptide:O-GalNAc transferase into the yeast.

4. The method according to claim 1, wherein the method comprises the steps of:
(1) introducing the gene encoding UDP-GalNAc transporter; and
(2) introducing the gene encoding polypeptide:O-GalNAc transferase into the yeast.

5. The method according to claim 1, wherein the method comprises the steps of:
(1) introducing the gene encoding UDP-GalNAc synthetase;
(2) introducing the gene encoding UDP-GalNAc transporter; and
(3) introducing the gene encoding polypeptide:O-GalNAc transferase into the yeast.

6. The method according to claim 1, wherein the polypeptide:O-GalNAc transferase comprises the transmembrane domain of a protein localized in the yeast Golgi apparatus.

7. A method for producing a transformed yeast for generating a mucin-type glycoprotein having O-GalNAc, comprising the step of further introducing a gene encoding a mucin-type glycoprotein into the yeast obtained by the method according to claim 1.

8. The method according to claim 7, wherein the gene encoding a mucin-type glycoprotein comprises a secretion signal sequence derived from yeast.

9. A method for producing a transformed yeast for generating a mucin-type glycoprotein having O-GalNAc, comprising the step of further introducing a gene encoding core 1 β1-3 Gal transferase, core 2 β-1,6-GlcNAc transferase, or core 3 β-1,3-GlcNAc transferase into the yeast obtained by the method according to claim 1.

10. The method according to claim 9, wherein the core 1 β1-3 Gal transferase comprises the transmembrane domain of a protein localized in the yeast Golgi apparatus.

11. A method for producing a transformed yeast for generating a mucin-type glycoprotein having O-GalNAc, comprising further performing the steps of:
(1) introducing a gene encoding UDP-Gal synthetase into the yeast obtained by the production method according to claim 9, when the UDP-GalNAc synthetase has no UDP-Gal synthetic activity; and/or
(2) introducing a gene encoding a UDP-Gal transporter into the yeast obtained by the production method according to claim 9, when the UDP-GalNAc transporter has no UDP-Gal transport activity.

12. A method for producing a transformed yeast for generating a mucin-type glycoprotein having O-GalNAc, comprising the step of further introducing a gene encoding a mucin-type glycoprotein into the yeast obtained by the method according to claim 9.

13. The method according to claim 12, wherein the gene encoding a mucin-type glycoprotein comprises a secretion signal sequence derived from yeast.

14. The method according to claim 1, wherein the yeast is a methanol-utilizing yeast.

15. The method according to claim 14, wherein the methanol-utilizing yeast is Ogataea minuta.

16. The method according to claim 1, wherein the yeast is Saccharomyces cerevisiae.

17. A transformed yeast obtained by the method according to claim 1.

18. A method for producing a mucin-type glycoprotein, comprising the steps of:
culturing the transformed yeast according to claim 17; and
collecting the mucin-type glycoprotein from the culture.

19. The method according to claim 18, further comprising the step of exposure to a compound inhibiting the activity of an O-mannosylation enzyme.

20. A mucin-type glycoprotein obtained by the method according to claim 18.

21. A method for producing a transformed yeast for generating a mucin-type glycoprotein having O-GalNAc, comprising the step of further disrupting an enzyme having O-mannosilation activity in the yeast obtained by the method according to claim 1.

22. A method for producing a transformed yeast for generating a mucin-type glycoprotein having O-GalNAc, comprising the step of further introducing a gene encoding a mucin-type glycoprotein into the yeast obtained by the method according to claim 21.

23. The method according to claim 22, wherein the gene encoding a mucin-type glycoprotein comprises a secretion signal sequence derived from yeast.

24. A transformed yeast obtained by the method according to claim 21.

25. A method for producing a mucin-type glycoprotein, comprising the steps of:
culturing the transformed yeast according to claim 24; and
collecting the mucin-type glycoprotein from the culture.

26. A mucin-type glycoprotein obtained by the method according to claim 25.
